(19) United States
(12) Patent Application Publication
(10) Pub. No.: US 2026/0347467 A1
(43) Pub. Date: Nov. 5, 2026

(54) ANTI-TREM2 ANTIBODIES AND METHODS OF USE THEREOF

(71) Applicant: Denali Therapeutics Inc., South San Francisco, CA (US)

(72) Inventors: Mark S. Dennis, South San Francisco, CA (US); Sherie Duncan, South San Francisco, CA (US); Kathleen Lisaingo, South San Francisco, CA (US); Kathryn M. Monroe, South San Francisco, CA (US); Joshua I. Park, South San Francisco, CA (US); Rachel Prorok, South San Francisco, CA (US); Ju Shi, South San Francisco, CA (US); Ankita Srivastava, South San Francisco, CA (US); Bettina Van Lengerich, South San Francisco, CA (US); Riley Walsh, South San Francisco, CA (US)

(73) Assignee: Denali Therapeutics Inc., South San Francisco, CA (US)

(21) Appl. No.: 19/734,866

(22) Filed: Jul. 8, 2026

Related U.S. Application Data
(63) Continuation of application No. 19/340,626, filed on Sep. 25, 2025.
Application No. 19/340,626 is a continuation of application No. 17/403,406, filed on Aug. 16, 2021, now abandoned.
Application No. 17/403,406 is a continuation of application No. PCT/US2020/019093, filed on Feb. 20, 2020.
(60) Provisional application No. 62/808,141, filed on Feb. 20, 2019.

(51) Int. Cl. C07K 16/28; A61K 39/00

(52) U.S. Cl. CPC C07K 16/2803; A61K 2039/505; C07K 2317/24; C07K 2317/565; C07K 2317/75; C07K 2317/92

(57) ABSTRACT

In one aspect, antibodies that specifically bind to a human triggering receptor expressed on myeloid cells 2 (TREM2) protein are provided. In some embodiments, the antibody decreases levels of soluble TREM2 (sTREM2). In some embodiments, the antibody enhances TREM2 activity.

### CROSS-REFERENCE TO RELATED APPLICATIONS

This application is a continuation of U.S. application Ser. No. 19/340,626, filed Sep. 25, 2025, which is continuation of U.S. application Ser. No. 17/403,406, filed Aug. 16, 2021, now abandoned, which is a continuation of International Application No. PCT/US2020/019093, filed Feb. 20, 2020, which claims priority to U.S. Provisional Application No. 62/808,141, filed Feb. 20, 2019, the disclosures of which are hereby incorporated by reference in their entirety for all purposes.

### SEQUENCE LISTING

The instant application contains a Sequence Listing which has been submitted electronically in XML format and is hereby incorporated by reference in its entirety. Said sequence listing, created on Sep. 25, 2025, is named 639369SEQLIST.xml and is 115,884 bytes in size.

### BACKGROUND

Triggering receptor expressed on myeloid cells-2 (TREM2) is a transmembrane receptor that is expressed on microglia and is believed to function in regulating phagocytosis, cell survival, and the production of pro-inflammatory cytokines. Mutations in TREM2 have been identified in neurodegenerative diseases including Alzheimer's disease, Nasu-Hakola disease, Parkinson's disease, amyotrophic lateral sclerosis, and frontotemporal dementia. Additionally, altered levels of soluble TREM2 (sTREM2) have been reported in the cerebrospinal fluid of patients having Alzheimer's disease or frontotemporal dementia who have a mutation in TREM2.

There remains a need for therapeutic agents that modulate TREM2 activity or levels of sTREM2.

### BRIEF SUMMARY

In one aspect, isolated antibodies or antigen-binding fragments thereof that specifically binds to a human triggering receptor expressed on myeloid cells 2 (TREM2) are provided. In some embodiments, the antibody or antigen-binding fragment thereof that specifically binds to TREM2 comprises:
(a) a CDR-H1 sequence comprising the sequence of GFSIEDFYIH (SEQ ID NO:29);
(b) a CDR-H2 sequence comprising the sequence of W-I-D-P-E-X₆-G-X₈-S-K-Y-A-P-K-F-Q-G (SEQ ID NO:47), wherein X₆ is N or Q and X₈ is D or E;
(c) a CDR-H3 sequence comprising the sequence of HADHGNYGSTMDY (SEQ ID NO:31);
(d) a CDR-L1 sequence comprising the sequence of HASQHINVWLS (SEQ ID NO:32);
(e) a CDR-L2 sequence comprising the sequence of KASNLHT (SEQ ID NO:33); and
(f) a CDR-L3 sequence comprising the sequence of QQGQTYPRT (SEQ ID NO:34).

In some embodiments, the CDR-H2 sequence is selected from SEQ ID NOS:30, 39, 41, and 43.

In some embodiments, the antibody or antigen-binding fragment comprises:
(a) a CDR-H1 comprising the amino acid sequence of SEQ ID NO:29, a CDR-H2 comprising the amino acid sequence of SEQ ID NO:30, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(b) a CDR-H1 comprising the amino acid sequence of SEQ ID NO:29, a CDR-H2 comprising the amino acid sequence of SEQ ID NO:39, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(c) a CDR-H1 comprising the amino acid sequence of SEQ ID NO:29, a CDR-H2 comprising the amino acid sequence of SEQ ID NO:41, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(d) a CDR-H1 comprising the amino acid sequence of SEQ ID NO:29, a CDR-H2 comprising the amino acid sequence of SEQ ID NO:43, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34.

In some embodiments, the antibody or antigen-binding fragment comprises a V_{H} sequence that has at least 85% sequence identity to any one of SEQ ID NOS:27, 35, 37, 38, 40, 42, 44, 45, and 46. In some embodiments, the V_{H} sequence has at least 90% sequence identity to SEQ ID NO:27. In some embodiments, the V_{H} sequence has at least 95% sequence identity to SEQ ID NO:27. In some embodiments, the V_{H} sequence comprises SEQ ID NO:27. In some embodiments, the V_{H} sequence has at least 90% sequence identity to SEQ ID NO:42. In some embodiments, the V_{H} sequence has at least 95% sequence identity to SEQ ID NO:42. In some embodiments, the V_{H} sequence comprises SEQ ID NO:42. In some embodiments, the V_{H} sequence has at least 90% sequence identity to SEQ ID NO:45. In some embodiments, the V_{H} sequence has at least 95% sequence identity to SEQ ID NO:45. In some embodiments, the V_{H} sequence comprises SEQ ID NO:45.

In some embodiments, the antibody or antigen-binding fragment comprises a V_{L} sequence that has at least 85% sequence identity to SEQ ID NO:28 or SEQ ID NO:36. In some embodiments, the V_{L} sequence has at least 90% sequence identity to SEQ ID NO:28. In some embodiments, the V_{L} sequence has at least 95% sequence identity to SEQ ID NO:28. In some embodiments, the V_{L} sequence comprises SEQ ID NO:28. In some embodiments, the V_{L} sequence has at least 90% sequence identity to SEQ ID NO:36. In some embodiments, the V_{L} sequence has at least 95% sequence identity to SEQ ID NO:36. In some embodiments, the V_{L} sequence comprises SEQ ID NO:36.

In some embodiments, the antibody or antigen-binding fragment comprises:
(a) a V_{H} sequence comprising SEQ ID NO:27 and a V_{L} sequence comprising SEQ ID NO:28; or
(b) a V_{H} sequence comprising SEQ ID NO:35 and a V_{L} sequence comprising SEQ ID NO:36; or
(c) a V_{H} sequence comprising SEQ ID NO:37 and a V_{L} sequence comprising SEQ ID NO:36; or
(d) a V_{H} sequence comprising SEQ ID NO:38 and a V_{L} sequence comprising SEQ ID NO:36; or
(e) a V_{H} sequence comprising SEQ ID NO:40 and a V_{L} sequence comprising SEQ ID NO:36; or
(f) a V_{H} sequence comprising SEQ ID NO:42 and a V_{L} sequence comprising SEQ ID NO:36; or
(g) a V_{H} sequence comprising SEQ ID NO:44 and a V_{L} sequence comprising SEQ ID NO:36; or
(h) a V_{H} sequence comprising SEQ ID NO:45 and a V_{L} sequence comprising SEQ ID NO:36; or
(i) a V_{H} sequence comprising SEQ ID NO:46 and a V_{L} sequence comprising SEQ ID NO:36.

In some embodiments, an antibody or antigen-binding fragment thereof that specifically binds to TREM2 comprises:
(a) a CDR-H1 sequence comprising the sequence of G-F-T-F-T-B-F-Y-M-S (SEQ ID NO:48);
(b) a CDR-H2 sequence comprising the sequence of V-I-R-N-X₅-X₆-N-X₈—Y-T-X₁₁-X₁₂-Y-N-P-S-V-K-G (SEQ ID NO:49), wherein X₅ is K or R; X₆ is A or P; X₈ is G or A; X₁₁ is A or T; and X₁₂ is G or D;
(c) a CDR-H3 sequence comprising the sequence of X₁—R-L-X₄—Y-G-F-D-Y (SEQ ID NO:50), wherein X₁ is A or T; and X₄ is T or S;
(d) a CDR-L1 sequence comprising the sequence of Q-S-S-K-S-L-L-H-S-X₁₀-G-K-T-Y-L-N(SEQ ID NO:51), wherein X₁₀ is N or T;
(e) a CDR-L2 sequence comprising the sequence of WMSTRAS (SEQ ID NO:8); and
(f) a CDR-L3 sequence comprising the sequence of Q-Q-F-L-E-X₆-P-F-T (SEQ ID NO:52), wherein X₆ is Y or F.

In some embodiments, the CDR-H1 sequence is selected from any one of SEQ ID NOS:4 and 12. In some embodiments, the CDR-H2 sequence is selected from any one of SEQ ID NOS:5, 13, and 25. In some embodiments, the CDR-H3 sequence is selected from any one of SEQ ID NOS:6, 14, and 17. In some embodiments, the CDR-L1 sequence is selected from any one of SEQ ID NOS:7 and 23. In some embodiments, the CDR-L3 sequence is selected from any one of SEQ ID NOS:9 and 18.

In some embodiments, the antibody or antigen-binding fragment comprises:
(a) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO: 18; or
(b) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO:18; or
(c) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:18; or
(d) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO:18; or
(e) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:6, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(f) a CDR-H1 comprising the amino acid sequence of SEQ ID NO:12, a CDR-H2 comprising the amino acid sequence of SEQ ID NO:13, a CDR-H3 comprising the amino acid sequence of SEQ ID NO:14, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(g) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO: 17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9.

In some embodiments, the antibody or antigen-binding fragment comprises a V_{H} sequence that has at least 85% sequence identity to any one of SEQ ID NOS:2, 10, 15, 19, 21, 24, 26, and 79. In some embodiments, the V_{H} sequence has at least 90% sequence identity to SEQ ID NO:15. In some embodiments, the V_{H} sequence has at least 95% sequence identity to SEQ ID NO:15. In some embodiments, the V_{H} sequence comprises SEQ ID NO:15. In some embodiments, the V_{H} sequence has at least 90% sequence identity to SEQ ID NO:24. In some embodiments, the V_{H} sequence has at least 95% sequence identity to SEQ ID NO:24. In some embodiments, the V_{H} sequence comprises SEQ ID NO:24. In some embodiments, the V_{H} sequence has at least 90% sequence identity to SEQ ID NO:79. In some embodiments, the V_{H} sequence has at least 95% sequence identity to SEQ ID NO:79. In some embodiments, the V_{H} sequence comprises SEQ ID NO:79.

In some embodiments, the antibody or antigen-binding fragment comprises a V_{L} sequence that has at least 85% sequence identity to any one of SEQ ID NOS:3, 11, 16, 20, 22, and 68. In some embodiments, the V_{L} sequence has at least 90% sequence identity to SEQ ID NO:16. In some embodiments, the V_{L} sequence has at least 95% sequence identity to SEQ ID NO:16. In some embodiments, the V_{L} sequence comprises SEQ ID NO:16. In some embodiments, the V_{L} sequence has at least 90% sequence identity to SEQ ID NO:22. In some embodiments, the V_{L} sequence has at least 95% sequence identity to SEQ ID NO:22. In some embodiments, the V_{L} sequence comprises SEQ ID NO:22. In some embodiments, the V_{L} sequence has at least 90% sequence identity to SEQ ID NO:68. In some embodiments, the V_{L} sequence has at least 95% sequence identity to SEQ ID NO:68. In some embodiments, the V_{L} sequence comprises SEQ ID NO:68.

In some embodiments, the antibody or antigen-binding fragment comprises:
(a) a V_{H} sequence comprising SEQ ID NO:15 and a V_{L} sequence comprising SEQ ID NO:16; or
(b) a V_{H} sequence comprising SEQ ID NO:19 and a V_{L} sequence comprising SEQ ID NO:20; or
(c) a V_{H} sequence comprising SEQ ID NO:21 and a V_{L} sequence comprising SEQ ID NO:20; or
(d) a V_{H} sequence comprising SEQ ID NO:19 and a V_{L} sequence comprising SEQ ID NO:22; or
(e) a V_{H} sequence comprising SEQ ID NO:79 and a V_{L} sequence comprising SEQ ID NO:22; or
(f) a V_{H} sequence comprising SEQ ID NO:24 and a V_{L} sequence comprising SEQ ID NO:20; or
(g) a V_{H} sequence comprising SEQ ID NO:26 and a V_{L} sequence comprising SEQ ID NO:20; or
(h) a V_{H} sequence comprising SEQ ID NO:24 and a V_{L} sequence comprising SEQ ID NO:22; or
(i) a V_{H} sequence comprising SEQ ID NO:26 and a V_{L} sequence comprising SEQ ID NO:22; or
(j) a V_{H} sequence comprising SEQ ID NO:2 and a V_{L} sequence comprising SEQ ID NO:3; or
(k) a V_{H} sequence comprising SEQ ID NO:10 and a V_{L} sequence comprising SEQ ID NO:11; or
(l) a V_{H} sequence comprising SEQ ID NO:24 and a V_{L} sequence comprising SEQ ID NO:68.

In some embodiments, an antibody or antigen-binding fragment thereof that specifically binds to TREM2 comprises:
(a) a CDR-H1 sequence comprising the amino acid sequence of any one of SEQ ID NOS:4, 12, and 29;
(b) a CDR-H2 sequence comprising the amino acid sequence of any one of SEQ ID NOS:5, 13, 25, 30, 39, 41, and 43;
(c) a CDR-H3 sequence comprising the amino acid sequence of any one of SEQ ID NOS:6, 14, 17, and 31;
(d) a CDR-L1 sequence comprising the amino acid sequence of any one of SEQ ID NOS:7, 23, and 32;
(e) a CDR-L2 sequence comprising the amino acid sequence of any one of SEQ ID NOS:8 and 33; and
(f) a CDR-L3 sequence comprising the amino acid sequence of any one of SEQ ID NOS:9, 18, and 34.

In some embodiments, the antibody or antigen-binding fragment comprises:
(a) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:6, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(b) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:18; or
(c) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO:18; or
(d) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:18; or
(e) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO:18; or
(f) a CDR-H1 comprising the amino acid sequence of SEQ ID NO:12, a CDR-H2 comprising the amino acid sequence of SEQ ID NO:13, a CDR-H3 comprising the amino acid sequence of SEQ ID NO:14, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9;
(g) a CDR-H1 comprising the amino acid sequence of SEQ ID NO:29, a CDR-H2 comprising the amino acid sequence of SEQ ID NO:30, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(h) a CDR-H1 comprising the amino acid sequence of SEQ ID NO:29, a CDR-H2 comprising the amino acid sequence of SEQ ID NO:39, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(i) a CDR-H1 comprising the amino acid sequence of SEQ ID NO:29, a CDR-H2 comprising the amino acid sequence of SEQ ID NO:41, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(j) a CDR-H1 comprising the amino acid sequence of SEQ ID NO:29, a CDR-H2 comprising the amino acid sequence of SEQ ID NO:43, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(k) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO: 17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9.

In some embodiments, the antibody or antigen-binding fragment comprises a heavy chain variable region that has at least 85% sequence identity to any one of SEQ ID NOS:2, 10, 15, 19, 21, 24, 26, 27, 35, 37, 38, 40, 42, 44, 45, 46, and 79. In some embodiments, the antibody or antigen-binding fragment comprises a light chain variable region that has at least 85% sequence identity to any one of SEQ ID NOS:3, 11, 16, 20, 22, 28, 36, and 68.

In some embodiments, the antibody or antigen-binding fragment comprises:
(a) a V_{H} sequence that has at least 85% sequence identity to SEQ ID NO:2 and a V_{L} sequence that has at least 85% sequence identity to SEQ ID NO:3; or
(b) a V_{H} sequence that has at least 85% sequence identity to SEQ ID NO:10 and a V_{L} sequence that has at least 85% sequence identity to SEQ ID NO:11; or
(c) a V_{H} sequence that has at least 85% sequence identity to SEQ ID NO:15 and a V_{L} sequence that has at least 85% sequence identity to SEQ ID NO:16; or
(d) a V_{H} sequence that has at least 85% sequence identity to SEQ ID NO:19 and a V_{L} sequence that has at least 85% sequence identity to SEQ ID NO:20; or
(e) a V_{H} sequence that has at least 85% sequence identity to SEQ ID NO:21 and a V_{L} sequence that has at least 85% sequence identity to SEQ ID NO:20; or
(f) a V_{H} sequence that has at least 85% sequence identity to SEQ ID NO:19 and a V_{L} sequence that has at least 85% sequence identity to SEQ ID NO:22; or
(g) a V_{H} sequence that has at least 85% sequence identity to SEQ ID NO:79 and a V_{L} sequence that has at least 85% sequence identity to SEQ ID NO:22; or
(h) a V_{H} sequence that has at least 85% sequence identity to SEQ ID NO:24 and a V_{L} sequence that has at least 85% sequence identity to SEQ ID NO:20; or
(i) a V_{H} sequence that has at least 85% sequence identity to SEQ ID NO:26 and a V_{L} sequence that has at least 85% sequence identity to SEQ ID NO:20; or
(j) a V_{H} sequence that has at least 85% sequence identity to SEQ ID NO:24 and a V_{L} sequence that has at least 85% sequence identity to SEQ ID NO:22; or
(k) a V_{H} sequence that has at least 85% sequence identity to SEQ ID NO:26 and a V_{L} sequence that has at least 85% sequence identity to SEQ ID NO:22; or
(l) a V_{H} sequence that has at least 85% sequence identity to SEQ ID NO:27 and a V_{L} sequence that has at least 85% sequence identity to SEQ ID NO:28; or
(m) a V_{H} sequence that has at least 85% sequence identity to SEQ ID NO:35 and a V_{L} sequence that has at least 85% sequence identity to SEQ ID NO:36; or
(n) a V_{H} sequence that has at least 85% sequence identity to SEQ ID NO:37 and a V_{L} sequence that has at least 85% sequence identity to SEQ ID NO:36; or
(o) a V_{H} sequence that has at least 85% sequence identity to SEQ ID NO:38 and a V_{L} sequence that has at least 85% sequence identity to SEQ ID NO:36; or
(p) a V_{H} sequence that has at least 85% sequence identity to SEQ ID NO:40 and a V_{L} sequence that has at least 85% sequence identity to SEQ ID NO:36; or
(q) a V_{H} sequence that has at least 85% sequence identity to SEQ ID NO:42 and a V_{L} sequence that has at least 85% sequence identity to SEQ ID NO:36; or
(r) a V_{H} sequence that has at least 85% sequence identity to SEQ ID NO:44 and a V_{L} sequence that has at least 85% sequence identity to SEQ ID NO:36; or
(s) a V_{H} sequence that has at least 85% sequence identity to SEQ ID NO:45 and a V_{L} sequence that has at least 85% sequence identity to SEQ ID NO:36; or
(t) a V_{H} sequence that has at least 85% sequence identity to SEQ ID NO:46 and a V_{L} sequence that has at least 85% sequence identity to SEQ ID NO:36; or
(u) a V_{H} sequence that has at least 85% sequence identity to SEQ ID NO:24 and a V_{L} sequence that has at least 85% sequence identity to SEQ ID NO:68.

In some embodiments, an antibody or antigen-binding fragment thereof that specifically binds to TREM2 recognizes an epitope that is the same or substantially the same as the epitope recognized by antibody clone selected from the group consisting of: Clone CL0020306, Clone CL0020188, Clone CL0020188-1, Clone CL0020188-2, Clone CL0020188-3, Clone CL0020188-4, Clone CL0020188-5, Clone CL0020188-6, Clone CL0020188-7, Clone CL0020188-8, Clone CL0020307, Clone CL0020123, Clone CL0020123-1, Clone CL0020123-2, Clone CL0020123-3, Clone CL0020123-4, Clone CL0020123-5, Clone CL0020123-6, Clone CL0020123-7, and Clone CL0020123-8.

In some embodiments, the antibody or antigen-binding fragment recognizes an epitope that is the same or substantially the same as the epitope recognized by an antibody clone selected from the group consisting of: Clone CL0020123, Clone CL0020123-1, Clone CL0020123-2, Clone CL0020123-3, Clone CL0020123-4, Clone CL0020123-5, Clone CL0020123-6, Clone CL0020123-7, and Clone CL0020123-8. In particular embodiments, the antibody or antigen-binding fragment recognizes one or more of the following epitopes in SEQ ID NO:1: (i) amino acid residues 55-63 (GEKGPCQRV (SEQ ID NO:70)), (ii) amino acids 96-107 (TLRNLQPHDAGL (SEQ ID NO:71)), and (iii) amino acid residues 126-129 (VEVL (SEQ ID NO:72)). In another aspect, the disclosure features an isolated antibody or antigen-binding fragment thereof that specifically binds to a human TREM2, wherein the antibody or antigen-binding fragment thereof recognizes an epitope comprising or consisting of one or more of the following epitopes in SEQ ID NO:1: (i) amino acid residues 55-63 (GEKGPCQRV (SEQ ID NO:70)), (ii) amino acids 96-107 (TLRNLQPHDAGL (SEQ ID NO:71)), and (iii) amino acid residues 126-129 (VEVL (SEQ ID NO:72)). In some embodiments, the antibody or antigen-binding fragment recognizes an epitope that is the same or substantially the same as the epitope recognized by an antibody clone selected from the group consisting of: Clone CL0020188, Clone CL0020188-1, Clone CL0020188-2, Clone CL0020188-3, Clone CL0020188-4, Clone CL0020188-5, Clone CL0020188-6, Clone CL0020188-7, Clone CL0020188-8, Clone CL0020307, and Clone CL0020306. In particular embodiments, the antibody or antigen-binding fragment recognizes amino acid residues 143-149 (FPGESES (SEQ ID NO:69)) in SEQ ID NO:1. In another aspect, the disclosure features an isolated antibody or antigen-binding fragment thereof that specifically binds to a human TREM2, wherein the antibody or antigen-binding fragment thereof recognizes an epitope comprising or consisting of amino acid residues 143-149 (FPGESES (SEQ ID NO:69)) in SEQ ID NO:1.

In some embodiments, an antibody or antigen-binding fragment as disclosed herein decreases levels of soluble TREM2 protein (sTREM2). In some embodiments, an antibody or antigen-binding fragment as disclosed herein binds soluble TREM2 protein (sTREM2) in healthy human CSF or cynomolgus CSF with better potency compared to a reference antibody. In some embodiments, the reference antibody is represented by a combination of sequences selected from the group consisting of: SEQ ID NOS:73 and 74; SEQ ID NOS:75 and 76; and SEQ ID NOS:77 and 78. In some embodiments, the potency assay is carried out substantially as described in Example 11. In some embodiments, an antibody or antigen-binding fragment as disclosed herein enhances TREM2 activity. In some embodiments, the antibody or antigen-binding fragment thereof enhances phagocytosis or enhances the migration, differentiation, function, or survival of myeloid cells, microglia, or macrophages. In some embodiments, the antibody or antigen-binding fragment thereof enhances microglia function without increasing neuroinflammation. In some embodiments, the antibody or antigen-binding fragment thereof enhances Syk phosphorylation. In some embodiments, the antibody or antigen-binding fragment thereof enhances Syk phosphorylation in the presence of a TREM2 ligand. In some embodiments, the antibody or antigen-binding fragment thereof exhibits cross-reactivity with a cynomolgus TREM2 protein.

In some embodiments, an antibody or antigen-binding fragment as disclosed herein is a monoclonal antibody. In some embodiments, an antibody or antigen-binding fragment as disclosed herein is a chimeric antibody. In some embodiments, an antibody or antigen-binding fragment as disclosed herein is a humanized antibody. In some embodiments, an antibody or antigen-binding fragment as disclosed herein is a fully human antibody. In some embodiments, an antibody or antigen-binding fragment as disclosed herein is a Fab, a F(ab′)₂, a scFv, or a bivalent scFv.

In another aspect, the disclosure provides antibodies or antigen-binding fragments thereof that competes with an isolated anti-TREM2 antibody as disclosed herein for binding to the human TREM2 protein.

In another aspect, the disclosure provides pharmaceutical compositions comprising an antibody or antigen-binding fragment as disclosed herein that specifically binds to TREM2 and a pharmaceutically acceptable carrier.

In yet another aspect, the disclosure provides kits comprising: an antibody or antigen-binding fragment as disclosed herein that specifically binds to TREM2 or a pharmaceutical composition comprising the anti-TREM2 antibody or antigen-binding fragment; and instructions for use thereof.

In still another aspect, the disclosure provides methods of treating a neurodegenerative disease in a subject. In some embodiments, the method comprises administering to the subject an anti-TREM2 antibody or antigen-binding fragment as disclosed herein or a pharmaceutical composition comprising an anti-TREM2 antibody or antigen-binding fragment as disclosed herein.

In some embodiments, the neurodegenerative disease is selected from the group consisting of. Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, and tangle only dementia.

In yet another aspect, the disclosure provides methods of decreasing levels of sTREM2 in a subject having a neurodegenerative disease. In some embodiments, the method comprises administering to the subject an anti-TREM2 antibody or antigen-binding fragment as disclosed herein or a pharmaceutical composition comprising an anti-TREM2 antibody or antigen-binding fragment as disclosed herein.

In still another aspect, the disclosure provides methods of enhancing TREM2 activity in a subject having a neurodegenerative disease. In some embodiments, the method comprises administering to the subject an anti-TREM2 antibody or antigen-binding fragment as disclosed herein or a pharmaceutical composition comprising an anti-TREM2 antibody or antigen-binding fragment as disclosed herein.

### BRIEF DESCRIPTION OF THE DRAWINGS

FIG. 1 includes representative flow cytometry histograms representing binding of an exemplary anti-TREM2 antibody to surface TREM2 on HEK cells expressing TREM2.

FIG. 2 includes a representative dose-response curve of pSyk signal activation by an exemplary anti-TREM2 antibody in primary human macrophage cells. Solid black circles (●) represent anti-TREM2 antibody, and open white circles (°) represent isotype control.

FIGS. 3A and 3B include representative dose-response curves of pSyk signal activation in human iPSC microglia cells after pre-treatment with exemplary anti-TREM2 antibodies for 5 minutes (FIG. 3A) or for 24 hours (FIG. 3B), followed by dosing with lipid vesicles and assessment of liposome response in the cells.

FIG. 4 includes representative dose-response curves of NFAT-luciferase reporter activity in Jurkat NFAT cells expressing human TREM2/DAP12 in response to stimulation by exemplary anti-TREM2 antibodies. Solid black circles (●) represent anti-TREM2 antibody, and open white circles (°) represent isotype control.

FIG. 5 illustrates representative dose-response curves of cell survival in human macrophage cells in response to treatment with exemplary anti-TREM2 antibodies.

FIG. 6 illustrates representative soluble TREM2 levels (sTREM2) as a function of the anti-TREM2 antibody concentration for exemplary anti-TREM2 antibodies.

FIG. 7 is a bar chart indicating mean pHrodo fluorescence intensity per cell in human macrophages treated with exemplary anti-TREM2 antibodies.

FIG. 8A is a representative microscopy image of lipid accumulation in iPSC microglia treated with myelin, followed by incubation with exemplary anti-TREM2 antibody or isotype control.

FIG. 8B is a representative bar chart of Nile Red staining (indicating lipid accumulation) of iPSC microglia that were imaged in FIG. 8A.

FIGS. 8C-8F include bar charts illustrating quantified levels of cholesteryl ester species (FIGS. 8C and 8E) and triacylglyceride lipid species (FIGS. 8D and 8F) in iPSC microglia treated with myelin, followed by incubation with exemplary anti-TREM2 antibodies.

FIGS. 8E and 8F represent data for iPSC microglia for which a myelin washout step was included prior to incubation with the exemplary anti-TREM2 antibodies.

FIG. 9 includes representative mouse plasma pharmacokinetic profiles of exemplary anti-TREM2 antibodies.

FIGS. 10A and 10B include bar charts illustrating change in total soluble TREM2 (sTREM2) (FIG. 10A) and antibody-bound TREM2 (FIG. 10B) in mouse plasma for exemplary anti-TREM2 antibodies, which were injected into TREM2 cDNA KI (huTrem2^{KI/KI}) mice.

FIGS. 11A and 11B include dose-response binding curves to human TREM2 in HEK cells for exemplary humanized and sequence-optimized anti-TREM2 antibodies.

FIGS. 12A and 12B include dose-response curves of pSyk signal activation by exemplary humanized and sequence-optimized anti-TREM2 antibodies in HEK293-H6 cells.

FIG. 13 illustrates dose-response curves of cell survival in human macrophage cells in response to treatment with exemplary humanized and sequence-optimized anti-TREM2 antibodies.

FIGS. 14A and 14B include dose-response curves of lipid clearance in iPSC microglia in response to treatment with exemplary humanized and sequence-optimized anti-TREM2 antibodies.

### DETAILED DESCRIPTION

### I. Introduction

TREM2 is a transmembrane receptor that is expressed on the cell surface of microglia, dendritic cells, macrophages, and osteoclasts. Without being bound to a particular theory, it is believed that upon ligand binding, TREM2 forms a signaling complex with a transmembrane adapter protein, DNAX-activating protein 12 (DAP12), which in turn is tyrosine phosphorylated by the protein kinase SRC. It is believed that the activated TREM2/DAP12 signaling complex mediates intracellular signaling by recruiting and phosphorylating kinases such as Syk kinase. TREM2/DAP12 signaling modulates activities such as phagocytosis, cell growth and survival, pro-inflammatory cytokine secretion, and the migration of cells such as microglia and macrophages. TREM2 undergoes regulated intramembrane proteolysis, in which the membrane-associated full-length TREM2 is cleaved by the metalloprotease ADAM10 into a sTREM2 portion that is shed from the cell and a membrane-retained C-terminal fragment that is further degraded by a gamma-secretase. Altered levels of sTREM2 have been reported in patients having Alzheimer's disease or frontotemporal dementia and having a mutation in TREM2. Additionally, mutations in TREM2 are associated with altered functions such as impaired phagocytosis and reduced microglial function.

As detailed in the Examples section below, antibodies have been generated that specifically bind to human TREM2 and that modulate one or more downstream functions of the TREM2/DAP12 signaling complex. Accordingly, in one aspect, the present disclosure provides anti-TREM2 antibodies and antigen-binding fragments thereof. Accordingly, in one aspect, the present disclosure provides anti-TREM2 antibodies and antigen-binding portions thereof.

In some embodiments, the anti-TREM2 antibodies enhance TREM2 activity, e.g., enhance phagocytosis or enhance the differentiation, function, migration, or survival of myeloid cells, microglia, or macrophages. Thus, in another aspect, methods of enhancing TREM2 activity, e.g., in a subject having a neurodegenerative disease, are provided.

In some embodiments, the anti-TREM2 antibodies reduce shedding of sTREM2. Thus, in another aspect, methods of decreasing levels of sTREM2, e.g., in a subject having a neurodegenerative disease, are provided.

### II. Definitions

As used herein, the singular forms “a,” “an” and “the” include plural referents unless the content clearly dictates otherwise. Thus, for example, reference to “an antibody” optionally includes a combination of two or more such molecules, and the like.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

As used herein, the term “TREM2 protein” refers to a triggering receptor expressed on myeloid cells 2 protein that is encoded by the gene TREM2. As used herein, a “TREM2 protein” refers to a native (i.e., wild-type) TREM2 protein of any vertebrate, such as but not limited to human, non-human primates (e.g., cynomolgus monkey), rodents (e.g., mice, rat), and other mammals. In some embodiments, a TREM2 protein is a human TREM2 protein having the sequence identified in UniprotKB accession number Q9NZC2 (SEQ ID NO:1).

As used herein, the term “anti-TREM2 antibody” refers to an antibody that specifically binds to a TREM2 protein (e.g., human TREM2).

As used herein, the term “antibody” refers to a protein with an immunoglobulin fold that specifically binds to an antigen via its variable regions. The term encompasses intact polyclonal antibodies, intact monoclonal antibodies, single chain antibodies, multispecific antibodies such as bispecific antibodies, monospecific antibodies, monovalent antibodies, chimeric antibodies, humanized antibodies, and human antibodies. The term “antibody,” as used herein, also includes antibody fragments that retain binding specificity via its variable regions, including but not limited to Fab, F(ab′)₂, Fv, scFv, and bivalent scFv. Antibodies can contain light chains that are classified as either kappa or lambda. Antibodies can contain heavy chains that are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (V_{H}) refer to these light and heavy chains, respectively.

The term “variable region” or “variable domain” refers to a domain in an antibody heavy chain or light chain that is derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The term “complementarity determining region” or “CDR” refers to the three hypervariable regions in each chain that interrupt the four framework regions established by the light and heavy chain variable regions. The CDRs are primarily responsible for antibody binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 or CDR-H3 is located in the variable region of the heavy chain of the antibody in which it is found, whereas a V_{L} CDR1 or CDR-L1 is the CDR1 from the variable region of the light chain of the antibody in which it is found.

The “framework regions” or “FRs” of different light or heavy chains are relatively conserved within a species. The framework region of an antibody, that is the combined framework regions of the constituent light and heavy chains, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well-known definitions in the art, e.g., Kabat, Chothia, international ImMunoGeneTics database (IMGT), AbM, and observed antigen contacts (“Contact”). In some embodiments, CDRs are determined according to the Contact definition. See, MacCallum et al., J. Mol. Biol., 262:732-745 (1996). In some embodiments, CDRs are determined by a combination of Kabat, Chothia, and/or Contact CDR definitions.

The terms “antigen-binding portion” and “antigen-binding fragment” are used interchangeably herein and refer to one or more fragments of an antibody that retains the ability to specifically bind to an antigen (e.g., a TREM2 protein) via its variable region. Examples of antigen-binding fragments include, but are not limited to, a Fab fragment (a monovalent fragment consisting of the VL, V_{H}, CL and CH1 domains), F(ab′)₂ fragment (a bivalent fragment comprising two Fab fragments linked by a disulfide bridge at the hinge region), single chain Fv (scFv), disulfide-linked Fv (dsFv), complementarity determining regions (CDRs), a V_{L} (light chain variable region), and a V_{H} (heavy chain variable region).

The term “epitope” refers to the area or region of an antigen to which the CDRs of an antibody specifically binds and can include a few amino acids or portions of a few amino acids, e.g., 5 or 6, or more, e.g., 20 or more amino acids, or portions of those amino acids. For example, where the target is a protein, the epitope can be comprised of consecutive amino acids (e.g., a linear epitope), or amino acids from different parts of the protein that are brought into proximity by protein folding (e.g., a discontinuous or conformational epitope). In some embodiments, the epitope is phosphorylated at one amino acid (e.g., at a serine or threonine residue).

As used herein, the phrase “recognizes an epitope,” as used with reference to an anti-TREM2 antibody, means that the antibody CDRs interact with or specifically bind to the antigen (i.e., the TREM2 protein) at that epitope or a portion of the antigen containing that epitope.

As used herein, the term “multispecific antibody” refers to an antibody that comprises two or more different antigen-binding portions, in which each antigen-binding portion comprises a different variable region that recognizes a different antigen, or a fragment or portion of the antibody that binds to the two or more different antigens via its variable regions. As used herein, the term “bispecific antibody” refers to an antibody that comprises two different antigen-binding portions, in which each antigen-binding portion comprises a different variable region that recognizes a different antigen, or a fragment or portion of the antibody that binds to the two different antigens via its variable regions.

A “monoclonal antibody” refers to antibodies produced by a single clone of cells or a single cell line and consisting of or consisting essentially of antibody molecules that are identical in their primary amino acid sequence.

A “polyclonal antibody” refers to an antibody obtained from a heterogeneous population of antibodies in which different antibodies in the population bind to different epitopes of an antigen.

A “chimeric antibody” refers to an antibody molecule in which the constant region, or a portion thereof, is altered, replaced or exchanged so that the antigen-binding site (i.e., variable region, CDR, or portion thereof) is linked to a constant region of a different or altered class, effector function and/or species, or in which the variable region, or a portion thereof, is altered, replaced or exchanged with a variable region having a different or altered antigen specificity (e.g., CDR and framework regions from different species). In some embodiments, a chimeric antibody is a monoclonal antibody comprising a variable region from one source or species (e.g., mouse) and a constant region derived from a second source or species (e.g., human). Methods for producing chimeric antibodies are described in the art.

A “humanized antibody” is a chimeric immunoglobulin derived from a non-human source (e.g., murine) that contains minimal sequences derived from the non-human immunoglobulin outside the CDRs. In general, a humanized antibody will comprise at least one (e.g., two) antigen-binding variable domain(s), in which the CDR regions substantially correspond to those of the non-human immunoglobulin and the framework regions substantially correspond to those of a human immunoglobulin sequence. The humanized antibody can also comprise at least a portion of an immunoglobulin constant region (Fc), typically that of a human immunoglobulin sequence. Methods of antibody humanization are known in the art.

A “human antibody” or a “fully human antibody” is an antibody having human heavy chain and light chain sequences, typically derived from human germline genes. In some embodiments, the antibody is produced by a human cell, by a non-human animal that utilizes human antibody repertoires (e.g., transgenic mice that are genetically engineered to express human antibody sequences), or by phage display platforms.

The term “specifically binds” refers to a molecule (e.g., an antibody or an antigen-binding portion thereof) that binds to an epitope or target with greater affinity, greater avidity, and/or greater duration to that epitope or target in a sample than it binds to another epitope or non-target compound (e.g., a structurally different antigen). In some embodiments, an antibody (or an antigen-binding portion thereof) that specifically binds to an epitope or target is an antibody (or an antigen-binding portion thereof) that binds to the epitope or target with at least 5-fold greater affinity than other epitopes or non-target compounds, e.g., at least 6-fold, 7-fold, 8-fold, 9-fold, 10-fold, 20-fold, 25-fold, 50-fold, 100-fold, 1,000-fold, 10,000-fold, or greater affinity. The term “specific binding,” “specifically binds to,” or “is specific for” a particular epitope or target, as used herein, can be exhibited, for example, by a molecule having an equilibrium dissociation constant K_{D} for the epitope or target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. It will be recognized by one of skill that an antibody that specifically binds to a target (e.g., a TREM2 protein) from one species may also specifically bind to orthologs of that target (e.g., the TREM2 protein).

The term “binding affinity” is used herein to refer to the strength of a non-covalent interaction between two molecules, e.g., between an antibody (or an antigen-binding portion thereof) and an antigen. Thus, for example, the term may refer to 1:1 interactions between an antibody (or an antigen-binding portion thereof) and an antigen, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet platform). As used herein, “binding affinity” includes not only formal binding affinities, such as those reflecting 1:1 interactions between an antibody (or an antigen-binding portion thereof) and an antigen, but also apparent affinities for which K_{D} values are calculated that may reflect avid binding.

The term “cross-reacts,” as used herein, refers to the ability of an antibody to bind to an antigen other than the antigen against which the antibody was raised. In some embodiments, cross-reactivity refers to the ability of an antibody to bind to an antigen from another species than the antigen against which the antibody was raised. As a non-limiting example, an anti-TREM2 antibody as described herein that is raised against a human TREM2 peptide can exhibit cross-reactivity with a TREM2 peptide or protein from a different species (e.g., monkey or mouse).

The term “isolated,” as used with reference to a nucleic acid or protein (e.g., antibody), denotes that the nucleic acid or protein is essentially free of other cellular components with which it is associated in the natural state. Purity and homogeneity are typically determined using analytical chemistry techniques such as electrophoresis (e.g., polyacrylamide gel electrophoresis) or chromatography (e.g., high performance liquid chromatography). In some embodiments, an isolated nucleic acid or protein (e.g., antibody) is at least 85% pure, at least 90% pure, at least 95% pure, or at least 99% pure.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof “Amino acid analogs” refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions.

The terms “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The terms “conservative substitution” and “conservative mutation” refer to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window, or designated region as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the subject, individual, or patient is a human.

The terms “treating,” “treatment,” and the like are used herein to generally mean obtaining a desired pharmacologic and/or physiologic effect. “Treating” or “treatment” may refer to any indicia of success in the treatment or amelioration of a neurodegenerative disease (e.g., Alzheimer's disease or another neurodegenerative disease described herein), including any objective or subjective parameter such as abatement, remission, improvement in patient survival, increase in survival time or rate, diminishing of symptoms or making the disease more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutically acceptable excipient” refers to a non-active pharmaceutical ingredient that is biologically or pharmacologically compatible for use in humans or animals, such as, but not limited to a buffer, carrier, or preservative.

As used herein, a “therapeutic amount” or “therapeutically effective amount” of an agent (e.g., an antibody as described herein) is an amount of the agent that treats, alleviates, abates, or reduces the severity of symptoms of a disease in a subject. A “therapeutic amount” of an agent (e.g., an antibody as described herein) may improve patient survival, increase survival time or rate, diminish symptoms, make an injury, disease, or condition (e.g., a neurodegenerative disease) more tolerable, slow the rate of degeneration or decline, or improve a patient's physical or mental well-being.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, an antibody as described herein is administered intravenously.

The term “control” or “control value” refers to a reference value or baseline value. Appropriate controls can be determined by one skilled in the art. In some instances, control values can be determined relative to a baseline within the same subject or experiment, e.g., a measurement of sTREM2 taken prior to treatment with an anti-TREM2 antibody can be a control value for a post-treatment measurement of sTREM2 levels in the same subject. In other instances, the control value can be determined relative to a control subject (e.g., a healthy control or a disease control) or an average value in a population of control subjects (e.g., healthy controls or disease controls, e.g., a population of 10, 20, 50, 100, 200, 500, 1000 control subjects or more), e.g, a measurement of a subject's level of sTREM2 either at baseline or after treatment can be compared to a healthy control value.

### III. Anti-TREM2 Antibodies

In one aspect, antibodies and antigen-binding fragments thereof that specifically bind to a TREM2 protein are provided. In some embodiments, the antibody specifically binds to a human TREM2 protein. In some embodiments, an anti-TREM2 antibody is selective for TREM2 over other TREM-like receptors (e.g., TREM1).

In some embodiments, an anti-TREM2 antibody is an antibody that comprises one or more complementarity determining region (CDR), heavy chain variable region, and/or light chain variable region sequences as disclosed herein. In some embodiments, an anti-TREM2 antibody comprises one or more CDR, heavy chain variable region, and/or light chain variable region sequences as disclosed herein and further comprises one or more functional characteristics as disclosed herein, e.g., an antibody that enhances TREM2 activity (e.g., enhances phagocytosis, or enhances the migration, differentiation, function, or survival of a cell such as a myeloid cell, microglia, or macrophage) or an antibody that decreases levels of sTREM2.

### Anti-TREM2 Antibody Sequences

In some embodiments, an anti-TREM2 or antigen-binding fragment thereof comprises a heavy chain sequence, or a portion thereof, and/or a light chain sequence, or a portion thereof, derived from any of the following anti-TREM2 antibodies described herein: Clone CL0020306, Clone CL0020188, Clone CL0020307, and Clone CL0020123. The CDR, heavy chain variable region, and light chain variable region amino acid sequences of these clones is set forth in the Informal Sequence Listing. In some embodiments, the anti-TREM2 antibody is a chimeric antibody. In some embodiments, the anti-TREM2 antibody is a humanized and/or affinity matured antibody.

In some embodiments, an anti-TREM2 antibody comprises one or more CDRs selected from the group consisting of:
(a) a heavy chain CDR1 (CDR-H1) sequence having at least 90% sequence identity to the amino acid sequence of any one of SEQ ID NOS:4, 12, and 29 or having up to two amino acid substitutions relative to the amino acid sequence of any one of SEQ ID NOS:4, 12, and 29;
(b) a heavy chain CDR2 (CDR-H2) sequence having at least 90% sequence identity to the amino acid sequence of any one of SEQ ID NOS:5, 13, 25, 30, 39, 41, and 43 or having up to two amino acid substitutions relative to the amino acid sequence of any one of SEQ ID NOS:5, 13, 25, 30, 39, 41, and 43;
(c) a heavy chain CDR3 (CDR-H3) sequence having at least 90% sequence identity to the amino acid sequence of any one of SEQ ID NOS:6, 14, 17, and 31 or having up to two amino acid substitutions relative to the amino acid sequence of any one of SEQ ID NOS:6, 14, 17, and 31;
(d) a light chain CDR1 (CDR-L1) sequence having at least 90% sequence identity to the amino acid sequence of any one of SEQ ID NOS:7, 23, and 32 or having up to two amino acid substitutions relative to the amino acid sequence of any one of SEQ ID NOS:7, 23, and 32;
(e) a light chain CDR2 (CDR-L2) sequence having at least 90% sequence identity to the amino acid sequence of any one of SEQ ID NOS:8 and 33 or having up to two amino acid substitutions relative to the amino acid sequence of any one of SEQ ID NOS:8 and 33; and
(f) a light chain CDR3 (CDR-L3) sequence having at least 90% sequence identity to the amino acid sequence of any one of SEQ ID NOS:9, 18, and 34 or having up to two amino acid substitutions relative to the amino acid sequence of any one of SEQ ID NOS:9, 18, and 34.

In some embodiments, an anti-TREM2 antibody comprises two, three, four, five, or all six of (a)-(f). In some embodiments, an anti-TREM2 antibody comprises the CDR-H1 of (a), the CDR-H2 of (b), and the CDR-H3 of (c). In some embodiments, an anti-TREM2 antibody comprises the CDR-L1 of (d), the CDR-L2 of (e), and the CDR-L3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, an anti-TREM2 antibody comprises one or more CDRs selected from the group consisting of:
(a) a CDR-H1 sequence comprising the amino acid sequence of any one of SEQ ID NOS:4, 12, and 29;
(b) a CDR-H2 sequence comprising the amino acid sequence of any one of SEQ ID NOS:5, 13, 25, 30, 39, 41, and 43;
(c) a CDR-H3 sequence comprising the amino acid sequence of any one of SEQ ID NOS:6, 14, 17, and 31;
(d) a CDR-L1 sequence comprising the amino acid sequence of any one of SEQ ID NOS:7, 23, and 32;
(e) a CDR-L2 sequence comprising the amino acid sequence of any one of SEQ ID NOS:8 and 33; and
(f) a CDR-L3 sequence comprising the amino acid sequence of any one of SEQ ID NOS:9, 18, and 34.

In some embodiments, an anti-TREM2 antibody comprises two, three, four, five, or all six of (a)-(f). In some embodiments, an anti-TREM2 antibody comprises the CDR-H1 of
(a), the CDR-H2 of (b), and the CDR-H3 of (c). In some embodiments, an anti-TREM2 antibody comprises the CDR-L1 of (d), the CDR-L2 of (e), and the CDR-L3 of (f).

In some embodiments, an anti-TREM2 antibody comprises:
(a) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:6, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(b) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:18; or
(c) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO:18; or
(d) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:18; or
(e) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO:18; or
(f) a CDR-H1 comprising the amino acid sequence of SEQ ID NO:12, a CDR-H2 comprising the amino acid sequence of SEQ ID NO:13, a CDR-H3 comprising the amino acid sequence of SEQ ID NO:14, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(g) a CDR-H1 comprising the amino acid sequence of SEQ ID NO:29, a CDR-H2 comprising the amino acid sequence of SEQ ID NO:30, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(h) a CDR-H1 comprising the amino acid sequence of SEQ ID NO:29, a CDR-H2 comprising the amino acid sequence of SEQ ID NO:39, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(i) a CDR-H1 comprising the amino acid sequence of SEQ ID NO:29, a CDR-H2 comprising the amino acid sequence of SEQ ID NO:41, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(j) a CDR-H1 comprising the amino acid sequence of SEQ ID NO:29, a CDR-H2 comprising the amino acid sequence of SEQ ID NO:43, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34.

In some embodiments, an anti-TREM2 antibody comprises a heavy chain variable region comprising an amino acid sequence that has at least 85%, 90%, 91%, 92%, 93%, 94%, 95%, 96%, 97%, 98%, or 99% sequence identity to any one of SEQ ID NOS:2, 10, 15, 19, 21, 24, 26, 27, 35, 37, 38, 40, 42, 44, 45, 46, and 79. In some embodiments, an anti-TREM2 comprises a heavy chain variable region comprising the amino acid sequence of any one of SEQ ID NOS:2, 10, 15, 19, 21, 24, 26, 27, 35, 37, 38, 40, 42, 44, 45, 46, and 79.

In some embodiments, an anti-TREM2 antibody comprises a light chain variable region comprising an amino acid sequence that has at least 85%, 90%, 91%, 92%, 93%, 94%, 95%, 96%, 97%, 98%, or 99% sequence identity to any one of SEQ ID NOS:3, 11, 16, 20, 22, 28, 36, and 68. In some embodiments, an anti-TREM2 antibody comprises a light chain variable region comprising the amino acid sequence of any one of SEQ ID NOS:3, 11, 16, 20, 22, 28, 36, and 68.

In some embodiments, an anti-TREM2 antibody comprises: a heavy chain variable region comprising an amino acid sequence that has at least 85%, 90%, 91%, 92%, 93%, 94%, 95%, 96%, 97%, 98%, or 99% sequence identity to any one of SEQ ID NOS:2, 10, 15, 19, 21, 24, 26, 27, 35, 37, 38, 40, 42, 44, 45, 46, and 79, and a light chain variable region comprising an amino acid sequence that has at least 85%, 90%, 91%, 92%, 93%, 94%, 95%, 96%, 97%, 98%, or 99% sequence identity to any one of SEQ ID NOS:3, 11, 16, 20, 22, 28, 36, and 68. In some embodiments, an anti-TREM2 comprises: a heavy chain variable region comprising the amino acid sequence of any one of SEQ ID NOS:2, 10, 15, 19, 21, 24, 26, 27, 35, 37, 38, 40, 42, 44, 45, 46, and 79, and a light chain variable region comprising the amino acid sequence of any one of SEQ ID NOS:3, 11, 16, 20, 22, 28, 36, and 68.

In some embodiments, an anti-TREM2 antibody comprises:
(a) a V_{H} sequence that has at least 85% sequence identity to SEQ ID NO:2 and a V_{L} sequence that has at least 85% sequence identity to SEQ ID NO:3; or
(b) a V_{H} sequence that has at least 85% sequence identity to SEQ ID NO:10 and a V_{L} sequence that has at least 85% sequence identity to SEQ ID NO:11; or
(c) a V_{H} sequence that has at least 85% sequence identity to SEQ ID NO:15 and a V_{L} sequence that has at least 85% sequence identity to SEQ ID NO:16; or
(d) a V_{H} sequence that has at least 85% sequence identity to SEQ ID NO:19 and a V_{L} sequence that has at least 85% sequence identity to SEQ ID NO:20; or
(e) a V_{H} sequence that has at least 85% sequence identity to SEQ ID NO:21 and a V_{L} sequence that has at least 85% sequence identity to SEQ ID NO:20; or
(f) a V_{H} sequence that has at least 85% sequence identity to SEQ ID NO:19 and a V_{L} sequence that has at least 85% sequence identity to SEQ ID NO:22; or
(g) a V_{H} sequence that has at least 85% sequence identity to SEQ ID NO:79 and a V_{L} sequence that has at least 85% sequence identity to SEQ ID NO:22; or
(h) a V_{H} sequence that has at least 85% sequence identity to SEQ ID NO:24 and a V_{L} sequence that has at least 85% sequence identity to SEQ ID NO:20; or
(i) a V_{H} sequence that has at least 85% sequence identity to SEQ ID NO:26 and a V_{L} sequence that has at least 85% sequence identity to SEQ ID NO:20; or
(j) a V_{H} sequence that has at least 85% sequence identity to SEQ ID NO:24 and a V_{L} sequence that has at least 85% sequence identity to SEQ ID NO:22; or
(k) a V_{H} sequence that has at least 85% sequence identity to SEQ ID NO:26 and a V_{L} sequence that has at least 85% sequence identity to SEQ ID NO:22; or
(l) a V_{H} sequence that has at least 85% sequence identity to SEQ ID NO:27 and a V_{L} sequence that has at least 85% sequence identity to SEQ ID NO:28; or
(m) a V_{H} sequence that has at least 85% sequence identity to SEQ ID NO:35 and a V_{L} sequence that has at least 85% sequence identity to SEQ ID NO:36; or
(n) a V_{H} sequence that has at least 85% sequence identity to SEQ ID NO:37 and a V_{L} sequence that has at least 85% sequence identity to SEQ ID NO:36; or
(o) a V_{H} sequence that has at least 85% sequence identity to SEQ ID NO:38 and a V_{L} sequence that has at least 85% sequence identity to SEQ ID NO:36; or
(p) a V_{H} sequence that has at least 85% sequence identity to SEQ ID NO:40 and a V_{L} sequence that has at least 85% sequence identity to SEQ ID NO:36; or
(q) a V_{H} sequence that has at least 85% sequence identity to SEQ ID NO:42 and a V_{L} sequence that has at least 85% sequence identity to SEQ ID NO:36; or
(r) a V_{H} sequence that has at least 85% sequence identity to SEQ ID NO:44 and a V_{L} sequence that has at least 85% sequence identity to SEQ ID NO:36; or
(s) a V_{H} sequence that has at least 85% sequence identity to SEQ ID NO:45 and a V_{L} sequence that has at least 85% sequence identity to SEQ ID NO:36; or
(t) a V_{H} sequence that has at least 85% sequence identity to SEQ ID NO:46 and a V_{L} sequence that has at least 85% sequence identity to SEQ ID NO:36; or
(u) a V_{H} sequence that has at least 85% sequence identity to SEQ ID NO:24 and a V_{L} sequence that has at least 85% sequence identity to SEQ ID NO:68.

In some embodiments, an anti-TREM2 antibody comprises one or more sequences that are encompassed by a consensus sequence disclosed herein. As a non-limiting example, consensus sequences can be identified by aligning heavy chain or light chain sequences (e.g., CDRs) for antibodies that are from the same (or similar) germlines. In some embodiments, consensus sequences may be generated from antibodies that contain sequences that are of the same (or similar) length and/or have at least one highly similar CDR (e.g., a highly similar CDR3). In some embodiments, such sequences in these antibodies may be aligned and compared to identify conserved amino acids or motifs (i.e., where alteration in sequences may alter protein function) and/or regions where variation occurs the sequences (i.e., where variation of sequence is not likely to significantly affect protein function). Alternatively, consensus sequences can be identified by aligning heavy chain or light chain sequences (e.g., CDRs) for antibodies that bind to the same or similar (e.g., overlapping) epitopes to determine conserved amino acids or motifs (i.e., where alteration in sequences may alter protein function) and regions where variation occurs in alignment of sequences (i.e., where variation of sequence is not likely to significantly affect protein function). In some embodiments, one or more consensus sequences can be identified for antibodies that recognize the same or similar epitope as an anti-TREM2 antibody as disclosed herein. Exemplary consensus sequences include SEQ ID NOS:47-52. In the consensus sequences of SEQ ID NOS:47-52, the capitalized letter represents an amino acid residue that is absolutely conserved among the aligned sequences (e.g., aligned CDR sequences), while an “X” or a Greek letter (e.g., “α,” “β,” “γ,” “δ,” “ε,” or “φ”) represents an amino acid residue that is not absolutely conserved among the aligned sequences. It will be appreciated that, when selecting an amino acid to insert at a position marked by an “X” or by a Greek letter, in some embodiments the amino acid is selected from those amino acids found at the corresponding position in the aligned sequences.

### Clone CL0020123 and Variants of CL0020123

In some embodiments, an anti-TREM2 antibody or antigen-binding fragment thereof comprises:
(a) a CDR-H1 sequence comprising the sequence of GFSIEDFYIH (SEQ ID NO:29);
(b) a CDR-H2 sequence comprising the sequence of W-I-D-P-E-X₆-G-X₈-S-K-Y-A-P-K-F-Q-G (SEQ ID NO:47), wherein X₆ is N or Q and X₈ is D or E;
(c) a CDR-H3 sequence comprising the sequence of HADHGNYGSTMDY (SEQ ID NO:31);
(d) a CDR-L1 sequence comprising the sequence of HASQHINVWLS (SEQ ID NO:32);
(e) a CDR-L2 sequence comprising the sequence of KASNLHT (SEQ ID NO:33); and
(f) a CDR-L3 sequence comprising the sequence of QQGQTYPRT (SEQ ID NO:34).

In some embodiments, an anti-TREM2 antibody comprises a CDR-H2 sequence that is selected from SEQ ID NOS:30, 39, 41, and 43.

In some embodiments, an anti-TREM2 antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to any one of SEQ ID NOS:27, 35, 37, 38, 40, 42, 44, 45, and 46. In some embodiments, an anti-TREM2 antibody comprises a heavy chain variable region comprising the amino acid sequence of any one of SEQ ID NOS:27, 35, 37, 38, 40, 42, 44, 45, and 46.

In some embodiments, an anti-TREM2 antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to any one of SEQ ID NOS:28 and 36. In some embodiments, an anti-TREM2 antibody comprises a light chain variable region comprising the amino acid sequence of any one of SEQ ID NOS:28 and 36.

In some embodiments, an anti-TREM2 antibody comprises a CDR-H1 sequence comprising the amino acid sequence of SEQ ID NO:29, a CDR-H2 sequence comprising the amino acid sequence of SEQ ID NO:30, a CDR-H3 sequence comprising the amino acid sequence of SEQ ID NO:31, a CDR-L1 sequence comprising the amino acid sequence of SEQ ID NO:32, a CDR-L2 sequence comprising the amino acid sequence of SEQ ID NO:33, and a CDR-L3 sequence comprising the amino acid sequence of SEQ ID NO:34.

In some embodiments, an anti-TREM2 antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:27. In some embodiments, an anti-TREM2 antibody comprises a heavy chain variable region comprising the amino acid sequence of SEQ ID NO:27.

In some embodiments, an anti-TREM2 antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:28. In some embodiments, an anti-TREM2 antibody comprises a light chain variable region comprising the amino acid sequence of SEQ ID NO:28.

In some embodiments, an anti-TREM2 antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:27 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:28. In some embodiments, an anti-TREM2 antibody comprises a heavy chain variable region comprising the amino acid sequence of SEQ ID NO:27 and a light chain variable region comprising the amino acid sequence of SEQ ID NO:28.

In some embodiments, an anti-TREM2 antibody comprises a heavy chain variable region that comprises a heavy chain CDR1-3 comprising the amino acid sequences of SEQ ID NOS:29, 30, and 31, respectively, and that has at least 85% sequence identity (e.g., at least 90%, 91%, 92%, 93%, 94%, 95%, 96%, 97%, 98%, or 99% sequence identity) to SEQ ID NO:27. In some embodiments, an anti-TREM2 antibody comprises a light chain variable region that comprises a light chain CDR1-3 comprising the amino acid sequences of SEQ ID NOS:32, 33, and 34, respectively, and that has at least 85% sequence identity (e.g., at least 90%, 91%, 92%, 93%, 94%, 95%, 96%, 97%, 98%, or 99% sequence identity) to SEQ ID NO:28.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOS:29, 30, 31, 32, 33, and 34, respectively, or an antibody comprising a heavy chain variable region comprising the amino acid sequence of SEQ ID NO:27 and a light chain variable region comprising the amino acid sequence of SEQ ID NO:28).

In some embodiments, an anti-TREM2 antibody comprises a CDR-H1 sequence comprising the amino acid sequence of SEQ ID NO:29, a CDR-H2 sequence comprising the amino acid sequence of SEQ ID NO:43, a CDR-H3 sequence comprising the amino acid sequence of SEQ ID NO:31, a CDR-L1 sequence comprising the amino acid sequence of SEQ ID NO:32, a CDR-L2 sequence comprising the amino acid sequence of SEQ ID NO:33, and a CDR-L3 sequence comprising the amino acid sequence of SEQ ID NO:34.

In some embodiments, an anti-TREM2 antibody comprises a heavy chain variable region comprising an amino acid sequence that has at least 85% sequence identity (e.g., at least 90%, 95%, or 97% sequence identity) to SEQ ID NO:42. In some embodiments, an anti-TREM2 antibody comprises a heavy chain variable region comprising the amino acid sequence of SEQ ID NO:42.

In some embodiments, an anti-TREM2 antibody comprises a light chain variable region comprising an amino acid sequence that has at least 85% sequence identity (e.g., at least 90%, 95%, or 97% sequence identity) to SEQ ID NO:36. In some embodiments, an anti-TREM2 antibody comprises a light chain variable region comprising the amino acid sequence of SEQ ID NO:36.

In some embodiments, an anti-TREM2 antibody comprises a heavy chain variable region comprising an amino acid sequence that has at least 85% sequence identity (e.g., at least 90%, 95%, or 97% sequence identity) to SEQ ID NO:42 and a light chain variable region comprising an amino acid sequence that has at least 85% sequence identity (e.g., at least 90%, 95%, or 97% sequence identity) to SEQ ID NO:36. In some embodiments, an anti-TREM2 antibody comprises a heavy chain variable region comprising the amino acid sequence of SEQ ID NO:42 and a light chain variable region comprising the amino acid sequence of SEQ ID NO:36.

In some embodiments, an anti-TREM2 antibody comprises a heavy chain variable region that comprises a heavy chain CDR1-3 comprising the amino acid sequences of SEQ ID NOs:29, 43, and 31, respectively, and that has at least 85% sequence identity (e.g., at least 90%, 95%, or 97% sequence identity) to SEQ ID NO:42. In some embodiments, an anti-TREM2 antibody comprises a light chain variable region that comprises a light chain CDR1-3 comprising the amino acid sequences of SEQ ID NOs:32, 33, and 34, respectively, and that has at least 85% sequence identity (e.g., at least 90%, 95%, or 97% sequence identity) to SEQ ID NO:36.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOS:29, 43, 31, 32, 33, and 34, respectively, or an antibody comprising a heavy chain variable region comprising the amino acid sequence of SEQ ID NO:42 and a light chain variable region comprising the amino acid sequence of SEQ ID NO:36).

In some embodiments, an anti-TREM2 antibody comprises a CDR-H1 sequence comprising the amino acid sequence of SEQ ID NO:29, a CDR-H2 sequence comprising the amino acid sequence of SEQ ID NO:41, a CDR-H3 sequence comprising the amino acid sequence of SEQ ID NO:31, a CDR-L1 sequence comprising the amino acid sequence of SEQ ID NO:32, a CDR-L2 sequence comprising the amino acid sequence of SEQ ID NO:33, and a CDR-L3 sequence comprising the amino acid sequence of SEQ ID NO:34.

In some embodiments, an anti-TREM2 antibody comprises a heavy chain variable region comprising an amino acid sequence that has at least 85% sequence identity (e.g., at least 90%, 95%, or 97% sequence identity) to SEQ ID NO:45. In some embodiments, an anti-TREM2 antibody comprises a heavy chain variable region comprising the amino acid sequence of SEQ ID NO:45.

In some embodiments, an anti-TREM2 antibody comprises a light chain variable region comprising an amino acid sequence that has at least 85% sequence identity (e.g., at least 90%, 95%, or 97% sequence identity) to SEQ ID NO:36. In some embodiments, an anti-TREM2 antibody comprises a light chain variable region comprising the amino acid sequence of SEQ ID NO:36.

In some embodiments, an anti-TREM2 antibody comprises a heavy chain variable region comprising an amino acid sequence that has at least 85% sequence identity (e.g., at least 90%, 95%, or 97% sequence identity) to SEQ ID NO:45 and a light chain variable region comprising an amino acid sequence that has at least 85% sequence identity (e.g., at least 90%, 95%, or 97% sequence identity) to SEQ ID NO:36. In some embodiments, an anti-TREM2 antibody comprises a heavy chain variable region comprising the amino acid sequence of SEQ ID NO:45 and a light chain variable region comprising the amino acid sequence of SEQ ID NO:36.

In some embodiments, an anti-TREM2 antibody comprises a heavy chain variable region that comprises a heavy chain CDR1-3 comprising the amino acid sequences of SEQ ID NOs:29, 41, and 31, respectively, and that has at least 85% sequence identity (e.g., at least 90%, 95%, or 97% sequence identity) to SEQ ID NO:45. In some embodiments, an anti-TREM2 antibody comprises a light chain variable region that comprises a light chain CDR1-3 comprising the amino acid sequences of SEQ ID NOs:32, 33, and 34, respectively, and that has at least 85% sequence identity (e.g., at least 90%, 95%, or 97% sequence identity) to SEQ ID NO:36.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOS:29, 41, 31, 32, 33, and 34, respectively, or an antibody comprising a heavy chain variable region comprising the amino acid sequence of SEQ ID NO:45 and a light chain variable region comprising the amino acid sequence of SEQ ID NO:36).

### Clones CL0020188, CL0020306, CL0020307, and Variants of CL0020188

In some embodiments, an anti-TREM2 antibody or antigen-binding fragment thereof comprises:
(a) a CDR-H1 sequence comprising the sequence of G-F-T-F-T-B-F-Y-M-S (SEQ ID NO:48);
(b) a CDR-H2 sequence comprising the sequence of V-I-R-N-X₅-X₆-N-X₈—Y-T-X₁₁-X₁₂-Y-N-P-S-V-K-G (SEQ ID NO:49), wherein X₅ is K or R; X₆ is A or P; X₈ is G or A; X₁₁ is A or T; and X₁₂ is G or D;
(c) a CDR-H3 sequence comprising the sequence of X₁—R-L-X₄—Y-G-F-D-Y (SEQ ID NO:50), wherein X₁ is A or T; and X₄ is T or S;
(d) a CDR-L1 sequence comprising the sequence of Q-S-S-K-S-L-L-H-S-X₁₀-G-K-T-Y-L-N(SEQ ID NO:51), wherein X₁₀ is N or T;
(e) a CDR-L2 sequence comprising the sequence of WMSTRAS (SEQ ID NO:8); and
(f) a CDR-L3 sequence comprising the sequence of Q-Q-F-L-E-X₆-P-F-T (SEQ ID NO:52), wherein X₆ is Y or F.

In some embodiments, an anti-TREM2 antibody comprises a CDR-H1 sequence that is selected from SEQ ID NOS:4 and 12. In some embodiments, an anti-TREM2 antibody comprises a CDR-H2 sequence that is selected from SEQ ID NOS:5, 13, and 25. In some embodiments, an anti-TREM2 antibody comprises a CDR-H3 sequence that is selected from SEQ ID NOS:6, 14, and 17. In some embodiments, an anti-TREM2 antibody comprises a CDR-L1 sequence that is selected from SEQ ID NOS:7 and 23. In some embodiments, an anti-TREM2 antibody comprises a CDR-L3 sequence is selected from SEQ ID NOS:9 and 18.

In some embodiments, an anti-TREM2 antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to any one of SEQ ID NOS:2, 10, 15, 19, 21, 24, 26, and 79. In some embodiments, an anti-TREM2 antibody comprises a heavy chain variable region comprising the amino acid sequence of any one of SEQ ID NOS:2, 10, 15, 19, 21, 24, 26, and 79.

In some embodiments, an anti-TREM2 antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to any one of SEQ ID NOS:3, 11, 16, 20, 22, and 68. In some embodiments, an anti-TREM2 antibody comprises a light chain variable region comprising the amino acid sequence of any one of SEQ ID NOS:3, 11, 16, 20, 22, and 68.

### Clone CL0020188 and Variants of CL0020188

In some embodiments, an anti-TREM2 antibody comprises a CDR-H1 sequence comprising the amino acid sequence of SEQ ID NO:4, a CDR-H2 sequence comprising the amino acid sequence of SEQ ID NO:5, a CDR-H3 sequence comprising the amino acid sequence of SEQ ID NO:17, a CDR-L1 sequence comprising the amino acid sequence of SEQ ID NO:7, a CDR-L2 sequence comprising the amino acid sequence of SEQ ID NO:8, and a CDR-L3 sequence comprising the amino acid sequence of SEQ ID NO:18.

In some embodiments, an anti-TREM2 antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:15. In some embodiments, an anti-TREM2 antibody comprises a heavy chain variable region comprising the amino acid sequence of SEQ ID NO: 15.

In some embodiments, an anti-TREM2 antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:16. In some embodiments, an anti-TREM2 antibody comprises a light chain variable region comprising the amino acid sequence of SEQ ID NO:16.

In some embodiments, an anti-TREM2 antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:15 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:16. In some embodiments, an anti-TREM2 antibody comprises a heavy chain variable region comprising the amino acid sequence of SEQ ID NO:15 and a light chain variable region comprising the amino acid sequence of SEQ ID NO:16.

In some embodiments, an anti-TREM2 antibody comprises a heavy chain variable region that comprises a heavy chain CDR1-3 comprising the amino acid sequences of SEQ ID NOS:4, 5, and 17, respectively, and that has at least 85% sequence identity (e.g., at least 90%, 91%, 92%, 93%, 94%, 95%, 96%, 97%, 98%, or 99% sequence identity) to SEQ ID NO:15. In some embodiments, an anti-TREM2 antibody comprises a light chain variable region that comprises a light chain CDR1-3 comprising the amino acid sequences of SEQ ID NOS:7, 8, and 18, respectively, and that has at least 85% sequence identity (e.g., at least 90%, 91%, 92%, 93%, 94%, 95%, 96%, 97%, 98%, or 99% sequence identity) to SEQ ID NO:16.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOS:5, 17, 7, 8, and 18, respectively, or an antibody comprising a heavy chain variable region comprising the amino acid sequence of SEQ ID NO:15 and a light chain variable region comprising the amino acid sequence of SEQ ID NO: 16).

In some embodiments, an anti-TREM2 antibody comprises a CDR-H1 sequence comprising the amino acid sequence of SEQ ID NO:4, a CDR-H2 sequence comprising the amino acid sequence of SEQ ID NO:5, a CDR-H3 sequence comprising the amino acid sequence of SEQ ID NO:17, a CDR-L1 sequence comprising the amino acid sequence of SEQ ID NO:23, a CDR-L2 sequence comprising the amino acid sequence of SEQ ID NO:8, and a CDR-L3 sequence comprising the amino acid sequence of SEQ ID NO:18.

In some embodiments, an anti-TREM2 antibody comprises a heavy chain variable region comprising an amino acid sequence that has at least 85% sequence identity (e.g., at least 90%, 95%, or 97% sequence identity) to SEQ ID NO:79. In some embodiments, an anti-TREM2 antibody comprises a heavy chain variable region comprising the amino acid sequence of SEQ ID NO:79.

In some embodiments, an anti-TREM2 antibody comprises a light chain variable region comprising an amino acid sequence that has at least 85% sequence identity (e.g., at least 90%, 95%, or 97% sequence identity) to SEQ ID NO:22. In some embodiments, an anti-TREM2 antibody comprises a light chain variable region comprising the amino acid sequence of SEQ ID NO:22.

In some embodiments, an anti-TREM2 antibody comprises a heavy chain variable region comprising an amino acid sequence that has at least 85% sequence identity (e.g., at least 90%, 95%, or 97% sequence identity) to SEQ ID NO:79 and a light chain variable region comprising an amino acid sequence that has at least 85% sequence identity (e.g., at least 90%, 95%, or 97% sequence identity) to SEQ ID NO:22. In some embodiments, an anti-TREM2 antibody comprises a heavy chain variable region comprising the amino acid sequence of SEQ ID NO:79 and a light chain variable region comprising the amino acid sequence of SEQ ID NO:22.

In some embodiments, an anti-TREM2 antibody comprises a heavy chain variable region that comprises a heavy chain CDR1-3 comprising the amino acid sequences of SEQ ID NOs:4, 5, and 17, respectively, and that has at least 85% sequence identity (e.g., at least 90%, 95%, or 97% sequence identity) to SEQ ID NO:79. In some embodiments, an anti-TREM2 antibody comprises a light chain variable region that comprises a light chain CDR1-3 comprising the amino acid sequences of SEQ ID NOs:23, 8, and 18, respectively, and that has at least 85% sequence identity (e.g., at least 90%, 95%, or 97% sequence identity) to SEQ ID NO:22.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOS:4, 5, 17, 23, 8, and 18, respectively, or an antibody comprising a heavy chain variable region comprising the amino acid sequence of SEQ ID NO:79 and a light chain variable region comprising the amino acid sequence of SEQ ID NO:22).

In some embodiments, an anti-TREM2 antibody comprises a CDR-H1 sequence comprising the amino acid sequence of SEQ ID NO:4, a CDR-H2 sequence comprising the amino acid sequence of SEQ ID NO:25, a CDR-H3 sequence comprising the amino acid sequence of SEQ ID NO:17, a CDR-L1 sequence comprising the amino acid sequence of SEQ ID NO:23, a CDR-L2 sequence comprising the amino acid sequence of SEQ ID NO:8, and a CDR-L3 sequence comprising the amino acid sequence of SEQ ID NO:18.

In some embodiments, an anti-TREM2 antibody comprises a heavy chain variable region comprising an amino acid sequence that has at least 85% sequence identity (e.g., at least 90%, 95%, or 97% sequence identity) to SEQ ID NO:24. In some embodiments, an anti-TREM2 antibody comprises a heavy chain variable region comprising the amino acid sequence of SEQ ID NO:24.

In some embodiments, an anti-TREM2 antibody comprises a light chain variable region comprising an amino acid sequence that has at least 85% sequence identity (e.g., at least 90%, 95%, or 97% sequence identity) to SEQ ID NO:22. In some embodiments, an anti-TREM2 antibody comprises a light chain variable region comprising the amino acid sequence of SEQ ID NO:22.

In some embodiments, an anti-TREM2 antibody comprises a heavy chain variable region comprising an amino acid sequence that has at least 85% sequence identity (e.g., at least 90%, 95%, or 97% sequence identity) to SEQ ID NO:24 and a light chain variable region comprising an amino acid sequence that has at least 85% sequence identity (e.g., at least 90%, 95%, or 97% sequence identity) to SEQ ID NO:22. In some embodiments, an anti-TREM2 antibody comprises a heavy chain variable region comprising the amino acid sequence of SEQ ID NO:24 and a light chain variable region comprising the amino acid sequence of SEQ ID NO:22.

In some embodiments, an anti-TREM2 antibody comprises a heavy chain variable region that comprises a heavy chain CDR1-3 comprising the amino acid sequences of SEQ ID NOs:4, 25, and 17, respectively, and that has at least 85% sequence identity (e.g., at least 90%, 95%, or 97% sequence identity) to SEQ ID NO:24. In some embodiments, an anti-TREM2 antibody comprises a light chain variable region that comprises a light chain CDR1-3 comprising the amino acid sequences of SEQ ID NOs:23, 8, and 18, respectively, and that has at least 85% sequence identity (e.g., at least 90%, 95%, or 97% sequence identity) to SEQ ID NO:22.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOS:4, 25, 17, 23, 8, and 18, respectively, or an antibody comprising a heavy chain variable region comprising the amino acid sequence of SEQ ID NO:24 and a light chain variable region comprising the amino acid sequence of SEQ ID NO:22).

In some embodiments, an anti-TREM2 antibody comprises a CDR-H1 sequence comprising the amino acid sequence of SEQ ID NO:4, a CDR-H2 sequence comprising the amino acid sequence of SEQ ID NO:25, a CDR-H3 sequence comprising the amino acid sequence of SEQ ID NO:17, a CDR-L1 sequence comprising the amino acid sequence of SEQ ID NO:7, a CDR-L2 sequence comprising the amino acid sequence of SEQ ID NO:8, and a CDR-L3 sequence comprising the amino acid sequence of SEQ ID NO:9.

In some embodiments, an anti-TREM2 antibody comprises a heavy chain variable region comprising an amino acid sequence that has at least 85% sequence identity (e.g., at least 90%, 95%, or 97% sequence identity) to SEQ ID NO:24. In some embodiments, an anti-TREM2 antibody comprises a heavy chain variable region comprising the amino acid sequence of SEQ ID NO:24.

In some embodiments, an anti-TREM2 antibody comprises a light chain variable region comprising an amino acid sequence that has at least 85% sequence identity (e.g., at least 90%, 95%, or 97% sequence identity) to SEQ ID NO:68. In some embodiments, an anti-TREM2 antibody comprises a light chain variable region comprising the amino acid sequence of SEQ ID NO:68.

In some embodiments, an anti-TREM2 antibody comprises a heavy chain variable region comprising an amino acid sequence that has at least 85% sequence identity (e.g., at least 90%, 95%, or 97% sequence identity) to SEQ ID NO:24 and a light chain variable region comprising an amino acid sequence that has at least 85% sequence identity (e.g., at least 90%, 95%, or 97% sequence identity) to SEQ ID NO:68. In some embodiments, an anti-TREM2 antibody comprises a heavy chain variable region comprising the amino acid sequence of SEQ ID NO:24 and a light chain variable region comprising the amino acid sequence of SEQ ID NO:68.

In some embodiments, an anti-TREM2 antibody comprises a heavy chain variable region that comprises a heavy chain CDR1-3 comprising the amino acid sequences of SEQ ID NOs:4, 25, and 17, respectively, and that has at least 85% sequence identity (e.g., at least 90%, 95%, or 97% sequence identity) to SEQ ID NO:24. In some embodiments, an anti-TREM2 antibody comprises a light chain variable region that comprises a light chain CDR1-3 comprising the amino acid sequences of SEQ ID NOs:7, 8, and 9, respectively, and that has at least 85% sequence identity (e.g., at least 90%, 95%, or 97% sequence identity) to SEQ ID NO:68.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOS:4, 25, 17, 7, 8, and 9, respectively, or an antibody comprising a heavy chain variable region comprising the amino acid sequence of SEQ ID NO:24 and a light chain variable region comprising the amino acid sequence of SEQ ID NO:68).

### Clone CL0020306

In some embodiments, an anti-TREM2 antibody comprises a CDR-H1 sequence comprising the amino acid sequence of SEQ ID NO:4, a CDR-H2 sequence comprising the amino acid sequence of SEQ ID NO:5, a CDR-H3 sequence comprising the amino acid sequence of SEQ ID NO:6, a CDR-L1 sequence comprising the amino acid sequence of SEQ ID NO:7, a CDR-L2 sequence comprising the amino acid sequence of SEQ ID NO:8, and a CDR-L3 sequence comprising the amino acid sequence of SEQ ID NO:9.

In some embodiments, an anti-TREM2 antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:2. In some embodiments, an anti-TREM2 antibody comprises a heavy chain variable region comprising the amino acid sequence of SEQ ID NO:2.

In some embodiments, an anti-TREM2 antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:3. In some embodiments, an anti-TREM2 antibody comprises a light chain variable region comprising the amino acid sequence of SEQ ID NO:3.

In some embodiments, an anti-TREM2 antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:2 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:3. In some embodiments, an anti-TREM2 antibody comprises a heavy chain variable region comprising the amino acid sequence of SEQ ID NO:2 and a light chain variable region comprising the amino acid sequence of SEQ ID NO:3.

In some embodiments, an anti-TREM2 antibody comprises a heavy chain variable region that comprises a heavy chain CDR1-3 comprising the amino acid sequences of SEQ ID NOS:4, 5, and 6, respectively, and that has at least 85% sequence identity (e.g., at least 90%, 91%, 92%, 93%, 94%, 95%, 96%, 97%, 98%, or 99% sequence identity) to SEQ ID NO:2. In some embodiments, an anti-TREM2 antibody comprises a light chain variable region that comprises a light chain CDR1-3 comprising the amino acid sequences of SEQ ID NOS:7, 8, and 9, respectively, and that has at least 85% sequence identity (e.g., at least 90%, 91%, 92%, 93%, 94%, 95%, 96%, 97%, 98%, or 99% sequence identity) to SEQ ID NO:3.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOS:4, 5, 6, 7, 8, and 9, respectively, or an antibody comprising a heavy chain variable region comprising the amino acid sequence of SEQ ID NO:2 and a light chain variable region comprising the amino acid sequence of SEQ ID NO:3).

### Clone CL0020307

In some embodiments, an anti-TREM2 antibody comprises a CDR-H1 sequence comprising the amino acid sequence of SEQ ID NO:12, a CDR-H2 sequence comprising the amino acid sequence of SEQ ID NO:13, a CDR-H3 sequence comprising the amino acid sequence of SEQ ID NO:14, a CDR-L1 sequence comprising the amino acid sequence of SEQ ID NO:7, a CDR-L2 sequence comprising the amino acid sequence of SEQ ID NO:8, and a CDR-L3 sequence comprising the amino acid sequence of SEQ ID NO:9.

In some embodiments, an anti-TREM2 antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:10. In some embodiments, an anti-TREM2 antibody comprises a heavy chain variable region comprising the amino acid sequence of SEQ ID NO: 10.

In some embodiments, an anti-TREM2 antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:11. In some embodiments, an anti-TREM2 antibody comprises a light chain variable region comprising the amino acid sequence of SEQ ID NO:11.

In some embodiments, an anti-TREM2 antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:10 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:11. In some embodiments, an anti-TREM2 antibody comprises a heavy chain variable region comprising the amino acid sequence of SEQ ID NO:10 and a light chain variable region comprising the amino acid sequence of SEQ ID NO:11.

In some embodiments, an anti-TREM2 antibody comprises a heavy chain variable region that comprises a heavy chain CDR1-3 comprising the amino acid sequences of SEQ ID NOS:12, 13, and 14, respectively, and that has at least 85% sequence identity (e.g., at least 90%, 91%, 92%, 93%, 94%, 95%, 96%, 97%, 98%, or 99% sequence identity) to SEQ ID NO:10. In some embodiments, an anti-TREM2 antibody comprises a light chain variable region that comprises a light chain CDR1-3 comprising the amino acid sequences of SEQ ID NOS:7, 8, and 9, respectively, and that has at least 85% sequence identity (e.g., at least 90%, 91%, 92%, 93%, 94%, 95%, 96%, 97%, 98%, or 99% sequence identity) to SEQ ID NO:11.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOS:12, 13, 14, 7, 8, and 9, respectively, or an antibody comprising a heavy chain variable region comprising the amino acid sequence of SEQ ID NO:10 and a light chain variable region comprising the amino acid sequence of SEQ ID NO: 11).

### Binding Characteristics of Anti-TREM2 Antibodies

In some embodiments, an antibody as described herein that specifically binds to a TREM2 protein binds to TREM2 that is expressed on a cell (e.g., a primary cell or cell line that endogenously expresses TREM2, such as human macrophages, or a primary cell or cell line that has been engineered to express TREM2, e.g., as described in the Examples section below). In some embodiments, an antibody that specifically binds to a TREM2 protein as described herein binds to purified or recombinant TREM2 protein of a portion thereof, or to a chimeric protein comprising TREM2 or a portion thereof (e.g., an Fc-fusion protein comprising TREM2 or an Fc-fusion protein comprising the ecto-domain of TREM2).

In some embodiments, an antibody that specifically binds to human TREM2 protein exhibits cross-reactivity with one or more other TREM2 proteins of another species. In some embodiments, an antibody that specifically binds to human TREM2 protein exhibits cross-reactivity with a cynomolgus monkey (“cyno”) TREM2 protein. In some embodiments, an antibody that specifically binds to human TREM2 protein exhibits cross-reactivity with a mouse TREM2 protein. In some embodiments, an anti-TREM2 antibody exhibits cross-reactivity with human TREM2, cyno TREM2, and mouse TREM2.

Methods for analyzing binding affinity, binding kinetics, and cross-reactivity are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet™ (FortéBio, Inc., Menlo Park, CA)), and western blot analysis. In some embodiments, ELISA is used to determine binding affinity and/or cross-reactivity. Methods for performing ELISA assays are known in the art, and are also described in the Examples section below. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity.

### Epitopes Recognized by Anti-TREM2 Antibodies

In some embodiments, an anti-TREM2 antibody recognizes an epitope of human TREM2 that is the same or substantially the same as the epitope recognized by an antibody clone as described herein. As used herein, the term “substantially the same,” as used with reference to an epitope recognized by an antibody clone as described herein, means that the anti-TREM2 antibody recognizes an epitope that is identical, within, or nearly identical to (e.g., has at least 90% sequence identity to, or has one, two, or three amino acid substitutions, e.g., conservative substitutions, relative to), or has substantial overlap with (e.g., at least 50%, 60%, 70%, 80%, 90%, or 95% overlap with) the epitope recognized by the antibody clone as described herein.

In some embodiments, an anti-TREM2 antibody recognizes an epitope of human TREM2 that is the same or substantially the same as the epitope recognized by an antibody clone selected from the group consisting of Clone CL0020306, Clone CL0020188, Clone CL0020307, and Clone CL0020123.

In some embodiments, an anti-TREM2 antibody binds to human TREM2 at an epitope within the stalk region of TREM2. In some embodiments, an anti-TREM2 antibody recognizes an epitope of human TREM2 comprising, within, or consisting of residues 129-172 or residues 131-169 of SEQ ID NO:1. In some embodiments, an anti-TREM2 antibody recognizes an epitope of human TREM2 comprising, within, or consisting of residues 129-148 of SEQ ID NO:1. In some embodiments, anti-TREM2 antibody recognizes an epitope of human TREM2 comprising, within, or consisting of amino acid residues 143-149 of SEQ ID NO:1. In some embodiments, an anti-TREM2 antibody is an agonist that activates TREM2/DAP12 signaling (e.g., by inducing phosphorylation of a kinase such as Syk) and binds to human TREM2 at an epitope within the stalk region of TREM2. In some embodiments, an anti-TREM2 antibody binds to human TREM2 at an epitope within the stalk region of TREM2 and inhibits cleavage of TREM2 by a protease (e.g., ADAM17).

In some embodiments, an anti-TREM2 antibody binds to human TREM2 at an epitope within the Ig variable (IgV) domain of TREM2. In some embodiments, an anti-TREM2 antibody is an agonist that activates TREM2/DAP12 signaling (e.g., by inducing phosphorylation of a kinase such as Syk) and binds to human TREM2 at an epitope within the IgV domain of TREM2. In some embodiments, an anti-TREM2 antibody binds to human TREM2 at an epitope comprising or consisting of one or more of the following: (i) amino acid residues 55-63 (GEKGPCQRV (SEQ ID NO:70)) of SEQ ID NO:1, (ii) amino acids 96-107 (TLRNLQPHDAGL (SEQ ID NO:71)) of SEQ ID NO:1, and (iii) amino acid residues 126-129 (VEVL (SEQ ID NO:72)) of SEQ ID NO:1.

### Functional Characteristics of Anti-TREM2 Antibodies

In some embodiments, an anti-TREM2 antibody (e.g., an antibody having one or more CDR, heavy chain variable region, and/or light chain variable region sequences as disclosed) functions in one or more TREM2 activities as disclosed herein. For example, in some embodiments an anti-TREM2 antibody is an antibody that modulates levels of sTREM2 protein (e.g., levels of sTREM2 that are shed from the cell surface into an extracellular sample), modulates recruitment or phosphorylation of a kinase that interacts with a TREM2/DAP12 signaling complex (e.g., Syk kinase), and/or modulates one or more activities downstream of the signaling complex, such as phagocytosis, cell growth, cell survival, cell differentiation, cytokine secretion, or cell migration. In some embodiments, an anti-TREM2 antibody as disclosed herein binds soluble TREM2 protein (sTREM2) in healthy human CSF or cynomolgus CSF with better potency compared to a reference antibody. In some embodiments, the reference antibody is represented by a combination of sequences selected from the group consisting of: SEQ ID NOS:73 and 74; SEQ ID NOS:75 and 76; and SEQ ID NOS:77 and 78. In some embodiments, the potency assay is carried out substantially as described in Example 11.

In some embodiments, an anti-TREM2 antibody enhances one or more TREM2 activities (e.g., those described herein) that are induced by a ligand. In some embodiments, the ligand is a lipid ligand. Examples of TREM2 lipid ligands include, but are not limited to, 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (24OHC), 25(S)hydroxycholesterol (25OHC), 27-hydroxycholesterol (27OHC), Acyl Carnitine (AC), alkylacylglycerophosphocholine (PAF), α-galactosylceramide (KRN7000), Bis(monoacylglycero)phosphate (BMP), Cardiolipin (CL), Ceramide, Ceramide-1-phosphate (C1P), Cholesteryl ester (CE), Cholesterol phosphate (CP), Diacylglycerol 34:1 (DG 34:1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (SalP), Sphingomyelin (SM), Sphingosine, Sphingosine-1-phosphate (SolP), and Sulfatide.

Modulation of sTREM2 Shedding

In some embodiments, an anti-TREM2 antibody alters levels of sTREM2 protein in a sample, e.g., levels of sTREM2 that are shed from the cell surface into an extracellular sample. In some embodiments, an anti-TREM2 antibody decreases levels of sTREM2.

In some embodiments, an anti-TREM2 antibody decreases levels of sTREM2 if the amount of sTREM2 in a treated sample is decreased by at least 10%, at least 20%, at least 30%, at least 40%, at least 50%, at least 60%, at least 70%, at least 80%, at least 90% or more as compared to a control value. In some embodiments, an anti-TREM2 antibody decreases levels of sTREM2 if the amount of sTREM2 in a treated sample is decreased by at least 2-fold, 3-fold, 4-fold, 5-fold, 6-fold, 7-fold, 8-fold, 9-fold, 10-fold or more as compared to a control value. In some embodiments, the control value is the amount of sTREM2 in an untreated sample (e.g., a supernatant from a TREM2-expressing cell that has not been treated with an anti-TREM2 antibody, or a sample from a subject that has not been treated with an anti-TREM2 antibody) or a sample treated with an appropriate non-TREM2-binding antibody.

In some embodiments, sTREM2 shedding is measured using a sample that comprises a fluid, e.g., blood, plasma, serum, urine, or cerebrospinal fluid. In some embodiments, the sample comprises cerebrospinal fluid. In some embodiments, the sample comprises supernatant from cell cultures (e.g., supernatant from a primary cell or cell line that endogenously expresses TREM2, such as human macrophages, or a primary cell or cell line that has been engineered to express TREM2, e.g., as described in the Examples section below).

In some embodiments, the level of sTREM2 in a sample is measured using an immunoassay. Immunoassays are known in the art and include, but are not limited to, enzyme immunoassays (EIA) such as enzyme multiplied immunoassay (EMIA), enzyme-linked immunosorbent assay (ELISA), microparticle enzyme immunoassay (MEIA), immunohistochemistry (IHC), immunocytochemistry, capillary electrophoresis immunoassays (CEIA), radioimmunoassays (RIA), immunofluorescence, chemiluminescence immunoassays (CL), and electrochemiluminescence immunoassays (ECL). In some embodiments, sTREM2 levels are measuring using an ELISA assay. In some embodiments, sTREM2 levels are measured using an ELISA assay as described in the Examples section below.

### Modulation of Kinase Recruitment or Phosphorylation

In some embodiments, an anti-TREM2 antibody induces phosphorylation of a kinase that interacts with the TREM2/DAP12 signaling complex (such as, but not limited to, Syk, ZAP70, PI3K, Erk, AKT, or GSK3b). In some embodiments, an anti-TREM2 antibody induces phosphorylation of a kinase that interacts with the TREM2/DAP12 signaling complex without blocking binding of a native TREM2 ligand. In some embodiments, an anti-TREM2 antibody enhances phosphorylation of a kinase that interacts with the TREM2/DAP12 signaling complex that is induced by a TREM2 ligand (e.g., a lipid ligand). In some embodiments, an anti-TREM2 antibody induces or enhances phosphorylation of Syk. In some embodiments, an anti-TREM2 antibody induces or enhances phosphorylation of Syk if the level of Syk phosphorylation in a sample treated with the anti-TREM2 antibody is increased by at least 10%, at least 20%, at least 30%, at least 40%, at least 50%, at least 60%, at least 70%, at least 80%, at least 90% or more as compared to a control value. In some embodiments, an anti-TREM2 antibody induces phosphorylation of Syk if the level of Syk phosphorylation in a sample treated with the anti-TREM2 antibody is increased by at least 2-fold, 3-fold, 4-fold, 5-fold, 6-fold, 7-fold, 8-fold, 9-fold, 10-fold, or more as compared to a control value. In some embodiments, the control value is the level of Syk phosphorylation in an untreated sample (e.g., a sample comprising a TREM2-expressing cell that has not been treated with an anti-TREM2 antibody, or a sample from a subject that has not been treated with an anti-TREM2 antibody), or a sample that has been treated with a TREM2 ligand but not an anti-TREM2 antibody, or a sample treated with an appropriate non-TREM2-binding antibody.

For detecting and/or quantifying phosphorylation (e.g., Syk phosphorylation) in a sample, in some embodiments, an immunoassay is used. In some embodiments, the immunoassay is an enzyme immunoassay (EIA), enzyme multiplied immunoassay (EMIA), enzyme-linked immunosorbent assay (ELISA), microparticle enzyme immunoassay (MEIA), immunohistochemistry (IHC), immunocytochemistry, capillary electrophoresis immunoassay (CEIA), radioimmunoassay (RIA), immunofluorescence, chemiluminescence immunoassay (CL), or electrochemiluminescence immunoassay (ECL). In some embodiments, phosphorylation is detected and/or quantified using an immunoassay that utilizes an amplified luminescent proximity homogenous assay (AlphaLISA®, PerkinElmer Inc.).

In some embodiments, phosphorylation is measured using a sample that comprises one or more cells, e.g., one or more TREM2-expressing cells (e.g., a primary cell or cell line that endogenously expresses TREM2, such as human macrophages or iPSC-derived microglia, or a primary cell or cell line that has been engineered to express TREM2, e.g., as described in the Examples section below). In some embodiments, the sample comprises a fluid, e.g., blood, plasma, serum, urine, or cerebrospinal fluid. In some embodiments, the sample comprises tissue (e.g., lung, brain, kidney, spleen, nervous tissue, or skeletal muscle) or cells from such tissue. In some embodiments, the sample comprises endogenous fluid, tissue, or cells (e.g., from a human or non-human subject).

### Modulation of Phagocytosis

In some embodiments, an anti-TREM2 antibody enhances phagocytosis of dead cell debris, tissue debris, amyloid beta particles, or foreign material. In some embodiments, an anti-TREM2 antibody enhances phagocytosis without blocking binding of a native TREM2 ligand. In some embodiments, an anti-TREM2 antibody enhances phagocytosis that is induced by a TREM2 ligand (e.g., a lipid ligand). In some embodiments, an anti-TREM2 antibody enhances phagocytosis if the level of phagocytosis in a sample treated with the anti-TREM2 antibody is increased by at least 10%, at least 20%, at least 30%, at least 40%, at least 50%, at least 60%, at least 70%, at least 80%, at least 90% or more as compared to a control value. In some embodiments, an anti-TREM2 antibody enhances phagocytosis if the level of phagocytosis in a sample treated with the anti-TREM2 antibody is increased by at least 2-fold, 3-fold, 4-fold, 5-fold, 6-fold, 7-fold, 8-fold, 9-fold, 10-fold, or more as compared to a control value. In some embodiments, the control value is the level of phagocytosis in an untreated sample, a sample that has been treated with a TREM2 ligand but not an anti-TREM2 antibody, or a sample treated with an appropriate non-TREM2-binding antibody.

In some embodiments, phagocytosis is measured using a phagocytosis assay with a labeled substrate. Phagocytosis assays are known in the art. In some embodiments, the phagocytosis assay is performed on a sample comprising cells that endogenously express TREM2, such as human macrophages or microglia. In some embodiments, the phagocytosis assay is performed on a sample comprising cells that have been engineered to express TREM2. In some embodiments, phagocytosis is measured using a human macrophage phagocytosis assay as described in the Examples section below.

### Modulation of Cell Differentiation, Function, Migration, and Survival

In some embodiments, an anti-TREM2 antibody enhances cell migration, cell survival, cell function, or cell differentiation (e.g., for myeloid cells, macrophages, and microglia, including iPSC-derived microglia and disease-associated microglia). Disease-associated microglia and methods of detecting disease-associated microglia are described in Keren-Shaul et al., Cell, 2017, 169:1276-1290. In some embodiments, an anti-TREM2 antibody enhances cell migration of one or more cell types (e.g., myeloid cells, macrophages, or microglia). In some embodiments, an anti-TREM2 antibody enhances cell survival of one or more cell types (e.g., myeloid cells, macrophages, or microglia). In some embodiments, an anti-TREM2 antibody enhances cell function of one or more cell types (e.g., myeloid cells, macrophages, or microglia). In some embodiments, an anti-TREM2 antibody enhances cell differentiation of one or more cell types (e.g., myeloid cells, macrophages, or microglia). In some embodiments, an anti-TREM2 antibody enhances the migration, survival, function, and/or differentiation of myeloid cells. In some embodiments, an anti-TREM2 antibody enhances the migration, survival, function, and/or differentiation of macrophages. In some embodiments, an anti-TREM2 antibody enhances the migration, survival, function, and/or differentiation of microglia. In some embodiments, an anti-TREM2 antibody enhances microglia activation. In some embodiments, an anti-TREM2 antibody enhances the migration, survival, function, and/or differentiation of disease-associated microglia. In some embodiments, an anti-TREM2 antibody enhances cell migration, cell survival, cell function, or cell differentiation without blocking binding of a native TREM2 ligand. In some embodiments, an anti-TREM2 antibody enhances cell migration, cell survival, cell function, or cell differentiation that is induced by a TREM2 ligand (e.g., a lipid ligand).

In some embodiments, an anti-TREM2 antibody enhances cell migration, cell survival, cell function, or cell differentiation if the level of activity (e.g., migration, survival, function, or differentiation) in a sample treated with the anti-TREM2 antibody is increased by at least 10%, at least 20%, at least 30%, at least 40%, at least 50%, at least 60%, at least 70%, at least 80%, at least 90% or more as compared to a control value. In some embodiments, an anti-TREM2 antibody enhances cell migration, cell survival, cell function, or cell differentiation if the level of activity (e.g., migration, survival, function, or differentiation) in a sample treated with the anti-TREM2 antibody is increased by at least 2-fold, 3-fold, 4-fold, 5-fold, 6-fold, 7-fold, 8-fold, 9-fold, 10-fold, or more as compared to a control value. In some embodiments, the control value is the level of activity (e.g., migration, survival, function, or differentiation) in an untreated sample (e.g., a sample that has not been treated with an anti-TREM2 antibody), a sample that has been treated with a TREM2 ligand but not an anti-TREM2 antibody, or a sample treated with an appropriate non-TREM2-binding antibody.

In some embodiments, cell migration is measured using a chemotaxis assay. Chemotaxis assays are known in the art. In some embodiments, the cell migration assay (e.g., chemotaxis assay) is performed on a sample comprising cells that endogenously express TREM2, such as human macrophages. In some embodiments, the cell migration assay (e.g., chemotaxis assay) is performed on a sample comprising cells that have been engineered to express TREM2. In some embodiments, cell migration is measured using a human macrophage chemotaxis assay as described in the Examples section below.

In some embodiments, cell survival is measured using a cell viability assay. Cell viability assays are known in the art. In some embodiments, the cell survival assay (e.g., cell viability assay) is performed on a sample comprising cells that endogenously express TREM2, such as human macrophages. In some embodiments, the cell survival assay (e.g., cell viability assay) is performed on a sample comprising cells that have been engineered to express TREM2. In some embodiments, cell survival is measured using a human macrophage viability assay as described in the Examples section below.

In some embodiments, cell function is measured using a functional assay that is appropriate for that cell. For example, in some embodiments, macrophage cell function is evaluated using a phagocytosis assay, e.g., as described in the Examples section below.

In some embodiments, cell differentiation is measured by evaluating the ability of cells that endogenously express TREM2 to differentiate. For example, in some embodiments, cell differentiation is measured by evaluating the ability of macrophages to differentiate from monocytes, e.g., as described in the Examples section below.

In some embodiments, activation of microglia is measured in vivo. In some embodiments, microglia activation is measured using TSPO-PET imaging. TSPO-PET imaging methods are known in the art.

In some embodiments, an anti-TREM2 antibody enhances microglia function without increasing neuroinflammation. Levels of neuroinflammation can be determined by measuring levels of cytokines (e.g., inflammatory cytokines), such as but not limited to TNF-α, IL-10, IL-6, IL-1ra, TGFβ, IL-15, or IFN-7. In some embodiments, cytokine levels are measured using immunoassays, for example, an enzyme immunoassay (EIA), enzyme multiplied immunoassay (EMIA), enzyme-linked immunosorbent assay (ELISA), microparticle enzyme immunoassay (MEIA), immunohistochemistry (IHC), immunocytochemistry, capillary electrophoresis immunoassay (CEIA), radioimmunoassay (RIA), immunofluorescence, chemiluminescence immunoassay (CL), or electrochemiluminescence immunoassay (ECL).

### IV. Preparation of Antibodies

In some embodiments, antibodies are prepared by immunizing an animal or animals (e.g., mice, rabbits, or rats) with an antigen or a mixture of antigens for the induction of an antibody response. In some embodiments, the antigen or mixture of antigens is administered in conjugation with an adjuvant (e.g., Freund's adjuvant). After an initial immunization, one or more subsequent booster injections of the antigen or antigens may be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity. Methods of preparing antibodies are also described in the Examples section below.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Alternatively, phage or yeast display technology can be used to identify antibodies and Fab fragments that specifically bind to selected antigens. Antibodies can also be made bispecific, i.e., able to recognize two different antigens. Antibodies can also be heteroconjugates, e.g., two covalently joined antibodies, or immunotoxins.

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors. A V_{H} or V_{L} region as described herein may optionally comprise a methionine at the N-terminus.

In some embodiments, the antibody is a chimeric antibody. Methods for making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, the antibody is a humanized antibody. Generally, a non-human antibody is humanized in order to reduce its immunogenicity. Humanized antibodies typically comprise one or more variable regions (e.g., CDRs) or portions thereof that are non-human (e.g., derived from a mouse variable region sequence), and possibly some framework regions or portions thereof that are non-human, and further comprise one or more constant regions that are derived from human antibody sequences. Methods for humanizing non-human antibodies are known in the art. Transgenic mice, or other organisms such as other mammals, can be used to express humanized or human antibodies. Other methods of humanizing antibodies include, for example, variable domain resurfacing, CDR grafting, grafting specificity-determining residues (SDR), guided selection, and framework shuffling.

As an alternative to humanization, fully human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. As another example, human antibodies can be produced by hybridoma-based methods, such as by using primary human B cells for generating cell lines producing human monoclonal antibodies.

Human antibodies can also be produced using phage display or yeast display technology. In phage display, repertoires of variable heavy chain and variable light chain genes are amplified and expressed in phage display vectors. In some embodiments, the antibody library is a natural repertoire amplified from a human source. In some embodiments, the antibody library is a synthetic library made by cloning heavy chain and light chain sequences and recombining to generate a large pool of antibodies with different antigenic specificity. Phage typically display antibody fragments (e.g., Fab fragments or scFv fragments), which are then screened for binding to an antigen of interest.

In some embodiments, antibody fragments (such as a Fab, a Fab′, a F(ab′)₂, a scFv, a V_{H}, or a V_{HH}) are generated. Various techniques have been developed for the production of antibody fragments. Traditionally, these fragments were derived via proteolytic digestion of intact antibodies. However, these fragments can now be produced directly using recombinant host cells. For example, antibody fragments can be isolated from antibody phage libraries. Alternatively, Fab′-SH fragments can be directly recovered from E. coli cells and chemically coupled to form F(ab′)₂ fragments. According to another approach, F(ab′)₂ fragments can be isolated directly from recombinant host cell culture. Other techniques for the production of antibody fragments will be apparent to those skilled in the art.

In some embodiments, an antibody or an antibody fragment is conjugated to another molecule, e.g., polyethylene glycol (PEGylation) or serum albumin, to provide an extended half-life in vivo.

In some embodiments, multispecific antibodies comprising an anti-TREM2 antibody (or antigen-binding fragment thereof) as described herein are provided, e.g., a bispecific antibody. Multispecific antibodies are antibodies that have binding specificities for at least two different sites. In some embodiments, a multispecific antibody (e.g., a bispecific antibody) has a binding specificity for TREM2 and has a binding specificity for at least one other antigen. In some embodiments, a multispecific antibody (e.g., a bispecific antibody) binds to two different TREM2 epitopes. In some embodiments, a multispecific antibody (e.g., a bispecific antibody) is capable of inducing TREM2 clustering at the cell surface. An illustrative method for measuring receptor clustering using confocal FRET microscopy is described in Wallrabe et al., Biophys. J., 2003, 85:559-571. Methods of making multispecific antibodies (e.g., bispecific antibodies) include, but are not limited to, recombinant co-expression of two pairs of heavy chain and light chain in a host cell, “knobs-into-holes” engineering, intramolecular trimerization, and fusion of an antibody fragment to the N-terminus or C-terminus of another antibody, e.g., tandem variable domains.

### V. Nucleic Acids, Vectors, and Host Cells

In some embodiments, the anti-TREM2 antibodies as disclosed herein are prepared using recombinant methods. Accordingly, in some aspects, the disclosure provides isolated nucleic acids comprising a nucleic acid sequence encoding any of the anti-TREM2 antibodies as described herein (e.g., any one or more of the CDRs, heavy chain variable regions, and light chain variable regions described herein); vectors comprising such nucleic acids; and host cells into which the nucleic acids are introduced that are used to replicate the antibody-encoding nucleic acids and/or to express the antibodies.

In some embodiments, a polynucleotide (e.g., an isolated polynucleotide) comprises a nucleotide sequence encoding an antibody or antigen-binding portion thereof as described herein (e.g., as described in the Section above entitled “Anti-TREM2 Antibody Sequences”). In some embodiments, the polynucleotide comprises a nucleotide sequence encoding one or more amino acid sequences (e.g., CDR, heavy chain, or light chain sequences) disclosed in the Informal Sequence Listing below. In some embodiments, the polynucleotide comprises a nucleotide sequence encoding an amino acid sequence having at least 85% sequence identity (e.g., at least 85%, at least 90%, at least 91%, at least 92%, at least 93%, at least 94%, at least 95%, at least 96%, at least 97%, at least 98%, or at least 99% sequence identity) to a sequence (e.g., a CDR, heavy chain, or light chain sequence) disclosed in the Informal Sequence Listing below. In some embodiments, a polynucleotide as described herein is operably linked to a heterologous nucleic acid, e.g., a heterologous promoter.

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, ml18, ml19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

Suitable host cells for cloning or expressing a polynucleotide or vector as described herein include prokaryotic or eukaryotic cells. In some embodiments, the host cell is prokaryotic. In some embodiments, the host cell is eukaryotic, e.g., Chinese Hamster Ovary (CHO) cells or lymphoid cells. In some embodiments, the host cell is a human cell, e.g., a Human Embryonic Kidney (HEK) cell.

In another aspect, methods of making an anti-TREM2 antibody as described herein are provided. In some embodiments, the method includes culturing a host cell as described herein (e.g., a host cell expressing a polynucleotide or vector as described herein) under conditions suitable for expression of the antibody. In some embodiments, the antibody is subsequently recovered from the host cell (or host cell culture medium).

### VI. Therapeutic Methods Using Anti-TREM2 Antibodies

In another aspect, therapeutic methods using an anti-TREM2 antibody as disclosed herein (e.g., an anti-TREM2 antibody as described in Section III above) are provided. In some embodiments, methods of treating a neurodegenerative disease are provided. In some embodiments, methods of modulating one or more TREM2 activities (e.g., in a subject having a neurodegenerative disease) are provided.

In some embodiments, methods of treating a neurodegenerative disease are provided. In some embodiments, the neurodegenerative disease is selected from the group consisting of Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, and tangle only dementia. In some embodiments, the neurodegenerative disease is Alzheimer's disease. In some embodiments, the neurodegenerative disease is Nasu-Hakola disease. In some embodiments, the neurodegenerative disease is frontotemporal dementia. In some embodiments, the neurodegenerative disease is Parkinson's disease. In some embodiments, the method comprises administering to the subject an isolated antibody or an antigen-binding fragment thereof that specifically binds to a human TREM2 protein, e.g., an anti-TREM2 antibody as described herein, or a pharmaceutical composition comprising an anti-TREM2 antibody as described herein.

In some embodiments, an anti-TREM2 antibody (or antigen-binding portion or pharmaceutical composition thereof) as described herein is used in treating a neurodegenerative disease that is characterized by a mutation in TREM2. In some embodiments, the neurodegenerative disease that is characterized by a mutation in TREM2 is Alzheimer's disease, e.g., Alzheimer's disease that is characterized by a R47H mutation in TREM2.

In some embodiments, methods of modulating one or more TREM2 activities in a subject (e.g., a subject having a neurodegenerative disease) are provided. In some embodiments, the method comprises modulating levels of sTREM2; modulating recruitment or phosphorylation of a kinase that interacts with a TREM2/DAP12 signaling complex (e.g., Syk kinase); modulating phagocytosis (e.g., phagocytosis of cell debris, amyloid beta particles, etc.); modulating cell migration (e.g., migration of myeloid cells, macrophages, microglia, and disease associated microglia); and/or modulating cell differentiation (e.g., for myeloid cells, macrophages, microglia, and disease associated microglia). In some embodiments, methods of enhancing one or more TREM2 activities in a subject having a neurodegenerative disease are provided. In some embodiments, methods of decreasing levels of sTREM2 in a subject having a neurodegenerative disease are provided. In some embodiments, the method of modulating one or more TREM2 activities in a subject comprises administering to the subject an isolated antibody or an antigen-binding portion thereof that specifically binds to a human TREM2 protein, e.g., an anti-TREM2 antibody as describe herein, or a pharmaceutical composition comprising an anti-TREM2 antibody as described herein.

In some embodiments, the subject to be treated is a human, e.g., a human adult or a human child.

In some embodiments, methods of reducing plaque accumulation in a subject having a neurodegenerative disease are provided. In some embodiments, the method comprises administering to the subject an antibody or pharmaceutical composition as described herein. In some embodiments, the subject has Alzheimer's disease. In some embodiments, the subject is an animal model of a neurodegenerative disease (e.g., a 5XFAD or APP/PS1 mouse model). In some embodiments, plaque accumulation is measured by amyloid plaque imaging and/or Tau imaging, e.g., using positron emission tomography (PET) scanning. In some embodiments, administration of an anti-TREM2 antibody reduces plaque accumulation by at least 20%, at least 30%, at least 40%, at least 50%, at least 60%, at least 70%, at least 80%, or at least 90% as compared to a baseline value (e.g., the level of plaque accumulation in the subject prior to administration of the anti-TREM2 antibody).

In some embodiments, an anti-TREM2 antibody is administered to a subject at a therapeutically effective amount or dose. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

The route of administration of an anti-TREM2 antibody as described herein can be oral, intraperitoneal, transdermal, subcutaneous, intravenous, intramuscular, intrathecal, inhalational, topical, intralesional, rectal, intrabronchial, nasal, transmucosal, intestinal, ocular or otic delivery, or any other methods known in the art. In some embodiments, the antibody is administered orally, intravenously, or intraperitoneally.

In some embodiments, the anti-TREM2 antibody (and optionally another therapeutic agent) is administered to the subject over an extended period of time, e.g., for at least 30, 40, 50, 60, 70, 80, 90, 100, 150, 200, 250, 300, 350 days or longer.

### VII. Pharmaceutical Compositions and Kits

In another aspect, pharmaceutical compositions and kits comprising an antibody that specifically binds to a human TREM2 protein are provided. In some embodiments, the pharmaceutical compositions and kits are for use in treating a neurodegenerative disease. In some embodiments, the pharmaceutical compositions and kits are for use in modulating (e.g., enhancing or inhibiting) one or more TREM2 activities, e.g., Syk phosphorylation. In some embodiments, the pharmaceutical compositions and kits are for use in modulating (e.g., decreasing) sTREM2 levels.

### Pharmaceutical Compositions

In some embodiments, pharmaceutical compositions comprising an anti-TREM2 antibody or an antigen-binding fragment thereof are provided. In some embodiments, the anti-TREM2 antibody is an antibody as described in Section III above or an antigen-binding fragment thereof.

In some embodiments, a pharmaceutical composition comprises an anti-TREM2 antibody as described herein and further comprises one or more pharmaceutically acceptable carriers and/or excipients. A pharmaceutically acceptable carrier includes any solvents, dispersion media, or coatings that are physiologically compatible and that does not interfere with or otherwise inhibit the activity of the active agent. Various pharmaceutically acceptable excipients are well-known in the art.

In some embodiments, the carrier is suitable for intravenous, intramuscular, oral, intraperitoneal, intrathecal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known in the art.

The pharmaceutical compositions described herein can be manufactured in a manner that is known to those of skill in the art, e.g., by means of conventional mixing, dissolving, granulating, dragee-making, emulsifying, encapsulating, entrapping or lyophilizing processes. The following methods and excipients are merely exemplary and are in no way limiting.

For oral administration, an anti-TREM2 antibody can be formulated by combining it with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

An anti-TREM2 antibody can be formulated for parenteral administration by injection, e.g., by bolus injection or continuous infusion. For injection, the compound or compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, compounds can be formulated in aqueous solutions, e.g., in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

In some embodiments, an anti-TREM2 antibody is prepared for delivery in a sustained-release, controlled release, extended-release, timed-release or delayed-release formulation, for example, in semi-permeable matrices of solid hydrophobic polymers containing the active agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients. Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions of the disclosure may vary depending on the particular use envisioned. The determination of the appropriate dosage or route of administration is well within the skill of one in the art. Suitable dosages are also described in Section VI above.

### Kits

In some embodiments, kits comprising an anti-TREM2 antibody or an antigen-binding fragment thereof are provided. In some embodiments, the anti-TREM2 antibody is an antibody as described in Section III above or an antigen-binding fragment thereof.

In some embodiments, the kit further comprises one or more additional therapeutic agents. For example, in some embodiments, the kit comprises an anti-TREM2 antibody as described herein and further comprises one or more additional therapeutic agents for use in the treatment of a neurodegenerative disease, e.g., Alzheimer's disease. In some embodiments, the therapeutic agent is an agent for use in treating a cognitive or behavioral symptom of a neurodegenerative disease (e.g., an antidepressant, a dopamine agonist, or an anti-psychotic). In some embodiments, the therapeutic agent is a neuroprotective agent (e.g., carbidopa/levodopa, an anticholinergic agent, a dopaminergic agent, a monoamine oxidase B (MAO-B) inhibitor, a catechol-O-methyl transferase (COMT) inhibitor, a glutamatergic agent, a histone deacetylase (HDAC) inhibitor, a cannabinoid, a caspase inhibitor, melatonin, an anti-inflammatory agent, a hormone (e.g., estrogen or progesterone), or a vitamin).

In some embodiments, the kit comprises an anti-TREM antibody as described herein and further comprises one or more reagents for measuring sTREM2 levels. In some embodiments, the kit comprises an anti-TREM antibody as described herein and further comprises one or more reagents for measuring TREM2 activity (e.g., for measuring Syk phosphorylation).

In some embodiments, the kit further comprises instructional materials containing directions (i.e., protocols) for the practice of the methods described herein (e.g., instructions for using the kit for a therapeutic method as described in Section VI above). While the instructional materials typically comprise written or printed materials, they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD-ROM), and the like. Such media may include addresses to internet sites that provide such instructional materials.

### VIII. Examples

The present invention will be described in greater detail by way of specific examples. The following examples are offered for illustrative purposes only, and are not intended to limit the invention in any manner.

### Example 1. Generation and Initial Characterization of Anti-TREM2 Antibodies

### Recombinant Expression and Purification of Mouse Fc Fused Human TREM2 ECD

The ecto domain (residues 19-172) of human TREM2 (UniProtKB ID—Q9NZC2) was subcloned into pRK vector with the secretion signal from mouse IgG kappa chain V-III, amino acids 1-20 (UniProtKB ID—P01661) at the N-terminal region, and a mouse Fc tag at the C-terminal region with a GGGGS (SEQ ID NO:64) between TREM2 ECD and Fc.

Purified plasmid was transfected into Expi293F™ cells (Thermo Fisher) using the Expi293F™ Expression System Kit according to the manufacturer's instructions. To inhibit maturation of N-linked glycans and reduce glycosylation heterogeneity, kifunensine (Sigma), an inhibitor of high mannosidase I was added to the culture at 1 g/mL concentration immediately after transfection. Transfected cells were incubated in an orbital shaker (Infors HT Multitron) at 125 rpm and 37 °C in a humidified atmosphere of 6% CO₂. ExpiFectamine™ 293 Transfection Enhancer 1 and 2 were added to the cells 16 hours post transfection and the media supernatant was harvested 96 hours post transfection. The clarified supernatant was supplemented with EDTA-free protease inhibitor (Roche) and was stored at −80 °C.

For rhTREM2-Fc isolation, clarified media supernatant was loaded on HiTrap MabSelect SuRe Protein A affinity column (GE Healthcare Life Sciences) and washed with 200 mM arginine and 137 mM succinate buffer pH 5.0. The fusion protein was eluted in 100 mM QB citrate buffer pH 3.0 and 50 mM NaCl. Immediately after elution, 1M Tris-HCl buffer pH 8.0 was added to the protein solution to neutralize the pH. Protein aggregates were separated by size exclusion chromatography (SEC) on Superdex 200 increase 10/300 GL column (GE Healthcare Life Sciences). The SEC mobile phase buffer was kept at 20 mM Tris-HCl pH 8.0, 100 mM NaCl and 50 mM arginine, which was also the protein storage buffer. All chromatography steps were performed on AKTA pure or AKTA Avant systems (GE Healthcare Life Sciences).

### Recombinant Expression and Purification of His-Tagged TREM2 ECD

The ecto domain (residues 19-172) of TREM2 (UniProtKB—Q9NZC2) was subcloned in the pRK vector with the secretion signal from mouse Ig kappa chain V-III, amino acids 1-20 (UniProtKB ID—P01661) at the N-terminal region, and a 6×-His tag (SEQ ID NO:65) at the C-terminal region. The insert was verified by sequencing and maxi prep plasmid purification was performed.

Purified plasmid was transfected into Expi293F™ cells (Thermo Fisher) using the Expi293F™ Expression System Kit according to the manufacturer's instructions. Transfected cells were incubated in an orbital shaker (Infors HT Multitron) at 125 rpm and 37 °C in a humidified atmosphere of 6% CO₂. ExpiFectamine™ 293 Transfection Enhancer 1 and 2 were added to the cells 16 hours post transfection and the media supernatant was harvested 96 hours post transfection.

Harvested media was supplemented with 1M imidazole pH 8.0 to a final concentration of 10 mM and filtered using the Nalgene™ Rapid-Flow™ disposable filter units (Thermo Fisher) with a pore size of 0.4 microns. HisPur™ Ni-NTA Resin (Thermo Fisher) was washed with MQ water and equilibrated with load buffer (20 mM Tris pH 8.0, 150 mM NaCl, and 10 mM imidazole). Affinity purification was performed using the gravity flow method. The harvested media was loaded onto the resin and nonspecifically bound proteins were washed with load buffer supplemented with 50 mM and 100 mM imidazole. The bound His-tagged TREM2 eco domain was eluted with 20 mM Tris pH 8.0, 150 mM NaCl, and 200 mM imidazole. Eluted protein was concentrated using Amicon 10 kDa concentrators and the concentrated protein was further purified by gel filtration chromatography using the AKTA Avant system (GE Healthcare Life Sciences). The protein was loaded onto a HiLoad Superdex 200 16/600 (GE Healthcare Life Sciences) column equilibrated with 1×PBS and eluted and fractionated using 1×PBS as the running buffer. Eluted fractions were analyzed by electrophoresis on polyacrylamide (PAGE) gels under denaturing and native conditions. Eluted fractions were further characterized by analytical size exclusion chromatography and the intact protein mass determination. Results from the PAGE and analytical characterization were used to pool the heavily glycosylated protein fractions and these were aliquoted and stored at −80 °C.

### Generation of Antibodies

Rodents (mice and rats) were immunized using standard protocols with rhTREM2-Fc immunogen or BWZ cells expressing full length Trem2 receptor. Titers were measured throughout immunization using sera collected at different time points. The detection of an antigen specific immune response was performed using flow cytometry with the rhTREM2-Fc immunogen and live BWZ cells expressing full-length TREM2. Selection criteria of candidate antibodies included rodent antibody production and specificity of binding to TREM2 as detected by flow cytometry. Antibody-secreting cells were isolated from animal immune tissues including spleen, lymph nodes and bone marrow.

Single cell suspensions were analyzed to determine the binding properties of secreted antibodies. Antibody-secreting cells were loaded into microfluidic devices and isolated in nanoliter volume reaction chambers to enable the detection of secreted antibodies using fluorescent and brightfield image-based microscopy assays (see, e.g., U.S. Pat. No. 9,188,593). Binding assays involving detection of antibodies binding to antigen-coated micro-beads, detection of soluble fluorescently-labeled antigen binding to antibodies immobilized on beads, and detection of antibody binding to cell surface-expressed antigens were carried out. Cell surface-expressed antigens included both recombinant form and the native forms of antigens presented on the surface of cells.

Image analysis was used to identify chambers exhibiting positive fluorescent signals, indicating the presence of a single cell producing antibodies with the desired properties, and the contents of chambers were recovered and lysed in 384 well plates (see, e.g., U.S. Pat. No. 10,087,408). Single cell lysates were then subjected to RT-PCR to amplify the heavy and light chain variable region sequences. The resulting amplicons were then sequenced to determine the cDNA sequence of paired heavy and light chain variable regions from the selected single cells. The resulting sequences were manually inspected and analyzed to determine sequence diversity and somatic hypermutation. Sequences were selected for expression based on screening data and sequence diversity. Expressed antibodies were tested to confirm antigen binding specificity.

### Primary Screening of Anti-TREM2 Antibodies

Primary screening of antibodies was performed in HEK 293 cells expressing TREM2, wild-type iPSC, and TREM2 knockout iPSC as follows.

### 1. Screening for TREM2 Binding in TREM2-Expressing HEK Cells

A HEK 293 cell line stably expressing human TREM2/DAP12 was generated by transfecting the cells with a vector expressing wild type human TREM2 and DAP12, and DAP12 alone, respectively. Stable expressing clones were selected, and the cell surface TREM2 expression was evaluated by flow cytometry. APC-conjugated rat anti-human/mouse-TREM2 monoclonal antibody (R&D, Catalog No. MAB17291) was used to detect surface TREM2 expression. The clone showing the highest wild type TREM2 expression level was selected and named “HEK293-H6.” The clones stably expressing DAP12 were analyzed by Western blot, and the selected clone was named “HEK293-DAP12 #1.”

HEK 293 overexpressing human TREM2 (HEK293-H6) and HEK 293 overexpressing GFP (B5) were harvested by 0.05% trypsin and incubated at 37 °C for 2 hours. After incubation, the cells were centrifuged and washed in FACS buffer (PBS+0.5% BSA) twice. Mixed cells were resuspended in FACS buffer with human Trustain FcX solution (Biolegend, Catalog No. 422302) at a density of 10⁶/mL per cell line. The mixed cell lines were seeded at 200,000 cells per well in a 96-well round-bottom plate and incubated for 20 minutes at room temperature. After incubation, the cells were centrifuged and incubated with anti-TREM2 antibodies of about 0-200 nM dose titration for 45 minutes on ice. After incubation, the cells were centrifuged and washed with FACS buffer three times. The cells were then incubated with secondary antibody (Alexa Fluor 647 AffiniPure F(ab′)2 Fragment Goat Anti-human IgG(H+L), Jackson ImmunoResearch Laboratories, Catalog No. 109-606-088, 1:800 dilution) for 30 minutes on ice. After incubation, the cells were washed with FACS buffer three times, resuspended in 100 μL of FACS buffer, and analyzed by flow cytometry (BD FACSCanto II, San Jose, CA), for which 30,000 events were obtained for each sample. Mean fluorescence intensity per cells were calculated by FlowJo software and used for generating dose response binding curve.

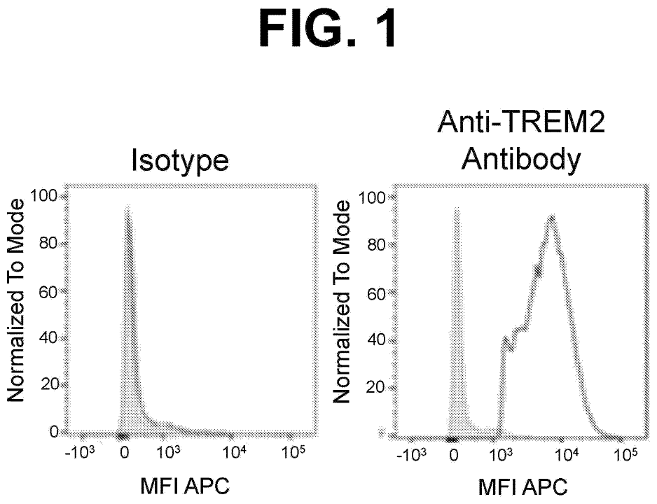

FIG. 1 illustrates a representative result for an exemplary antibody that binds cell surface receptor TREM2 in HEK293-H6 cells.

Evaluation of Activation of TREM2-Dependent pSyk Signaling

Activation of TREM2-dependent pSyk signaling was measured in human macrophage cells or in HEK293-H6 cells using a commercial AlphaLisa assay from Perkin-Elmer.

For all experiments involving use of lipid vesicles containing 70% DOPC and 30% POPS, the lipid vesicles were prepared within two weeks of experiments as follows: 7 mg DOPC (1,2-dioleoyl-sn-glycero-3-phosphocholine) and 3 mg POPS (1-palmitoyl-2-oleoyl-sn-glycero-3-phospho-L-serine) were combined in chloroform in a glass vial and dried under a stream of N₂ gas for 1-2 hours, or until completely dry. The lipid mixture was re-suspended in 1 mL HBSS (for a final lipid concentration of about 10 mg/mL) and vortexed for 2-3 minutes. Subsequently, the lipid suspension was extruded using an Avanti mini-extruder constructed with one 100-nm pore size membrane to form small unilamellar vesicles at 10 mg/mL.

### 1. Dosing of Antibodies in Cells

The day before assay, human macrophage cells or HEK293-H6 cells were plated at 100,000 cells/well or 40,000 cells/well, respectively, on a 96-well plate coated with poly-D-lysine. Antibodies were diluted into PBS starting at 300 nM and proceeding in a 10-point serial dilution titration with 3-fold dilutions between points. For antagonist dose-response curves, lipid vesicles containing 70% DOPC and 30% POPS at 1 mg/mL final concentration were also included in the antibody/PBS mixture. The cells were washed 3 times with HBSS using a Biotek 405/406 plate washer, after which 50 μL per well of the antibody/PBS (with or without vesicles) solution was added using a Hamilton Nimbus liquid handler. The cell plate was then transferred to a 37 °C incubator for 5 minutes. The liposome/antibody solution was removed by flicking the plate, and 40 μL lysis buffer (Cell Signaling Technologies, CST) containing 1 μM PMSF was added using the liquid handler. The lysate was then either frozen at −80 °C or immediately assayed in the AlphaLisa assay.

Human macrophage cells were prepared for assay as follows. Human monocytes were isolated following the RosetteSep human monocyte enrichment cocktail protocol (Stemcell Technologies, REF #15068) from fresh blood. Isolated monocytes were washed in wash buffer (PBS+2% FBS) and resuspended in 10 mL ACK lysis buffer (ThermoFisher Scientific, Catalog No. A10492) to lyse red blood cells. Twenty (20) mL of wash buffer was added to stop cell lysis, and the sample was centrifuged and washed once more with culture media (RPMI, 10% Hyclone FBS, 1% Sodium Pyruvate, 1% Glutamax, 1% non-essential amino acids, and 1% Penicillin-streptomycin). Human monocytes were then differentiated into macrophage cells in culture media in the presence of 50 ng/mL human recombinant M-CSF (Gibco, Catalog No. PHC9501) at 250-mL flask. Fresh human M-CSF was spiked on day 3 and human macrophages were subsequently harvested on day 5 and used for assay.

### 2. AlphaLisa Assay

Cell lysates were assayed for pSyk using the standard protocol for the Perkin Elmer pSyk AlphaLisa kit. In brief, 10 μL of lysate/well was transferred to a white opaque 384 well Optiplate (Perkin Elmer). Next, 5 μL of Acceptor Mix (containing the working solution of acceptor beads) was added per well, followed by sealing of plates with foil seals and incubation for 1 hour at room temperature. Subsequently, 5 μL of Donor Mix (containing the working solution of donor beads) was added to each well under reduced light conditions. Plates were again sealed and incubated for 1 hour at room temperature. Finally, the plates were read using AlphaLisa settings on a Perkin Elmer EnVision plate reader.

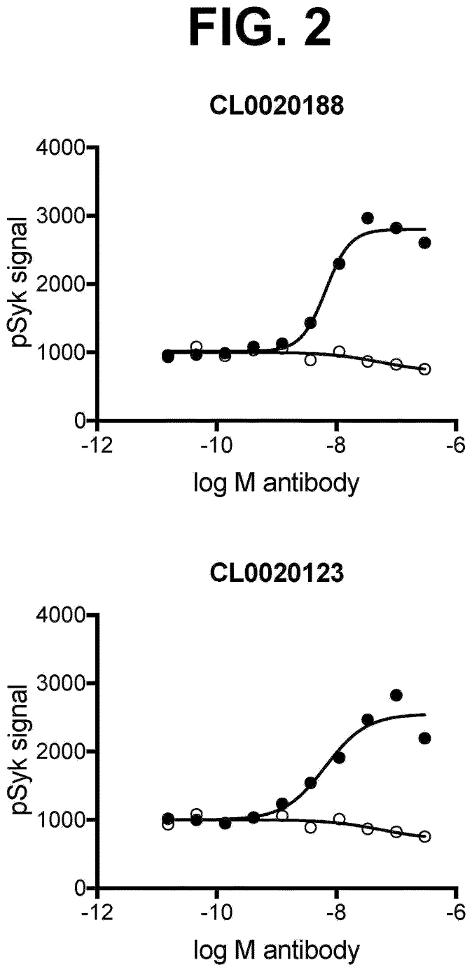

FIG. 2 illustrates representative anti-TREM2 antibody dose response curves for pSyk signal activation in primary human macrophage cells. Solid black circles (●) represent anti-TREM2 antibody, and open white circles (°) represent isotype control. Each curve represents the mean of three independent experiments, and EC₅₀ values are provided in Table 1 below. The results indicate that anti-TREM2 antibodies are able to activate TREM2-DAP12 ITAM signaling in primary human macrophages.

Liposome Response Assay in iPSC Microglia

TREM2 agonist antibodies and phosphotidylserine-containing liposomes activate pSyk via TREM2. In order to understand the effect of anti-TREM2 antibodies on Syk signaling in the presence of liposomes, iPSC microglia were pre-treated with anti-TREM2 antibody, followed by assessment of the liposome response in the cells.

Prior to assay, iPSCs were first differentiated into hematopoietic progenitor cells (HPCs) using a commercially available kit (STEMdiff Hematopoietic Kit from StemCell Technologies). HPCs were transferred to a plate containing primary human astrocytes and co-cultured for 14-21 days. Once floating cells in co-culture were predominantly identified as mature microglia (>80%), the microglia were used for assay.

Two days prior to assay, human iPSC microglia were plated at 30,000 cells/well on a 96-well plate coated with poly-D-lysine. Antibodies were diluted to 100 nM into media containing IMDM, 10% Hyclone FBS, and 1% Pen-strep, and the cells were dosed with the antibody solution for 24 hours or 5 minutes at 37 °C. Subsequently, the cells were washed once with HBSS and then dosed with lipid vesicles containing 70% DOPC and 30% POPS at 1 mg/mL for 5 minutes at 37 °C. The liposome solution was removed by flicking the plate, and L lysis buffer (Cell Signaling Technologies, CST) containing 1 μM PMSF was added. The lysate was then either frozen at −80 °C or immediately assayed in the AlphaLisa assay. The cell lysates were assayed for pSyk using the standard protocol for the Perkin Elmer pSyk AlphaLisa kit as described above.

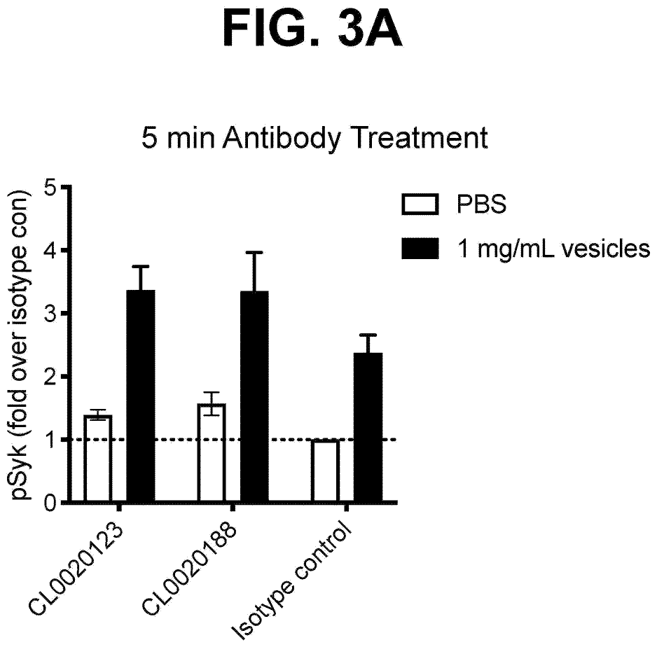

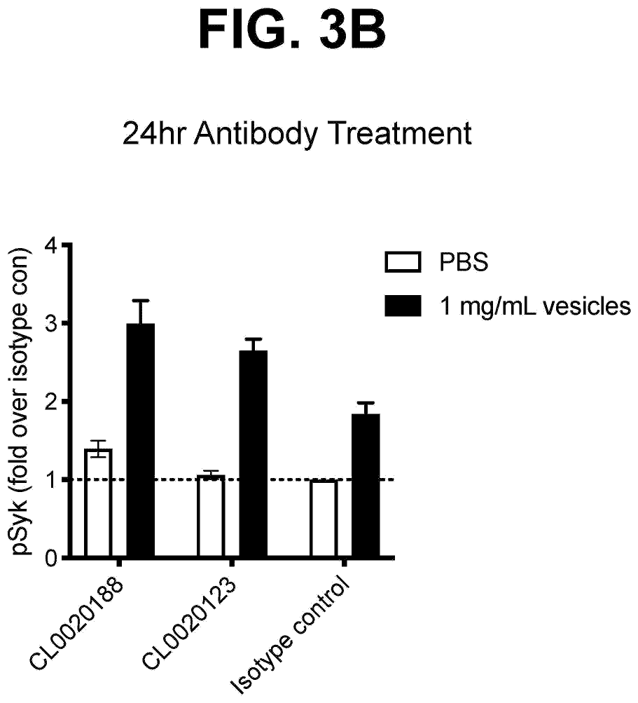

FIGS. 3A and 3B illustrate the activation of pSyk signal in human iPSC microglia incubated with anti-TREM2 antibodies, followed by dosing with lipid vesicles and assessment of the liposome response in the cells. White bars indicate incubation with PBS instead of lipid vesicles as a control. The data represent the mean and standard error of 2-7 independent experiments. FIG. 3A illustrates data for iPSC microglia pre-treated with antibody for 5 minutes, and FIG. 3B illustrates data for iPSC microglia pre-treated with antibody for 24 hours. The results show that pre-treatment of human iPSC microglia with anti-TREM2 antibodies produces an increase in the phospho-Syk signal elicited by liposomes compared to isotype control, indicating that the anti-TREM2 antibodies do not interfere with, but instead enhance, lipid activation of pSyk signaling in cells.

### Human TREM2 NFAT Reporter Assay

Human TREM2/DAP12-expressing Jurkat NFAT cell lines were generated as follows. Jurkat NFAT reporter cells were infected with lentiviral vector expression of human TREM2 and DAP12 and cultured in RPMI containing 10% Hyclone FBS and 1% penicillin/streptomycin. Stable expressing clones were selected in the presence of puromycin and Zeocin. The cell surface TREM2 expression was evaluated by flow cytometer using a biotinylated anti-TREM2 antibody (SEQ ID NOS:66 and 67). The clone that illustrated the highest wild type TREM2 expression level was selected and named as hTrem2/NFAT Jurkat reporter cells for the assay described below.

The day prior to assay, 96-well plates were pre-coated with anti-TREM2 antibody or isotype control at a 0-500 nM dose titration (45 μL/well, total 12 points) and incubated overnight at 4 C. After overnight incubation, the pre-coated plate was washed twice with PBS and then loaded with hTrem2/NFAT Jurkat reporter cells (10⁶ cells/well) in 200 μL fresh culture media (RPMI with 10% Hyclone FBS and 1% penicillin/streptomycin). The plate was incubated at 37 °C for 24 hours, after which 50 μL/well of quantlucia solution were added to each well and mixed well. For analysis, 20 μL of solution were removed from each well and transferred to a 384-well white plate for measurement of signal by luminometer (Perkin Elmer Envision).

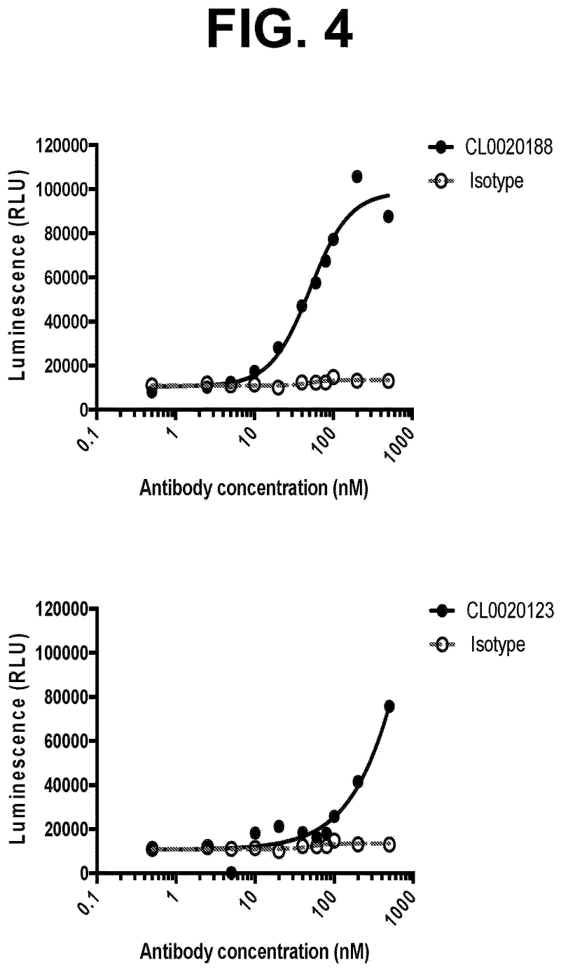

FIG. 4 includes representative anti-TREM2 antibody dose-response curves for activation of NFAT as measured by detection of the reporter gene luciferase, and EC₅₀ values for activation are provided in Table 1 below. The results in FIG. 4 illustrate that relative to isotype control, candidate anti-TREM2 antibodies were capable of inducing NFAT activation and sufficient downstream signaling to activate a transcriptional response.

### Survival Assay in Human Macrophage Cells

Human monocytes were isolated following the RosetteSep human monocyte enrichment cocktail protocol (Stemcell Technologies, Catalog No. 15068). Isolated monocytes were washed in wash buffer (PBS+2% FBS) and resuspended in 10 mL ACK lysis solution (ThermoFisher Scientific, Catalog No. A10492) to lyse red blood cells. Twenty (20) mL wash buffer was added to stop lysis. The cell suspension was centrifuged and washed once with culture media (RPMI 1640+10% FBS+penicillin/streptomycin). Cells were resuspended in culture media at a density of 10⁶ cells μL/mL and used in the survival assay described below.

The day prior to assay, 96-well plates were pre-coated with anti-TREM2 antibody or isotype control at a 0-200 nM dose titration (45 μL/well, total 12 points) and incubated overnight at 4 °C. After overnight incubation, the pre-coated plate was washed twice with PBS and then loaded with human monocyte (10⁵ cells/well) in the presence of low concentration human M-CSF (5 ng/mL, Gibco, Catalog No. PHC9501). After 5 days at 37 °C, the media was aspirated, and 100 μL PBS+100 μL Celltiter-glo media (Promega, Catalog No. G7571) was added to each well. After 10 minutes of incubation, the cell media was transferred to multiwell plates compatible for luminometer use, and luminescence for cell viability was recorded.

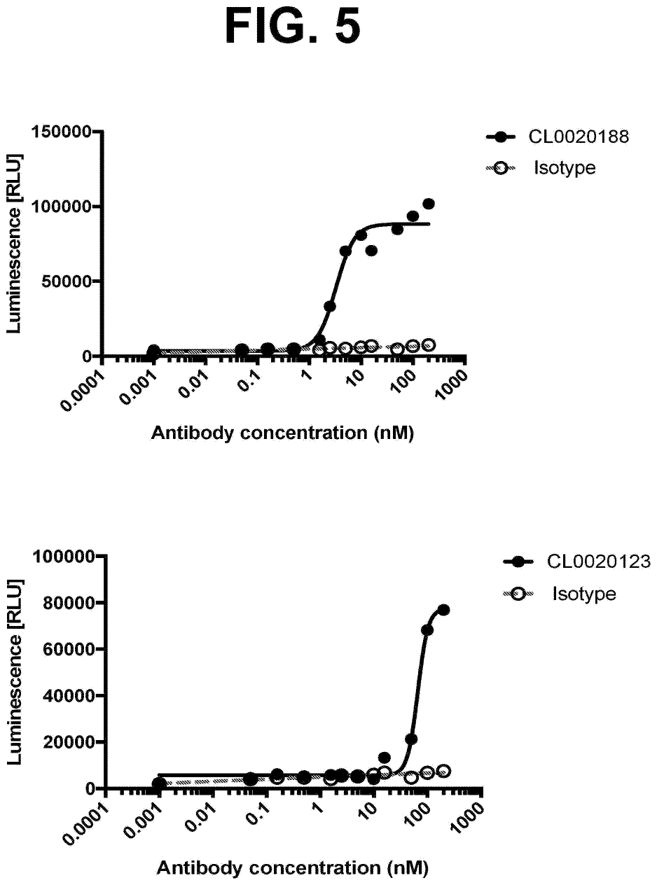

FIG. 5 illustrates representative anti-TREM2 antibody dose-response curves of cell survival in human macrophage cells under low M-CSF conditions, and EC₅₀ values for survival are provided in Table 1 below. The results indicate that TREM2 agonist antibodies have sufficient receptor activating capacity to induce a transcriptional response for modulating cellular function and promoting survival of human macrophage cells under low M-CSF conditions.

### Biacore Kinetic Measurement of Antibodies

Surface plasmon resonance (Biacore™ 8K instrument) was used to measure anti-TREM2 antibody affinities for human and cynomolgus TREM2 ECD. Anti-TREM2 antibodies were captured using Human Fab Capture Kit (GE Healthcare Life Sciences, Catalog No. 28958325) on a Biacore Series S CM5 sensor chip (GE Healthcare Life Sciences, Catalog No. 29149604). Serial 3-fold dilutions of recombinant human or cynomolgus TREM2 were injected at a flow rate of 30 μL/min. Antibody binding was monitored for 300 seconds, followed by monitoring of antibody dissociation for 600+ seconds in HBS-EP+ running buffer (GE Healthcare Life Sciences, Catalog No. BR100669). The binding response was corrected by subtracting the RU value from a blank flow cell. A 1:1 Languir model of simultaneous fitting of kₒₙ and k_{off} was used for kinetics analysis. K_{D} binding values are provided in Table 1 below.

TABLE 1
In Vitro Characteristics of Antibodies

| | | | | | | NFAT | Survival |
|---|---|---|---|---|---|---|---|
| | | | pSyk | pSyk | | EC50 | EC50 |
| | | Biacore | activation | activation | Cell binding | (nM)* | (nM)* |
| | Biacore | cyno | (human | (HEK293- | (human | *Plate | *Plate |
| | hTREM2 | TREM2 | macrophages) | H6) | macrophages) | coated | coated |
| Antibody | K_{D} (nM) | K_{D} (nM) | EC50 (nM) | EC50 (nM) | EC50 (nM) | mAb | mAb |
| CL0020123 | 0.068 | 5.7 | 7 ± 2.7 | | 0.3 | Not | 44.7 ± 30.3 |
| | | | | | | Calculable | |
| CL0020188 | 5.2 | 2.0 | 7.7 ± 1.5 | | 0.5 | 91.2 ± 47.0 | 2.4 ± 1.3 |

NB: no binding detected
ND: not determined

### Example 2. Modulation of Soluble TREM2 Levels and Phagocytosis Behavior in Human Macrophage Cells

### Soluble TREM2 Dose Response Assay in Human Macrophages

Human macrophage cells were generated as described above. One day prior to assay, human macrophage cells were plated at 100,000 cells/well on a 96 well plate coated with poly-D-lysine. Antibodies were diluted in human macrophage media (RPMI, 10% Hyclone FBS, 1% Sodium Pyruvate, 1% Glutamax, 1% non-essential amino acids, and 1% Penicillin-streptomycin) starting from 300 nM and proceeding in a 10-point serial dilution titration with 3-fold dilutions between points. The cells were dosed with the antibodies and incubated for 24 hours. After incubation with the antibodies, the plate was spun down to remove debris, and the supernatants collected for soluble TREM2 measurement.

Soluble TREM2 was measured as follows. Briefly, MSD small spot streptavidin plates (Meso Scale Discovery) were coated with biotinylated anti-hTREM2 polyclonal antibody (R&D Systems) overnight at 4 °C. The plates were then blocked with 3% BSA/TBST for 1 hour at room temperature. Samples and standards were prepared by heating to 95 °C for 5 minutes in SDS-containing buffer. The prepared samples and standards were diluted 1:10 in 3% BSA/TBST in the assay plate after blocking. A TREM2-His protein diluted in 3% BSA/TBST was used as a standard for absolute quantification. Following a two-hour incubation at room temperature, the plates were washed with TBST. The primary detection antibody, sulfo-tagged goat anti-human TREM2 (R&D Systems), was diluted in 3% BSA/TBST, added to the plates, and incubated for one hour at room temperature. After washing with TBST, the MSD plates were developed using 2×MSD read buffer T, followed by detection using an MSD Sector plate reader. MSD values were converted to absolute quantities of sTREM2 by fitting a standard curve using Prism 7.0 software (Graphpad). Modulation of TREM2 shedding was represented as a ratio of soluble TREM2 from cells incubated with test anti-TREM2 antibodies normalized to soluble TREM2 from cells cultured with no specific anti-TREM2 antibody in the media.

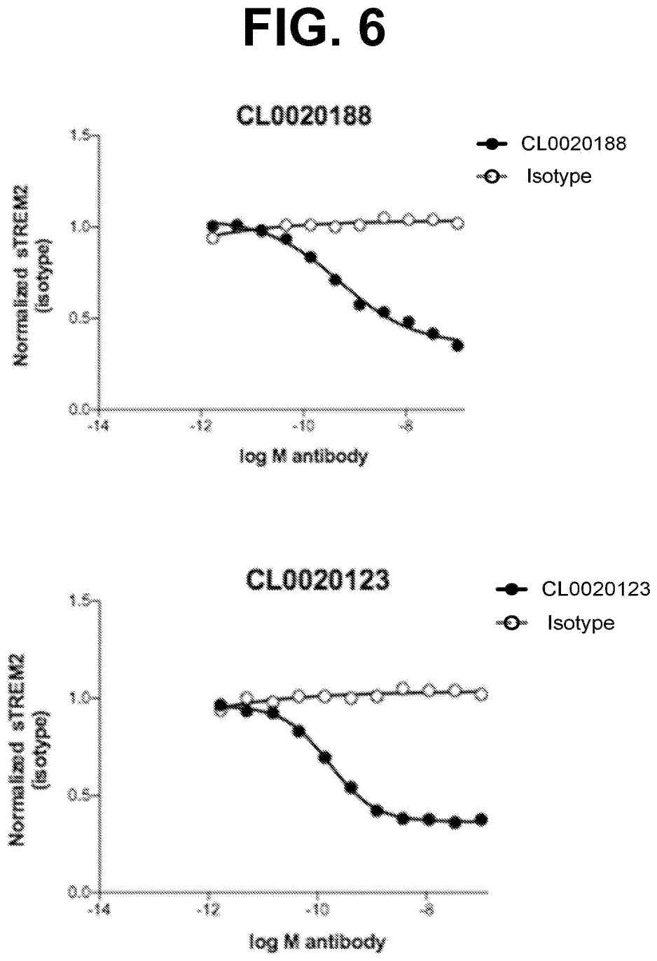

FIG. 6 illustrates representative soluble TREM2 levels (sTREM2) as a function of the anti-TREM2 antibody concentration. The results indicate the anti-TREM2 antibodies are capable of decreasing sTREM2 levels in human macrophage cells in a dose-dependent manner after overnight treatment.

### Phagocytosis Assay in Human Macrophages

Human macrophage cells were generated as described above. Two days prior to assay, human macrophage cells were plated at 80,000 cells/well on a 96 well plate coated with poly-D-lysine. Antibodies were diluted at 100 nM into media containing RPMI, 10% Hyclone FBS, 1% Sodium Pyruvate, 1% Glutamax, 1% non-essential amino acids, and 1% Penicillin-streptomycin. The cells were then dosed with antibody solution for 24 hours at 37 °C. The cell nuclei and cell membrane were then stained for 10 minutes, after which pHrodo-myelin was added at 5 μg/mL. The cells were then incubated for 4 hours at 37 °C. The pHrodo fluorescence was measured per cell on a high content confocal microscope (Opera Phoenix), and the fluorescence intensity quantified on the instrument software.

pHrodo-myelin was prepared by purifying myelin from wildtype C57Bl/6 mouse brain (Jackson Laboratories) using methods described in Safaiyan et al. (2016, Nature Neuroscience 19(8):995-998). Following purification, myelin was resuspended in PBS and adjusted to 1 mg/mL protein concentration using the DC Protein Assay Kit 2 (BioRad, Catalog No. 5000112). Myelin was tagged with pHrodo-red using a microscale labeling kit (ThermoFisher, Catalog No. P35363) according to manufacturer instructions. Excess label was removed by pelleting the myelin at 10,000 g for 5 min, removing the supernatant, and repeating these steps 3-5 times.

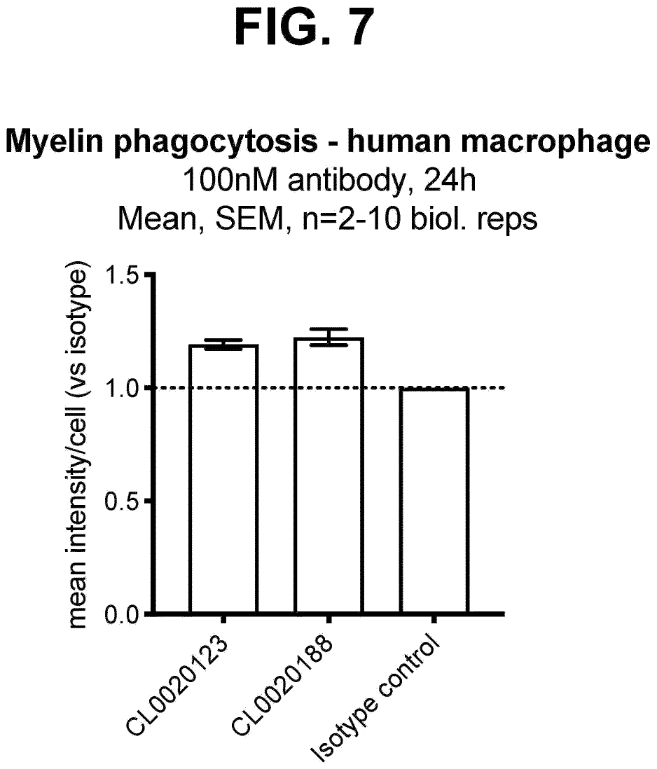

FIG. 7 illustrates representative results of the phagocytosis assay in human macrophage cells. Myelin phagocytosis was measured by detecting and quantifying pHrodo fluorescence in microscopic images of TREM2-treated macrophage cells and comparing the measured values to those of an isotype control. The results show that human macrophages treated with exemplary TREM2 agonist antibodies increase pHrodo-myelin phagocytosis relative to isotype control, indicating that the anti-TREM2 antibodies can enable beneficial clearance of myelin debris in cells.

### Example 3. Modulation of Lipid Accumulation in iPSC Microglia

### Lipid Storage Assay

Prior to assay, iPSCs were first differentiated into hematopoietic progenitor cells (HPCs) using a commercially available kit (STEMdiff Hematopoietic Kit from StemCell Technologies). HPCs were transferred to a plate containing primary human astrocytes and co-cultured for 14-21 days. Once floating cells in co-culture were predominantly identified as mature microglia (>80%), the microglia were used for assay.

Cells (iPSC microglia, 30,000 cells/well) were plated on PDL-coated 96-well plates in full serum media. After 24 hours at 37 °C, purified unlabeled myelin (50 μg/mL final concentration, purified from wildtype C57Bl/6 mouse brain (Jackson Laboratories) using methods described in Safaiyan et al. (2016, Nature Neuroscience 19(8):995-998)) was spiked into the wells. After 24 hours at 37 °C of lipid treatment, anti-TREM2 antibody or RSV control was spiked into the wells to a final concentration of 100 nM. The cells were incubated for another 48-72 hours at 37 °C before collecting or imaging cells. For myelin washout experiments, myelin was removed after the 24-hour incubation period and replaced with antibody-containing media for a subsequent 24-48 hours of incubation.

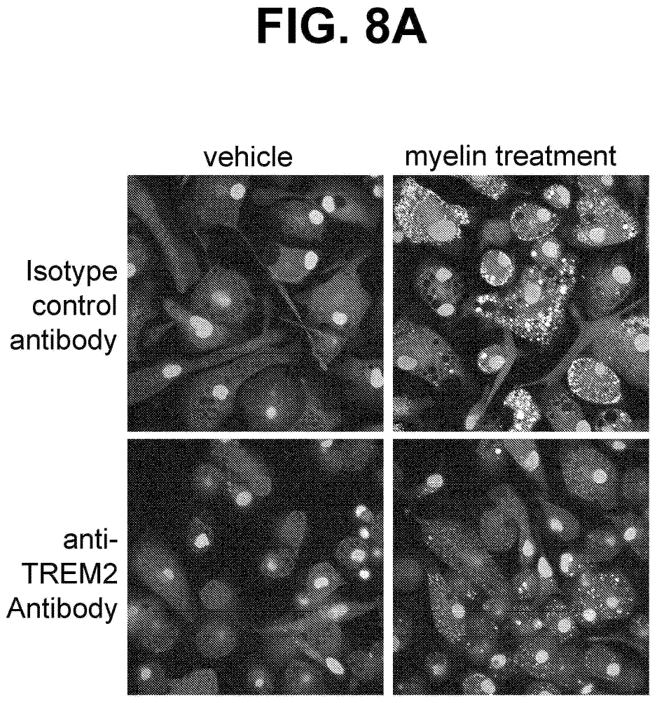

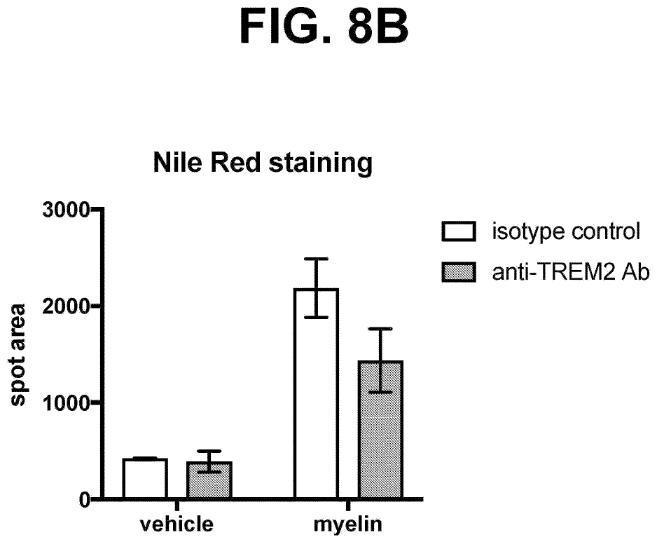

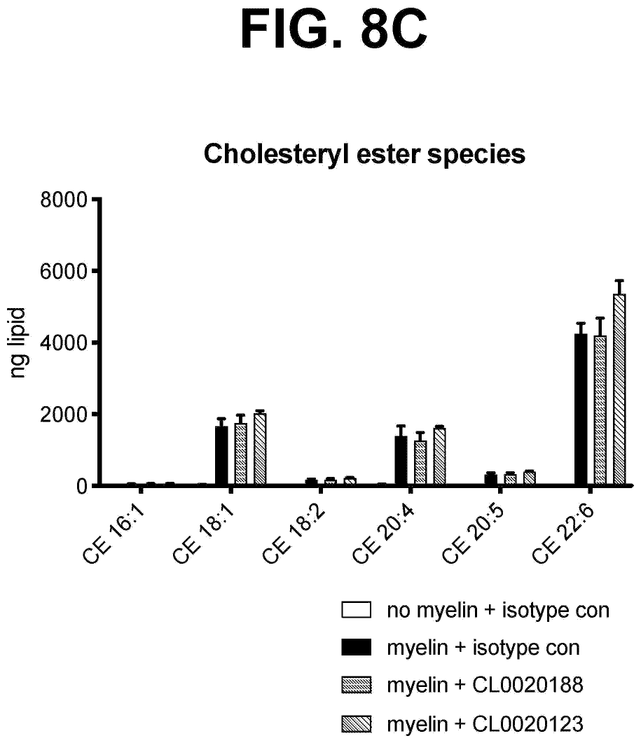

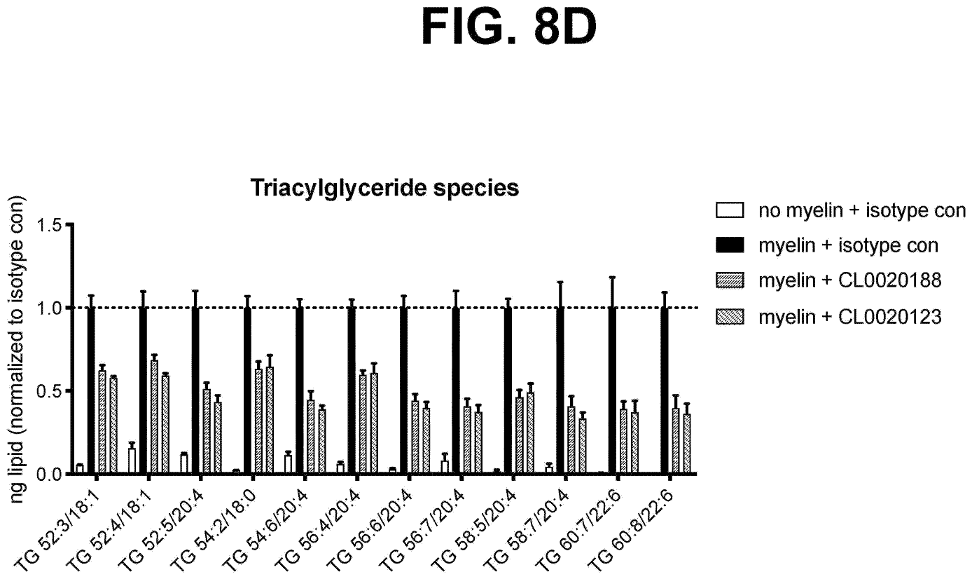

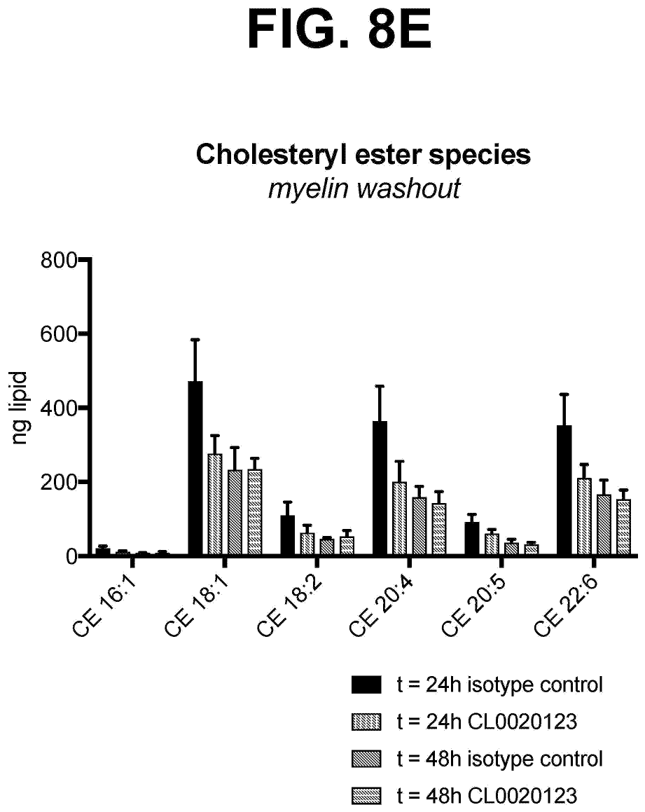

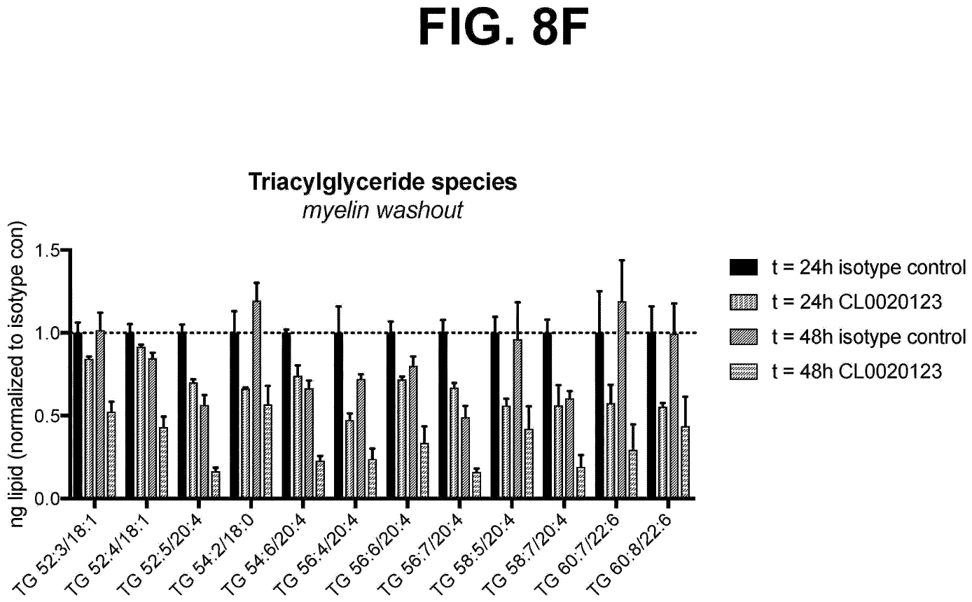

For Nile Red imaging, the supernatant was removed, and cells were incubated at 37 °C for 30 minutes in live cell imaging buffer (Life Technologies, Catalog No. A14291DJ) containing 1 μM Nile Red (ThermoFisher, Catalog No. N1142) and 1 drop/mL of Nucblue (ThermoFisher, Catalog No. R37605). After the incubation period, the staining solution was removed, and the cells were fixed in 4% paraformaldehyde. The cells were then imaged using Alexa 568 and DAPI illumination settings on an Opera Phoenix high content confocal imager. Lipid spots were analyzed using a spot-finding algorithm on the Harmony software supplied with the instrument. FIG. 8A includes a representative microscopy image of iPSC microglia treated with either vehicle or myelin (50 μg/mL final concentration) for 24 hours, followed by incubation with an isotype control or an exemplary anti-TREM2 antibody (CL0020123) for 72 hours. FIG. 8B is a representative bar chart for the same anti-TREM2 antibody used in the microscopy image of FIG. 8A. Quantification of Nile Red staining was performed by total spot intensity per cell, and data is shown as the mean and standard deviation of three technical replicates in different fields of the same microscopy sample.

For lipidomic analysis, cells were washed once with PBS while kept on ice. A volume of 70 μL of a 9:1 methanol:water solution containing 1:100 internal standards was added to the cells in the 96-well plate. The plate was agitated on a shaker at 4 °C and 1200 rpm for 20 minutes and then centrifuged for 5 minutes at 300×g. A 50 μL sample of supernatant was transferred to LCMS vials and kept at −80 °C until analyzed on the instrument.

Lipid levels were analyzed by liquid chromatography (Shimadzu Nexera X₂ system, Shimadzu Scientific Instrument, Columbia, MD, USA) coupled to electrospray mass spectrometry (QTRAP 6500+, Sciex, Framingham, MA, USA). For each analysis, 5 μL of sample was injected on a BEH C18 1.7 μm, 2.1×100 mm column (Waters Corporation, Milford, Massachusetts, USA) using a flow rate of 0.25 mL/min at 55 °C. For positive ionization mode, mobile phase A consisted of 60:40 acetonitrile/water (v/v) with 10 mM ammonium formate+0.1% formic acid; mobile phase B consisted of 90:10 isopropyl alcohol/acetonitrile (v/v) with 10 mM ammonium formate+0.1% formic acid. For negative ionization mode, mobile phase A consisted of 60:40 acetonitrile/water (v/v) with 10 mM ammonium acetate; mobile phase B consisted of 90:10 isopropyl alcohol/acetonitrile (v/v) with 10 mM ammonium acetate. The gradient was programmed as follows: 0.0-8.0 min from 45% B to 99% B, 8.0-9.0 min at 99% B, 9.0-9.1 min to 45% B, and 9.1-10.0 min at 45% B. Electrospray ionization was performed in either positive or negative ion mode applying the following settings: curtain gas at 30; collision gas set at medium; ion spray voltage at 5500 (positive mode) or 4500 (negative mode); temperature at 250 °C (positive mode) or 600 °C (negative mode); ion source Gas 1 at 50; ion source Gas 2 at 60. Data acquisition was performed using Analyst 1.6.3 (Sciex) in multiple reaction monitoring mode (MRM), with the following parameters: dwell time (msec) and collision energy (CE); declustering potential (DP) at 80; entrance potential (EP) at 10 (positive mode) or −10 (negative mode), and collision cell exit potential (CXP) at 12.5 (positive mode) or −12.5 (negative mode). Lipids were quantified using a mixture of non-endogenous internal standards. Lipids were identified based on their retention times and MRM properties of commercially available reference standards (Avanti Polar Lipids, Birmingham, AL, USA).

FIGS. 8C and 8D illustrate levels of cholesterol ester (CE) (FIG. 8C) and triacylglyceride (TAG) lipid species (FIG. 8D) as detected by mass spectrometry in cell lysates of iPSC microglia cells treated with exemplary anti-TREM2 antibodies for 72 hours after a 24-hour myelin treatment. FIGS. 8E and 8F illustrate levels of cholesterol ester (CE) (FIG. 8E) and triacylglyceride (TAG) lipid species (FIG. 8F) as detected by mass spectrometry in cell lysates of iPSC microglia cells which underwent myelin washout experiments with exemplary anti-TREM2 antibodies. LC/MS data generated in FIGS. 8C-8F were normalized to the internal standards for CE data and normalized to myelin+isotype control for each individual lipid species for TAG data.

Lipid accumulation in iPSC microglia is induced by myelin treatment, which is reflected by an increase in neutral lipid staining (Nile Red) and by LC/MS for detection of specific lipid species in cellular lysates. The data illustrated in FIGS. 8A-8F collectively indicate that treatment of iPSC microglia cells post-myelin challenge with the exemplary anti-TREM2 antibodies reduced accumulation of lipid species, as indicated by the decrease in neutral lipid staining in cells and by the decrease of CE and TAG lipid species levels measured by LC/MS. The reduction of lipid levels as a result of antibody treatment was observed at different timepoints ranging from 24 hours to 72 hours. To eliminate the possibility that the reduction in lipid levels is caused by blocking of lipid uptake, myelin washout experiments in which myelin was removed prior to anti-TREM2 antibody addition were carried out. FIG. 8F illustrates that anti-TREM2 antibodies also reduced lipid levels in iPSC microglia with myelin washout prior to antibody treatment relative to isotype control.

### Example 4. Functional Epitope Binning of Antibodies

TREM2 antibody epitope bins were determined by competition binding on TREM2 protein. Epitope binning experiment was performed on a Carterra LSA instrument using a classical sandwich epitope binning configuration method at 25 °C. All test antibodies were immobilized onto a HC30M chip by amine coupling. Multiple cycles of sandwich competition binding were then carried out for the test antibodies. Each cycle consisted of antigen (His-tagged TREM2 ECD) injection followed by analyte antibody injection to the immobilized antibodies. At the end of each cycle, the surface of immobilized antibodies were regenerated by injecting a low pH buffer (pH=3) containing 1.25 M NaCl. Epitope binding data was evaluated by Carterra software to create competition matrix and epitope bins. Results are provided in Table 2.

Two agonist bins were identified by epitope binning of the anti-TREM2 antibodies: (1) stalk binding agonists, and (2) IgV domain binding agonists. Antibodies within the same bins demonstrated the same function, e.g., inhibition of TREM2-DAP12 pSyk activation by lipid ligand (antagonist antibodies), activation of pSyk with antibody alone (stalk binding agonists, IgV domain binding agonists).

TABLE 2
Anti-TREM2 Antibody Bins, Annotated by Functional Class

| Bin 1 | Bin 2 |
|---|---|
| Stalk Agonist | IgV Agonist |
| CL0020188 | CL0020123 |

### Example 5. Antibody Binding to TREM2 Stalk Peptide

TREM2 antibodies were evaluated for binding to human and mouse TREM2 stalk region peptides. The tested peptides included: (1) full length stalk region (amino acids 129-172 of human TREM2, UniProtKB Q9NZC2; amino acids 131-169 of mouse TREM2, UniProtKB Q99NH8) and (2) a truncated stalk peptide containing the ADAM10/17 cleavage site (amino acids 149-163 of human/mouse TREM2). Antibody binding to TREM2 stalk peptides was detected using standard sandwich ELISA. Briefly, a 96-well half-area ELISA plate was coated with streptavidin overnight at 4 °C. The following day, biotinylated TREM2 stalk peptides diluted in 1% BSA/PBS were added to the plate and incubated for 1 hour. Antibodies diluted in 1% BSA/PBS were then added and incubated for 1 hour. Antibodies bound to peptide were detected with anti-human kappa-RP secondary antibody (Bethyl Laboratories, Inc.) diluted in 1% BSA/PBS. Plates were assayed by reaction with a detection reagent (One-step TMB Ultra, Thermo) and measurement of absorbance at 450 nm (A450) by standard spectrophotometry instrumentation (BioTek®). The results are provided in Table 3, below. Data are shown as normalized values (fold over background where background=isotype control).

TABLE 3
Anti-TREM2 Antibody Binding to TREM2 Stalk Peptides

| | Isotype | CL0020188 | CL0020123 |
|---|---|---|---|
| Human TREM2 peptide binding | 1.0 | 3.0 | 1.2 |
| (aa 149-163) | | | |
| Human TREM2 peptide binding | 1.0 | 51 | 1.2 |
| (aa 129-172) | | | |
| Mouse TREM2 peptide binding | 1.0 | 0.9 | 0.9 |
| (aa 149-163) | | | |
| Mouse TREM2 peptide binding | 1.0 | 33 | 1.0 |
| (aa 131-169) | | | |

Table 3 depicts human and mouse stalk region peptide-antibody binding interactions which support the epitope binning data in Table 2. Based on the data in Table 3, the site at which certain anti-TREM2 antibodies appear to bind corresponds to amino acids 129-148 in the TREM2 extracellular stalk region.

The ability of anti-TREM2 antibodies to inhibit TREM2 stalk peptide cleavage by ADAM17 was also analyzed using a fluorescence polarization assay. TREM2 stalk peptides were first prepared in assay buffer (25 mM Tris pH 7.5, 2.5 μM ZnCl₂, 0.005% Brij-35) with streptavidin. Anti-TREM2 antibodies were then pre-incubated with TREM2 stalk peptides for 30 minutes at room temperature. After the pre-incubation period, ADAM17 (R&D systems, Catalog No. 930-ADB) was added and incubated with the peptides for 20 hours at 37 °C. The following day, samples were further diluted in assay buffer and transferred to a black opaque 384-well plate. Fluorescence polarization was subsequently measured on Perkin Elmer EnVision plate reader. The fluorescence polarization of TREM2 stalk peptides pre-incubated with anti-TREM2 antibody was compared to fluorescence polarization of full-length TREM2 stalk peptide and enzyme control (full-length TREM2 stalk peptide with ADAM17).

TREM2 stalk-binding antibodies significantly increased fluorescence polarization, demonstrating partial inhibition of stalk peptide cleavage by ADAM17 (clones CL0020141, CL0020188, CL0020313, CL0020308). IgV binding antibody CL0020107 did not bind TREM2 stalk region peptide and thus did not show an effect on peptide cleavage in the fluorescence polarization assay.

### Example 6. Pharmacokinetic Analysis of Anti-TREM2 Antibodies

The pharmacokinetic profiles of the anti-TREM2 antibodies were evaluated in mice. C57BL/6J mice were purchased from Jackson Laboratory (Stock No. 000664) at 2 months of age used for a 7-day pharmacokinetic (PK) study and target engagement study. Human Trem2 cDNA KI homozygous mice (huTrem2^{KI/KI}) were used for 24-hour target engagement study at 3 months of age. Generation and breeding of the human Trem2 cDNA KI mice are described below.

Generation of Human Trem2 cDNA KI Mouse Model

A human TREM2 cDNA KI mouse (huTrem2^{KI/KI}) was generated as follows. Human Trem2 cDNA-pA sequence was inserted at the mouse Trem2 endogenous ATG start site. The insertion of human Trem2 cDNA-pA resulted in replacement of the exon1 sequence of mouse Trem2, which allows expression of human Trem2 cDNA driven by the endogenous mouse promoter and disruption of the expression of endogenous mouse Trem2. The huTrem2^{KI/KI} mouse was generated in C57BL/6 genetic background using homologous recombination.

For the huTrem2^{KI/KI} targeting vector, the long homology arm (LA) extends about 3.6 kb upstream of the 5′ end of the human Trem2 cDNA-pA sequence, and the short homology arm (SA) extends about 2.3 kb downstream of the 3′ to the FRT-flanked Neo cassette. Both the long and short homology arms were amplified from a C57BL/6 BAC clone (RP23: 358G22) and then sub-cloned into a ~2.4 kb pSP72 (Promega) backbone vector containing an ampicillin selection cassette. A hUBS-gb2 FRT-flanked Neomycin cassette was inserted immediately downstream of the hTrem2-pA cassette, resulting in a targeting vector of about 13.6 kb in size. Ten (10) μg of the targeting vector was linearized with restriction enzyme Not I (New England Biolabs) and then transfected into FLP C57Bl/6 (B6) embryonic stem (ES) cells by electroporation. After selection with G418 antibiotic, surviving clones were expanded for PCR analysis to identify positive recombinant ES clones. The primer sequences for PCR screening (SEQ ID NO:53=5′-AGG AAT GTG GGG AGC ACG GAG-3′ and SEQ ID NO:54=5′-TGC ATC GCA TTG TCT GAG TAG GTG-3′) amplified a 2.81-kb fragment containing the region from the bghpA element to the downstream of the short homology arm (SA) outside the 3′ region. Five clones were identified as positive and selected for further expansion. The Neo cassette was removed by Flp transgene during ES clone expansion.

Genomic DNA extracted from the five positive clones were first characterized by sequencing analysis. A 1.19-kb product was amplified and sequenced by primers (SEQ ID NO:55=5′-ACC CTA GTC CTG ACT GTT GCT C-3′; SEQ ID NO:56=5′-TAT AGG AAC TTC GCG ACA CGG ACA C-3′) to confirm the 5′ genome/neo cassette junction and 3′ KI cassette junctions. The sequencing results confirmed the introduction of human Trem2 cDNA-pA sequence in all of the five clones.

The five positive clones were further characterized by Southern Blotting analysis using probes targeted against short arm and long arm. ES cell genomic DNA digested with Ssp I and Bam HI were hybridized with short arm and long arm probe respectively. All five ES clones were confirmed to carry the correct homologous recombination events in both long and short arms. The primer sequences for amplifying the short arm probe (658 bp) are (SEQ ID NO:57=5′-ACA GGA GGG ACC TAC CTT CAG 3′; SEQ ID NO:58=5′-GCC TGC CTT TCA GAG ACC TCA GTC-3). The primer sequences for amplifying the long arm probe (681 bp) are (SEQ ID NO:59=5′-CCT CTC CGG CTG CTC ATC TTA CTC-3′; SEQ ID NO:60=5′-GTC TCT CAG CCC TGG CAG AGT TTG-3′).

All five ES cell clones were then injected into C57BL/6 blastocysts. The pups from one clone were confirmed with germline transmission by PCR genotyping. The primers for genotyping are (SEQ ID NO:61=pr1: 5′-CGC CTA CCC TAG TCC TGA CTG TTG-3′, SEQ ID NO:62=pr2: 5′-AAA GCC TAC AGC ATC CTC ACC TC-3′; and SEQ ID NO:63=pr3: 5′-GCA TCA TGG GGT TGT AGA TTC CG-3′). The PCR product for the pr1/pr2 on the wild-type is 658 bp. The PCR product for pr1/pr3 on the KI allele is 469 bp.

### Antibody Dosing and Plasma CSF Collection

For PK analysis, the C57BL/6J mice were dosed with anti-TREM2 antibodies or control IgG at 10 mg/kg through intravenous (IV) tail vein injection (dosing volume of about 200 μL/mouse, n=3 for each group). Blood samples were collected at 1 hour, 24 hours, 4 days, and 7 days after dosing. The blood samples at the first three time points were collected through submandibular bleeding by using 3-mm lancets (GoldenRod animal lancets). The last and terminal blood sample at day 7 was collected through cardiac puncture. Blood was collected in EDTA tubes (Sarstedt Microvette 500 K3E, Catalog No. 201341102), inverted slowly to mix, and centrifuged at 4 °C. The plasma (top) layer was transferred to 1.5-mL Eppendorf tubes and stored at −80 °C until analysis.

For the 24-hour target engagement study, C57BL/6J mice were used to test mouse surrogate anti-TREM2 antibodies, and huTrem2^{KI/KI} mice were used to test human anti-TREM2 antibodies. The mice were dosed with anti-TREM2 antibodies or control IgG at 100 mg/kg through intravenous (IV) tail vein injection (dosing volume of about 200 μL/mouse, n=5 for each group). Blood samples were collected 24 hours before dosing to determine baseline levels of TREM2. Terminal blood and CSF samples were collected 24 hours after dosing. Plasma preparation was prepared as described above. For collection of the CSF sample, a sagittal incision was made at the back of the skull, and subcutaneous tissue and muscles were separated to expose the cisterna magna. A pre-pulled glass capillary tube was used to puncture the cisterna magna and collect the CSF sample. The CSF was then centrifuged at 4 °C to remove blood residue, and the CSF supernatant was transferred to a 0.5-mL Low Protein LoBind Eppendorf tube (Eppendorf, Catalog No. 022431064) for storage at −80 °C until analysis.

### Analysis of In Vivo Anti-TREM2 Antibody Plasma Levels

For anti-TREM2 antibody PK analysis, total antibody concentrations in mouse plasma were quantified using a generic human anti-Fc sandwich ELISA. A 384-well MaxiSorp plate was coated overnight with 1 μg/mL anti-huFc donkey polyclonal (Jackson Immunoresearch). Following incubation with plasma diluted 1:2,000 or 1:20,000 in assay buffer (PBST, 1% BSA), an anti-huFc donkey antibody conjugated to HRP (Jackson Immunoresearch) was added as the detection reagent. Standard curves were generated for each individual antibody, from 2 nM to 2.7 μM using 3-fold dilutions, using a five-parameter logistic regression.

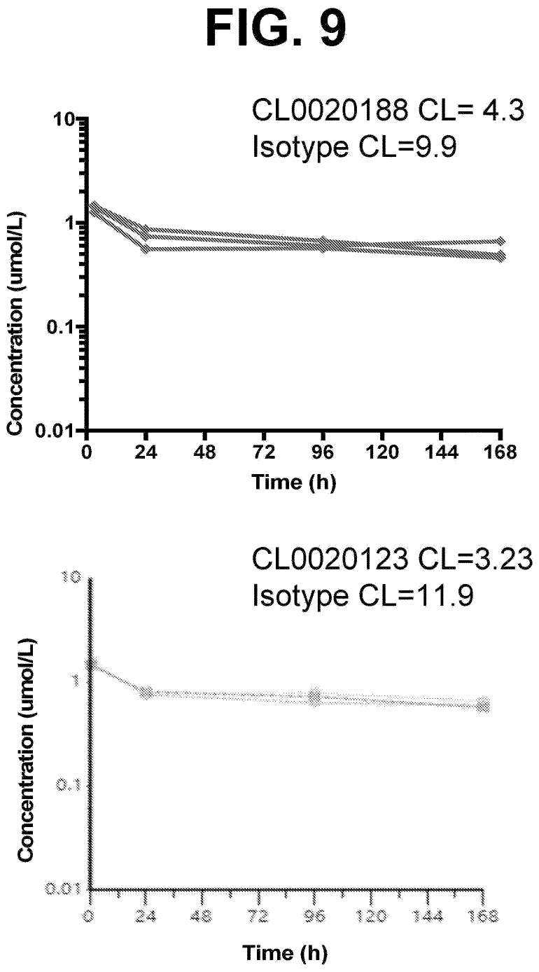

FIG. 9 illustrates representative mouse PK profiles for certain anti-TREM2 antibodies. Antibody clearance rates (CL [mL/day/kg]) are provided for each represented antibody over a 7-day period and are compared to normal effectorless isotype control. Each antibody illustrated in FIG. 9 exhibited comparable clearance rates relative to isotype control.

In Vivo Target Engagement: sTREM2 Plasma Levels

For measurement of soluble TREM2 (sTREM2) plasma levels, human TREM2 cDNA KI mice (huTrem2^{KI/KI}) were bled and then intravenously treated with 100 mg/kg of test anti-TREM2 antibody or isotype control. Mice were bled 24 hours post-dosing. Plasma were obtained from blood samples and evaluated in an MSD assay conducted as follows. MSD SECTOR Plates were coated with 1 μg/mL capture antibody (R+D anti-TREM2 antibody, Catalog No. MAB17291-100) diluted in PBS and incubated overnight at 4 °C. The sample wells were blocked for 1 hour with undiluted MSD Blocker A. Plasma samples were diluted 1:20 in 25% MSD Blocker A in Tris-buffered saline containing 0.05% Tween-20 (TBST) and added to each sample well on the plate, which was then incubated for 2 hours at room temperature. Detection antibody (MSD sulfo-tagged goat anti-human, Catalog No. R32AJ-1, 1:1000) was subsequently added to each sample well, and the plate was incubated for 1 hour at room temperature. TBST washes were performed for each sample well with a Biotek plate washer. Detection reagent (MSD Read buffer) was added and measured using a MSD Meso Sector S600 reader to obtain the results of sTREM2 binding to the antibodies.

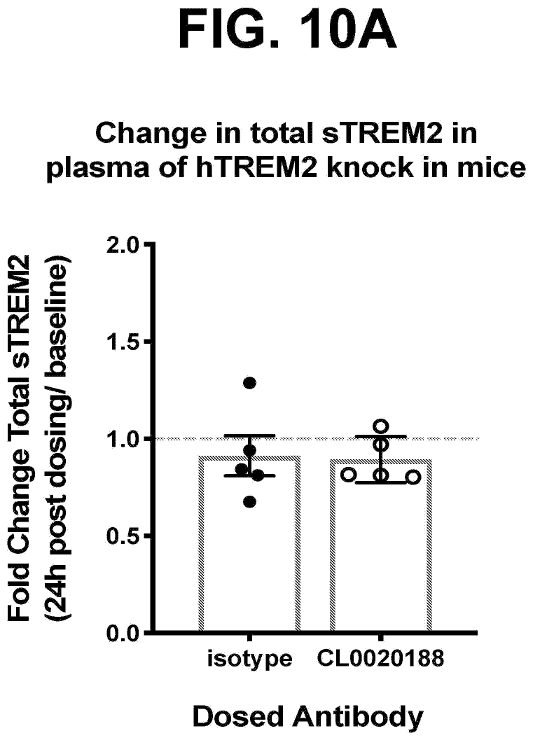

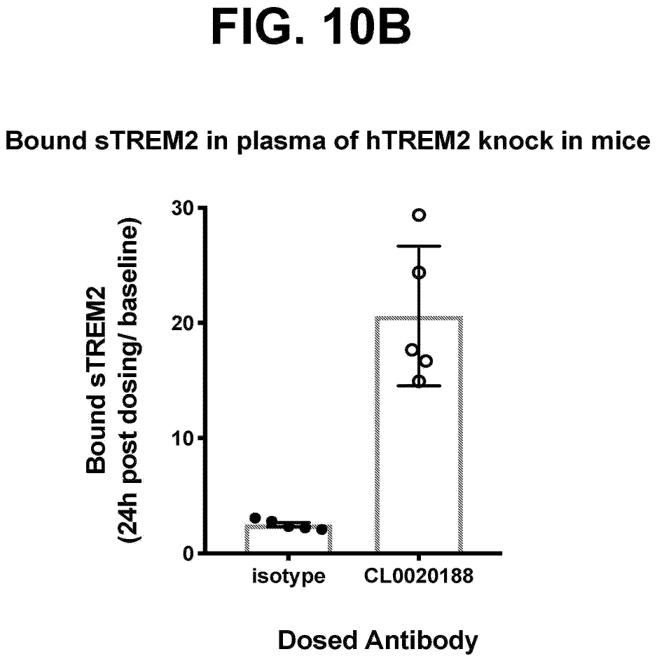

FIGS. 10A and 10B illustrate total sTREM2 levels (FIG. 10A) and antibody-bound sTREM2 levels (FIG. 10B) in huTrem2^{KI/KI} plasma for an exemplary anti-TREM2 antibody. Data were normalized to pre-dose baseline levels of sTREM2 for total and bound sTREM2 assays. The results indicate that total circulating sTREM2 levels did not significantly change between mice treated with anti-TREM2 antibodies compared to isotype control after 24-hour treatment with the antibodies, which suggests that total circulating sTREM2 levels are unaffected at early timepoints post-antibody dose. In contrast, antibody-bound sTREM2 levels were higher in mice injected with anti-TREM2 antibodies relative to isotype control.

### Example 7. Sequence Optimization and Humanization of Anti-TREM2 Antibodies

Exemplary anti-TREM2 antibodies were sequence optimized and humanized, followed by characterization for binding kinetics and binding specificity.

Sequence optimization was conducted by searching within CDR sequences for residues that are susceptible to chemical modification (e.g., asparagine deamidation motifs (NG), aspartic acid isomerization motifs (DS), and potential oxidation residues (tryptophan (W) and methionine (M)) and making amino acid substitutions with conservative and germline residues to remove such sequence liabilities. Humanized and sequence-optimized variants of anti-TREM2 antibodies were then analyzed for binding kinetics using Biacore and dose-titrated cell binding to HEK293-H6 cells (see, Example 1 for representative protocols).

Results for an analysis of the binding characteristics of humanized and sequence-optimized variants of antibody CL0020188 are provided in Table 4. NG motifs in the CL0020188 CDR-H2 sequence (SEQ ID NO:5) and CDR-L1 sequence (SEQ ID NO:7) were modified, grafted onto human framework regions, and analyzed. Table 4 provides K_{D} values as measured by Biacore and EC₅₀ values as measured by dose-titrated binding assay in HEK293-H6 cells.

TABLE 4
Binding Characteristics of Sequence-Optimized
and Humanized Variants of CL0020188

| Clone | hV_{H} | hVL | K_{D} | | EC₅₀ |
|---|---|---|---|---|---|
| CL0020188-1 | NG/graft | NG/graft | 2.3 | nM | 0.42 nM |
| CL0020188-2 | NG/3m | NG/graft | 3.4 | nM | 0.26 nM |
| CL0020188-3 | NG/graft | TG/graft | 6.8 | nM | 0.64 nM |
| CL0020188-4 | NG/3m | TG/graft | 4.8 | nM | 0.44 nM |
| CL0020188-5 | NA/graft | NG/graft | 5.1 | nM | 0.45 nM |
| CL0020188-6 | NA/3m | NG/graft | 4.0 | nM | 0.31 nM |
| CL0020188-7 | NA/graft | TG/graft | 10 | nM | 0.68 nM |
| CL0020188-8 | NA/3m | TG/graft | 7.3 | nM | 0.51 nM |
| Parent | | | 9.5 | nM | 0.44 nM |

3m = A24G/L45P/V48L in V_{H}

As illustrated in Table 4, humanized and sequence-optimized clones of CL0021088 exhibited similar affinity values for hTREM2 compared to the parent antibody (K_{D}=9.5 nM), as measured by Biacore. This was consistent with cell-binding results in HEK293-H6 cells, which are illustrated in Table 4. Compared to the parent antibody (EC₅=0.44 nM), humanized and sequence-optimized clones exhibited comparable and sub-nanomolar affinity for TREM2 expressed in HEK293-H6 cells. Taken together, the results indicate comparable binding kinetics between the parent antibody and the humanized and sequence-optimized variants.

Results for an analysis of the binding characteristics of humanized and sequence-optimized variants of antibody CL0020123 are provided in Table 5. NG and DS motifs in the CL0020123 CDR-H2 sequence (SEQ ID NO:30) were modified, grafted onto human framework regions, and analyzed. Table 5 provides provides K_{D} values as measured by Biacore and EC₅₀ values as measured by dose-titrated binding assay in HEK293-H6 cells.

TABLE 5
Binding Characteristics of Sequence-Optimized
and Humanized Variants of CL0020123

| Clone | hV_{H} | hVL | K_{D} | EC₅₀ |
|---|---|---|---|---|
| CL0020123-1 | NGDS (SEQ ID | Vκ graft | 0.14 nM | 0.25 nM |
| | NO: 80)/2m | | | |
| CL0020123-2 | NGDS (SEQ ID | Vκ graft | 0.18 nM | 0.23 nM |
| | NO: 80)/1m | | | |
| CL0020123-3 | QGDS (SEQ ID | Vκ graft | 0.40 nM | 0.24 nM |
| | NO: 81)/2m | | | |
| CL0020123-4 | NGES (SEQ ID | Vκ graft | 0.17 nM | 0.17 nM |
| | NO: 82)/2m | | | |
| CL0020123-5 | QGES (SEQ ID | Vκ graft | 0.44 nM | 0.37 nM |
| | NO: 83)/2m | | | |
| CL0020123-6 | QGDS (SEQ ID | Vκ graft | 0.32 nM | 0.29 nM |
| | NO: 81)/1m | | | |
| CL0020123-7 | NGES (SEQ ID | Vκ graft | 0.15 nM | 0.19 nM |
| | NO: 82)/1m | | | |
| CL0020123-8 | QGES (SEQ ID | Vκ graft | 0.39 nM | 0.38 nM |
| | NO: 83)/1m | | | |
| Parent | | | 0.10 nM | 0.26 nM |

1m = R71A in V_{H}
2m = V67A/R71A in V_{H}

Compared to the parent antibody (K_{D}=0.10 nM), the humanized and sequence-optimized clones exhibited about 4-fold higher K_{D} values for binding to hTREM2 as measured by Biacore. On the other hand, in dose-titrated cell-binding assays in HEK293-H6 cells, the humanized and sequence-optimized clones exhibited comparable and sub-nanomolar affinity for TREM2.

### Example 8. In Vitro Characterization of Sequence-Optimized, Humanized Anti-TREM2 Antibodies

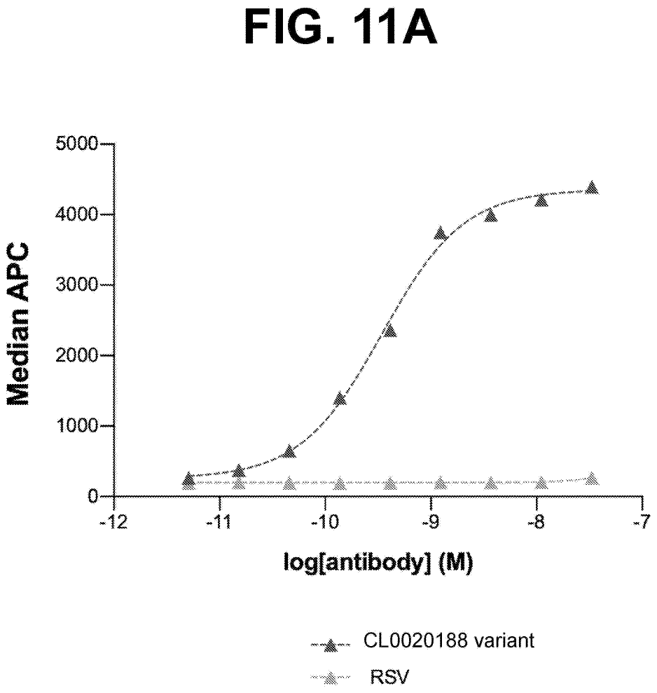

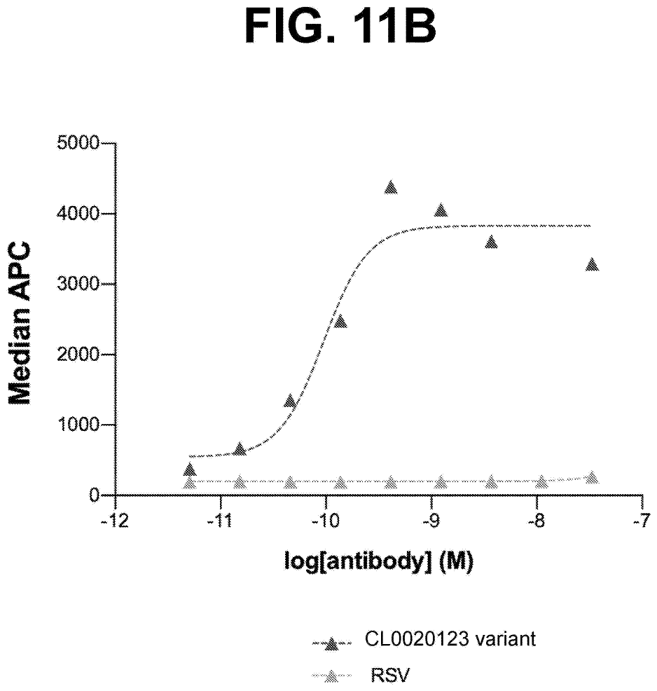

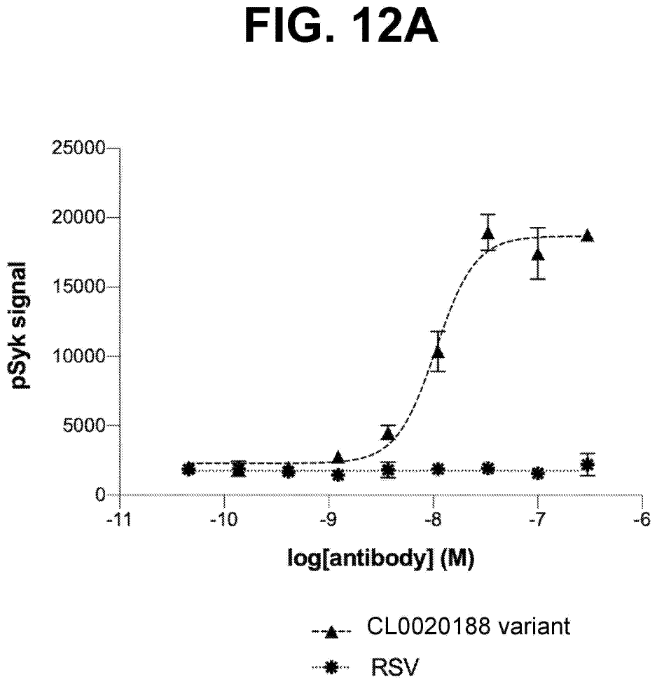

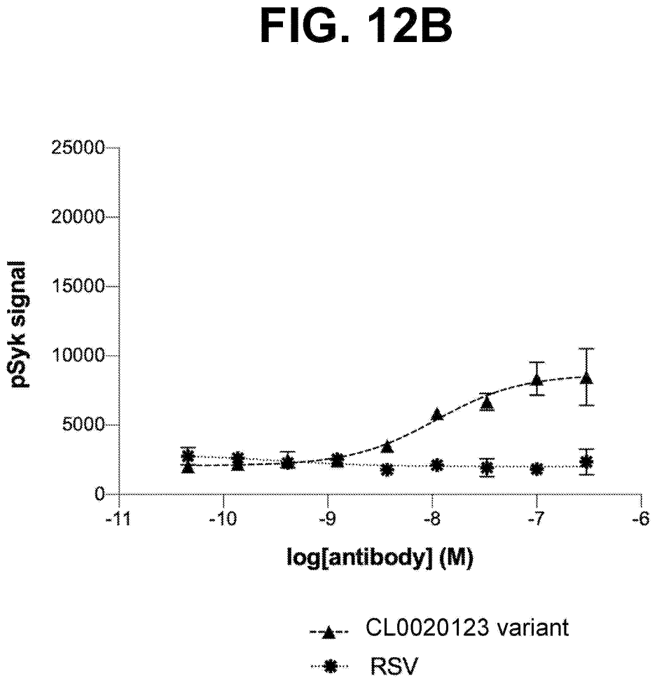

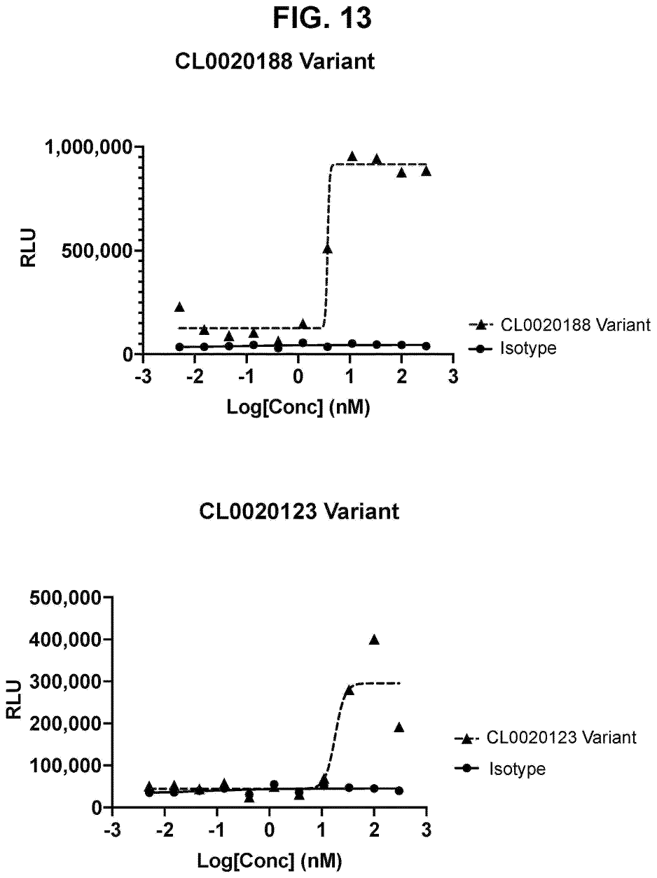

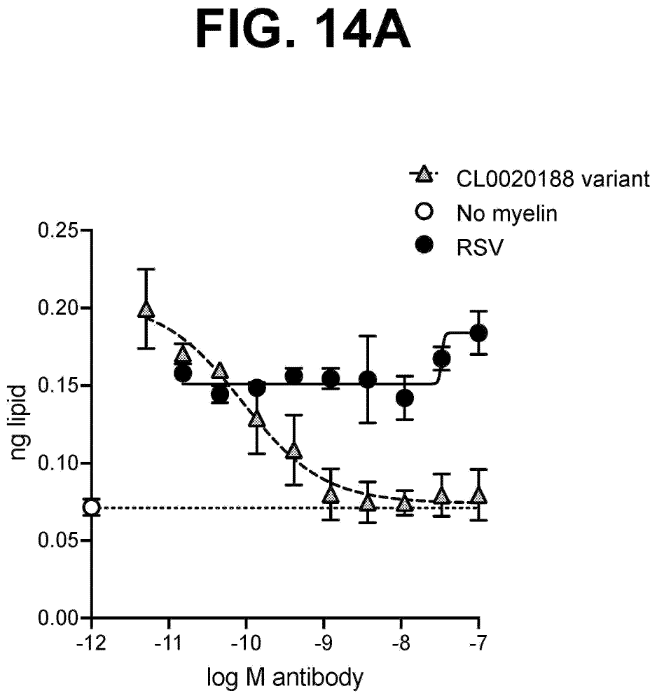

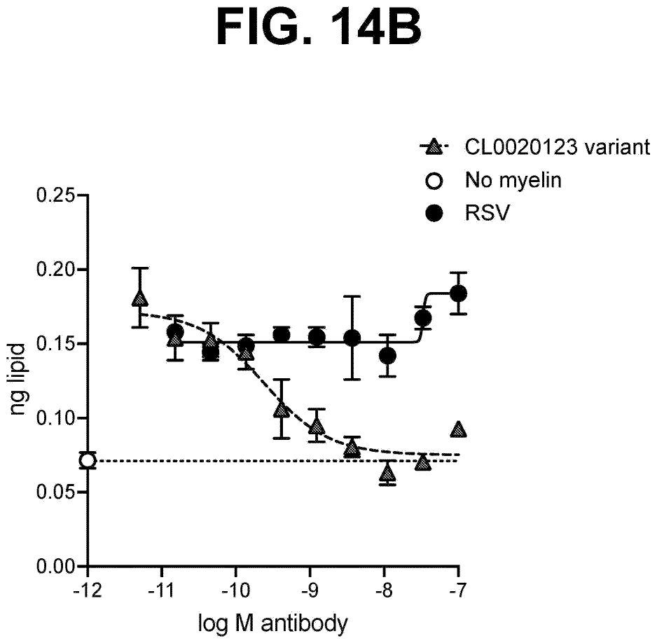

Exemplary sequence-optimized and humanized anti-TREM2 antibodies (Example 7) were evaluated by in vitro methods as described in Examples 1 and 3. The antibodies were assessed for TREM2 binding in TREM2-expressing HEK cells, TREM2-dependent pSyk signaling in HEK-H6 cells, capability for promoting survival of human macrophage cells, and ability to modulate lipid accumulation in iPSC microglia. FIGS. 11 to 14 illustrate the results for representative anti-TREM2 antibodies. The anti-TREM2 antibodies exhibited good cell binding in assays with TREM2-expressing HEK293-H6 cells (EC50 values of 0.34 nM (FIG. 11A) and 0.08 nM (FIG. 11B). The anti-TREM2 antibodies also activated pSyk signaling in TREM2-expressing HEK293-H6 cells (FIGS. 12A and 12B). In addition, the anti-TREM2 antibodies induced macrophage survival, with better survival activity observed for a CL0020188 variant antibody (FIG. 13). Finally, the anti-TREM2 antibodies demonstrated capability in reducing lipid accumulation in myelin-treated iPSC microglia (FIGS. 14A and 14B).

### Example 9. Mouse PK of Sequence-Optimized, Humanized Anti-TREM2 Antibodies

The pharmacokinetic profiles of certain sequence-optimized, humanized anti-TREM2 antibodies (Example 7) were evaluated in wild-type mice for a 7-day pharmacokinetic (PK) study similar to that described in Example 6. Each dose group contained n=3 mice. Table 6 provides the PK properties of exemplary sequence-optimized and humanized anti-TREM2 antibodies (Example 7).

TABLE 6
Mouse PK Properties of Anti-TREM2 Antibodies

| | Dose | C₀ | AUC_{0-inf} | CL |
|---|---|---|---|---|
| Clone | (mg/kg) | (μM) | (μM*hr) | (mL/day/kg) |
| CL0020188 | 10 | 1.14 ± 0.05 | 207.78 ± 43.30 | 8.14 ± 1.80 |
| variant | | | | |
| CL0020188 | 50 | 6.48 ± 0.61 | 236.49 ± 2.67 | 34.58 ± 0.39 |
| variant | | | | |
| CL0020123 | 10 | 1.4999 | 223.1 | 13.2 |
| variant | | | | |
| Anti-BACE | 10 | 1.017 | 60.19 | 33.3 |
| reference | | | | |

### Example 10. Cyno PK of Sequence-Optimized, Humanized Anti-TREM2 Antibodies

The pharmacokinetic profiles of the anti-TREM2 antibodies were evaluated in naïve cynomolgus monkeys. Naïve cynomolgus monkeys of 2-4 years of age (approximately 2-3 kg in weight) were injected with anti-TREM2 antibody via intravenous bolus injection. Administered doses included 3 mg/kg and 25 mg/kg, with n=3 monkeys per dose group. Blood samples (about 1 mL) were collected pre-dose and at 10 minutes, 30 minutes, at 1, 6, 12, and 24 hours, and at 3, 7, 10, 14, 17, 21, 24, and 28 days post-dose. The samples were chilled at about 5 °C prior to centrifugation to obtain plasma, and the obtained plasma was maintained on dry ice prior to storage at −70 °C until analysis. The plasma samples were analyzed for anti-TREM2 antibody levels as follows.

For anti-TREM2 antibody PK analysis, the total antibody concentrations in monkey plasma were quantified using a generic anti-human_IgG sandwich electrochemiluminescence immunoassay (ECLIA) on a Meso Scale Discovery (MSD) platform. Briefly, 1% casein-based PBS blocking buffer (Thermo Scientific, MA) was added to an MSD GOLD 96-well small-spot streptavidin-coated microtiter plate (Meso Scale Discovery, MD) and incubated for approximately 1 hr. Following the plate blocking and wash steps, a biotinylated anti-human_IgG goat antibody (SouthemBiotech, AL) at a working solution of 0.5 μg/mL was added to the assay plate and allowed to incubate for 1-2 hrs. Following the incubation and wash steps, plasma test samples (i.e., samples with anti-TREM2 humanized antibodies) were added to the assay plate and incubated for 1-2 hrs. Note that the test samples must be diluted at the assay minimum-required-dilution (MRD) of 1:100 in 0.5% casein-based PBS assay buffer (Thermo Scientific, MA), resulting in the final 1% plasma matrix, prior to adding to the assay plate. Following the capture of anti-TREM2 antibody analyte and wash steps, a secondary ruthenylated (SULFO-TAG) anti-human IgG goat antibody (Meso Scale Discovery, MD) at a working solution of 0.4 μg/mL was added to the assay plate and incubated for approximately 1 hr. Lastly, following the incubation and wash steps, an assay read buffer (1×MSD Read Buffer T) was added to the assay plate to generate assay sample signals. The sample signal readouts from an MSD plate reader was in a form of electrochemiluminescence (ECL) signals and expressed in ECL units (ECLU). All of the assay reaction steps previously described were done at the ambient temperature and with shaking on a plate shaker (where appropriate). The assay had a dynamic calibration standard range of 19.5-2500 ng/mL (or 0.195-25 ng/mL in post-MRD of 1:100) with 8 standard points serially-diluted at 1:2 plus a blank plasma sample. Plasma sample concentration was back-calculated off the assay calibration standard curve which was fitted with a weighed four-parameter non-linear logistic regression. Table 7 provides the pharmacokinetic (PK) properties of an exemplary sequence-optimized and humanized anti-TREM2 antibody (Example 7).

TABLE 7
Cynomolgus PK Properties of Anti-TREM2 Antibodies

| | Dose | AUC_{0-inf} | CL |
|---|---|---|---|
| Clone | (mg/kg) | (uM*hr) | (mL/day/kg) |
| CL0020188 variant | 25 | 899.0 ± 53 | 4.46 ± 0.26 |
| CL0020188 variant | 3 | 61.2 ± 6.3 | 7.90 ± 0.77 |

The CL0020188 variant exhibited similar low clearance levels between different dose levels and a linear pharmacokinetic profile in cynomolgus monkeys. In addition, there were no clinical pathology findings related to administration of the variant in cynomolgus monkeys (data not shown).

### Example 11. Comparison of Anti-TREM2 Antibodies

The affinity of anti-TREM2 antibodies to human TREM2 and cynomolgus TREM2 were measured by Biacore (described in Example 1). The potency of anti-TREM2 antibodies were measured in healthy human volunteer CSF samples (Innovative Research) and healthy cynomolgus monkey CSF samples (Worldwide Primates), by MSD assay. The potency of each antibody was determined by its EC50. Briefly, MSD GOLD 96-well small spot streptavidin plates (MSD, Cat. No. L45SA) coated with capture antibody (biotinylated goat anti-human TREM2, R&D Systems, Cat. No. BAF1828) was incubated with biofluid samples diluted 1:3 in Assay Buffer (25% (v/v) MSD Blocker A (MSD, Cat. No. R93BA-A), 75% (v/v) TBST) for one hour at room temperature. After rinsing the wells with TBST, sulfo-tagged anti-TREM2 antibody was added in a serial dilution (4× dilution over 11 points) to the wells of the plate and incubated for one hour at room temperature. The wells were washed with TBST, and MSD Read Buffer (MSD, Cat. No. R92TC-3) was added to the wells. Signal from the samples were measured using an MSD Meso Sector S600 device. EC50 values for each antibody was determined by four-parameter variable slope non-linear regression. Reference antibodies #1 and #2 correspond to 4C5 and 6E7 described in WO 2018/195506. Reference antibody #3 corresponds to AL2p-58 described in WO 2019/028292. The variable regions of reference antibody #1 are represented by SEQ ID NOS:73 and 74. The variable regions of reference antibody #2 are represented by SEQ ID NOS:75 and 76. The variable regions of reference antibody #3 are represented by SEQ ID NOS:77 and 78. Results are provided in Table 8.

As illustrated in Table 8, the CL0020188 and CL0020123 variants as disclosed herein, relative to reference antibodies, have stronger affinity for human TREM2 and are more potent in binding sTREM2 in CSF samples isolated from healthy human volunteer subjects. In addition, the CL0020188 variants, relative to reference antibodies, have stronger affinity for cynomolgus TREM2 and are more potent in binding sTREM2 in CSF samples isolated from healthy cynomolgus monkey subjects. This is demonstrated by the relative amount of antibody needed to reach the half maximal effective concentration for binding a given amount of sTREM2 under the same conditions (EC50). As illustrated in Table 8, higher relative amounts of reference antibodies #1, #2, and #3 are needed to achieve EC50 compared to the CL0020188 and CL0020123 antibodies.

TABLE 8
Comparison of Properties of Anti-TREM2 Antibodies

| | | | EC50, bound | EC50, bound |
|---|---|---|---|---|
| | K_{D} (nM) | K_{D} (nM) | sTREM2 in | sTREM2 in |
| | Human | Cyno | Human CSF | Cyno CSF |
| Antibody | TREM2 | TREM2 | [nM] | [nM] |
| CL0020188-4 | 1.4 | 1.5 | 0.29 | 0.28 |
| CL0020188-7 | 3.3 | 2.8 | 0.75 | 0.72 |
| CL0020123-5 | 0.63 | 9.3 | 1.00 | 49.41 |
| CL0020123-7 | 0.08 | 9.4 | 0.54 | 16.88 |
| Reference | 5.9 | 14 | 32.6 | 81.6 |
| antibody #1 | | | | |
| Reference | 4.3 | 9.2 | 7.4 | 6.5 |
| antibody #2 | | | | |
| Reference | 8.7 | 9.2 | 7.25 | 33.72 |
| antibody #3 | | | | |

### Example 12. Epitope Mapping of Anti-TREM2 Antibodies

To identify the epitopes of the anti-TREM2 antibodies at the peptide level, hydrogen deuterium exchange (HDX) mass spectrometry was used. Recombinant human TREM2 alone or mixed with anti-TREM2 antibody was incubated with deuterium oxide labeling buffer (50 mM sodium phosphate, 100 mM sodium chloride, pH 7.0) for 0, 60, 600, and 3600 seconds at 20 °C. Hydrogen/deuterium exchange was quenched by adding an equal volume of 4 M guanidine hydrochloride, 0.85 M TCEP buffer (final pH 2.5). The quenched samples were then subjected to pepsin/protease XIII digestion and LC-MS analysis. Briefly, the quenched samples were injected onto a packed pepsin/protease XIII column (2.1×30 mm) held at 20 °C, and the resultant peptides were analyzed using an UPLC-MS system comprised of a Waters Acquity UPLC (Waters Corporation) coupled to a Q Exactive™ HF-Hybrid Quadrupole-Orbitrap Mass Spectrometer (Thermo Fisher). The peptides were separated on a 50 mm×1 mm C8 column held at −6 °C using a 16.5-minute gradient from 2% to 31% solvent B (solvent B: 0.2% formic acid in acetonitrile; solvent A: 0.2% formic acid in water). The mass spectra were recorded in MS only mode. Raw MS data was processed using HDX Workbench (Pascal et al. 2012. Journal of The American Society for Mass Spectrometry 23:1512-1521). The deuterium levels were calculated using the average mass difference between the deuterated peptide and its native form at t0. Peptide identification was carried out through searching MS/MS data against the human TREM2 sequence with Mascot softward (Matrix Science). The mass tolerance for precursor and product ions were 10 ppm and 0.02 Da, respectively.

Based on the HDX mass spectrometry results, CL0020188 and variants of CL0020188 bind to human TREM2 (SEQ ID NO:1) at amino acid residues 143-149 (FPGESES (SEQ ID NO:69)), while CL0020123 and variants of CL0020123 bind to human TREM2 at (i) amino acid residues 55-63 (GEKGPCQRV (SEQ ID NO:70)), (ii) amino acids 96-107 (TLRNLQPHDAGL (SEQ ID NO:71)), and (iii) amino acid residues 126-129 (VEVL (SEQ ID NO:72)).

### IX. Informal Sequence Listing

| SEQ | | |
|---|---|---|
| ID NO | Sequence | Description |
| 1 | MEPLRLLILLFVTELSGAHNTTVFQGVAGQSLQVSCPYDSMKH | Human TREM2 protein |
| | WGRRKAWCRQLGEKGPCQRVVSTHNLWLLSFLRRWNGSTAIT | |
| | DDTLGGTLTITLRNLQPHDAGLYQCQSLHGSEADTLRKVLVEVL | |
| | ADPLDHRDAGDLWFPGESESFEDAHVEHSISRSLLEGEIPFPPTSI | |
| | LLLLACIFLIKILAASALWAAAWHGQKPGTHPPSELDCGHDPGY | |
| | QLQTLPGLRDT | |
| 2 | EVKLLDSGGGLVQAGGSLRLSCAGSGFTFTDFYMSWIRQPPGKA | CL0020306 V_{H} |
| | PEWLGVIRNKANGYTAGYNPSVKGRFTISRDNTQNILYLQMNTL | |
| | RAEDTAIYYCARLSYGFDYWGQGVMVTVSS | |
| 3 | DIVMTQGALPNPVPSGESASITCQSSKSLLHSNGKTYLNWYLQR | CL0020306 VL |
| | PGQSPQLLIYWMSTRASGVSDRFSGSGSGTDFTLKISSVEAEDVG | |
| | VYYCQQFLEFPFTFGSGTKLEIK | |
| 4 | GFTFTDFYMS | CL0020306 CDR-H1; CDR- |
| | | H1 for CL0020188 and |
| | | variants CL0020188-1, |
| | | CL0020188-2, CL0020188-3, |
| | | CL0020188-4, CL0020188-5, |
| | | CL0020188-6, CL0020188-7, |
| | | and CL0020188-8 |
| 5 | VIRNKANGYTAGYNPSVKG | CL0020306 CDR-H2; CDR- |
| | | H2 for CL0020188 and |
| | | variants CL0020188-1, |
| | | CL0020188-2, CL0020188-3, |
| | | and CL0020188-4 |
| 6 | ARLSYGFDY | CL0020306 CDR-H3 |
| 7 | QSSKSLLHSNGKTYLN | CL0020306 CDR-L1; |
| | | CL0020307 CDR-L1; |
| | | CL0020307-1 CDR-L1; |
| | | CDR-L1 for CL0020188 and |
| | | variants CL0020188-1, |
| | | CL0020188-2, CL0020188-5, |
| | | and CL0020188-6 |
| 8 | WMSTRAS | CL0020306 CDR-L2; |
| | | CL0020307 CDR-L2; |
| | | CL0020307-1 CDR-L2; |
| | | CDR-L2 for CL0020188 and |
| | | variants CL0020188-1, |
| | | CL0020188-2, CL0020188-3, |
| | | CL0020188-4, CL0020188-5, |
| | | CL0020188-6, CL0020188-7, |
| | | and CL0020188-8 |
| 9 | QQFLEFPFT | CL0020306 CDR-L3; |
| | | CL0020307 CDR-L3; |
| | | CL0020307-1 CDR-L3 |
| 10 | EVKLLESGGGLVQPGGSLRLSCAASGFTFTNFYMSWIRQPPGRA | CL0020307 V_{H} |
| | PEWLGVIRNRPNGYTTDYNPSVKGRFTISRDNTQNILYLQMSTL | |
| | RADDTAFYYCTRLTYGFDYWGQGVMVTVSS | |
| 11 | DIVMTQGALPNPVPSGESASITCQSSKSLLHSNGKTYLNWYLQR | CL0020307 VL |
| | PGQSPQLLIYWMSTRASGVSDRFSGSGSGTDFTLKISSVEAEVVG | |
| | VYYCQQFLEFPFTFGSGTKLEIK | |
| 12 | GFTFTNFYMS | CL0020307 CDR-H1 |
| 13 | VIRNRPNGYTTDYNPSVKG | CL0020307 CDR-H2 |
| 14 | TRLTYGFDY | CL0020307 CDR-H3 |
| 15 | EVKLLDSGGGLVQAGGSLRLSCAGSGFTFTDFYMSWIRQPPGKA | CL0020188 V_{H} |
| | PEWLGVIRNKANGYTAGYNPSVKGRFTISRDNTQNILYLQMNTL | |
| | RAEDTAIYYCARLTYGFDYWGQGVMVTVSS | |
| 16 | DIVMTQGALPNPVPSGESASITCQSSKSLLHSNGKTYLNWYLQR | CL0020188 VL |
| | PGQSPQLLIYWMSTRASGVSDRFSGSGSGTDFTLKISSVEAEDVG | |
| | VYYCQQFLEYPFTFGSGTKLEIK | |
| 17 | ARLTYGFDY | CDR-H3 for CL0020188 and |
| | | variants CL0020188-1, |
| | | CL0020188-2, CL0020188-3, |
| | | CL0020188-4, CL0020188-5, |
| | | CL0020188-6, CL0020188-7, |
| | | and CL0020188-8 |
| 18 | QQFLEYPFT | CDR-L3 for CL0020188 and |
| | | variants CL0020188-1, |
| | | CL0020188-2, CL0020188-3, |
| | | CL0020188-4, CL0020188-5, |
| | | CL0020188-6, CL0020188-7, |
| | | and CL0020188-8 |
| 19 | EVQLVESGGGLVQPGGSLRLSCAASGFTFTDFYMSWVRQAPGK | CL0020188-1 V_{H}; |
| | GLEWVSVIRNKANGYTAGYNPSVKGRFTISRDNSKNTLYLQMN | CL0020188-3 V_{H} |
| | SLRAEDTAVYYCARLTYGFDYWGQGTLVTVSS | |
| 20 | DIVMTQTPLSLPVTPGEPASISCQSSKSLLHSNGKTYLNWYLQKP | CL0020188-1 VL; |
| | GQSPQLLIYWMSTRASGVPDRFSGSGSGTDFTLKISRVEAEDVG | CL0020188-2 VL; |
| | VYYCQQFLEYPFTFGQGTKVEIK | CL0020188-5 VL; |
| | | CL0020188-6 VL |
| 21 | EVQLVESGGGLVQPGGSLRLSCAGSGFTFTDFYMSWVRQAPGK | CL0020188-2 V_{H} |
| | GLEWVSVIRNKANGYTAGYNPSVKGRFTISRDNSKNTLYLQMN | |
| | SLRAEDTAVYYCARLTYGFDYWGQGTLVTVSS | |
| 22 | DIVMTQTPLSLPVTPGEPASISCQSSKSLLHSTGKTYLNWYLQKP | CL0020188-3 VL; |
| | GQSPQLLIYWMSTRASGVPDRFSGSGSGTDFTLKISRVEAEDVG | CL0020188-4 VL; |
| | VYYCQQFLEYPFTFGQGTKVEIK | CL0020188-7 VL; |
| | | CL0020188-8 VL |
| 23 | QSSKSLLHSTGKTYLN | CDR-L1 for variants |
| | | CL0020188-3, CL0020188-4, |
| | | CL0020188-7, and |
| | | CL0020188-8 |
| 24 | EVQLVESGGGLVQPGGSLRLSCAASGFTFTDFYMSWVRQAPGK | CL0020188-5 V_{H}; |
| | GLEWVSVIRNKANAYTAGYNPSVKGRFTISRDNSKNTLYLQMN | CL0020188-7 V_{H} |
| | SLRAEDTAVYYCARLTYGFDYWGQGTLVTVSS | |
| 25 | VIRNKANAYTAGYNPSVKG | CDR-H2 for variants |
| | | CL0020188-5, CL0020188-6, |
| | | CL0020188-7, and |
| | | CL0020188-8 |
| 26 | EVQLVESGGGLVQPGGSLRLSCAGSGFTFTDFYMSWVRQAPGK | CL0020188-6 V_{H}; |
| | GPEWLSVIRNKANAYTAGYNPSVKGRFTISRDNSKNTLYLQMN | CL0020188-8 V_{H} |
| | SLRAEDTAVYYCARLTYGFDYWGQGTLVTVSS | |
| 27 | EVQLQQSGAELVRSGASVKLSCTASGFSIEDFYIHWVKQRPEQG | CL0020123 V_{H} |
| | LEWIGWIDPENGDSKYAPKFQGKATMTADTSSNTAYLHLSSLTS | |
| | EDTAVYYCHADHGNYGSTMDYWGQGTSVTVSS | |
| 28 | DIQMNQSPSSLSASLGDTVTITCHASQHINVWLSWYQQKPGDHP | CL0020123 VL |
| | KLLIYKASNLHTGVPSRFSGSGSGTGFTLTISSLQPEDIATYYCQQ | |
| | GQTYPRTFGGGTKLEIK | |
| 29 | GFSIEDFYIH | CDR-H1 for CL0020123 and |
| | | variants CL0020123-1, |
| | | CL0020123-2, CL0020123-3, |
| | | CL0020123-4, CL0020123-5, |
| | | CL0020123-6, CL0020123-7, |
| | | and CL0020123-8 |
| 30 | WIDPENGDSKYAPKFQG | CDR-H2 for CL0020123 and |
| | | variants CL0020123-1 and |
| | | CL0020123-2 |
| 31 | HADHGNYGSTMDY | CDR-H3 for CL0020123 and |
| | | variants CL0020123-1, |
| | | CL0020123-2, CL0020123-3, |
| | | CL0020123-4, CL0020123-5, |
| | | CL0020123-6, CL0020123-7, |
| | | and CL0020123-8 |
| 32 | HASQHINVWLS | CDR-L1 for CL0020123 and |
| | | variants CL0020123-1, |
| | | CL0020123-2, CL0020123-3, |
| | | CL0020123-4, CL0020123-5, |
| | | CL0020123-6, CL0020123-7, |
| | | and CL0020123-8 |
| 33 | KASNLHT | CDR-L2 for CL0020123 and |
| | | variants CL0020123-1, |
| | | CL0020123-2, CL0020123-3, |
| | | CL0020123-4, CL0020123-5, |
| | | CL0020123-6, CL0020123-7, |
| | | and CL0020123-8 |
| 34 | QQGQTYPRT | CDR-L3 for CL0020123 and |
| | | variants CL0020123-1, |
| | | CL0020123-2, CL0020123-3, |
| | | CL0020123-4, CL0020123-5, |
| | | CL0020123-6, CL0020123-7, |
| | | and CL0020123-8 |
| 35 | QVQLVQSGAEVKKPGASVKVSCKASGFSIEDFYIHWVRQAPGQ | CL0020123-1 V_{H} |
| | GLEWMGWIDPENGDSKYAPKFQGRATITADTSTSTAYMELSSL | |
| | RSEDTAVYYCHADHGNYGSTMDYWGQGTLVTVSS | |
| 36 | DIQMTQSPSSLSASVGDRVTITCHASQHINVWLSWYQQKPGKAP | CL0020123-1 VL; |
| | KLLIYKASNLHTGVPSRFSGSGSGTDFTLTISSLQPEDFATYYCQ | CL0020123-2 VL; |
| | QGQTYPRTFGQGTKVEIK | CL0020123-3 VL; |
| | | CL0020123-4 VL |
| | | CL0020123-5 VL; |
| | | CL0020123-6 VL; |
| | | CL0020123-7 VL; |
| | | CL0020123-8 VL |
| 37 | QVQLVQSGAEVKKPGASVKVSCKASGFSIEDFYIHWVRQAPGQ | CL0020123-2 V_{H} |
| | GLEWMGWIDPENGDSKYAPKFQGRVTITADTSTSTAYMELSSL | |
| | RSEDTAVYYCHADHGNYGSTMDYWGQGTLVTVSS | |
| 38 | QVQLVQSGAEVKKPGASVKVSCKASGFSIEDFYIHWVRQAPGQ | CL0020123-3 V_{H} |
| | GLEWMGWIDPEQGDSKYAPKFQGRATITADTSTSTAYMELSSL | |
| | RSEDTAVYYCHADHGNYGSTMDYWGQGTLVTVSS | |
| 39 | WIDPEQGDSKYAPKFQG | CDR-H2 for variants |
| | | CL0020123-3 and |
| | | CL0020123-6 |
| 40 | QVQLVQSGAEVKKPGASVKVSCKASGFSIEDFYIHWVRQAPGQ | CL0020123-4 V_{H} |
| | GLEWMGWIDPENGESKYAPKFQGRATITADTSTSTAYMELSSLR | |
| | SEDTAVYYCHADHGNYGSTMDYWGQGTLVTVSS | |
| 41 | WIDPENGESKYAPKFQG | CDR-H2 for variants |
| | | CL0020123-4 and |
| | | CL0020123-7 |
| 42 | QVQLVQSGAEVKKPGASVKVSCKASGFSIEDFYIHWVRQAPGQ | CL0020123-5 V_{H} |
| | GLEWMGWIDPEQGESKYAPKFQGRATITADTSTSTAYMELSSLR | |
| | SEDTAVYYCHADHGNYGSTMDYWGQGTLVTVSS | |
| 43 | WIDPEQGESKYAPKFQG | CDR-H2 for variants |
| | | CL0020123-5 and |
| | | CL0020123-8 |
| 44 | QVQLVQSGAEVKKPGASVKVSCKASGFSIEDFYIHWVRQAPGQ | CL0020123-6 V_{H} |
| | GLEWMGWIDPEQGDSKYAPKFQGRVTITADTSTSTAYMELSSL | |
| | RSEDTAVYYCHADHGNYGSTMDYWGQGTLVTVSS | |
| 45 | QVQLVQSGAEVKKPGASVKVSCKASGFSIEDFYIHWVRQAPGQ | CL0020123-7 V_{H} |
| | GLEWMGWIDPENGESKYAPKFQGRVTITADTSTSTAYMELSSLR | |
| | SEDTAVYYCHADHGNYGSTMDYWGQGTLVTVSS | |
| 46 | QVQLVQSGAEVKKPGASVKVSCKASGFSIEDFYIHWVRQAPGQ | CL0020123-8 V_{H} |
| | GLEWMGWIDPEQGESKYAPKFQGRVTITADTSTSTAYMELSSLR | |
| | SEDTAVYYCHADHGNYGSTMDYWGQGTLVTVSS | |
| 47 | W-I-D-P-E-X₆-G-X₈-S-K-Y-A-P-K-F-Q-G, wherein X₆ is N or | CDR-H2 consensus sequence |
| | Q and X₈ is D or E | |
| 48 | G-F-T-F-T-B-F-Y-M-S | CDR-H1 consensus sequence |
| 49 | V-I-R-N-X₅-X₆-N-X₈-Y-T-X₁₁-X₁₂-Y-N-P-S-V-K-G, wherein X₅ | CDR-H2 consensus sequence |
| | is K or R; X₆ is A or P; X₈ is G or A; X₁₁ is A or T; and | |
| | X₁₂ is G or D | |
| 50 | X₁-R-L-X₄-Y-G-F-D-Y, wherein X₁ is A or T; and X₄ is T | CDR-H3 consensus sequence |
| | or S | |
| 51 | Q-S-S-K-S-L-L-H-S-X₁₀-G-K-T-Y-L-N, wherein X₁₀ is N or T | CDR-L1 consensus sequence |
| 52 | Q-Q-F-L-E-X₆-P-F-T, wherein X₆ is Y or F | CDR-L3 consensus sequence |
| 53 | AGGAATGTGGGGAGCACGGAG | Primer sequence |
| 54 | TGCATCGCATTGTCTGAGTAGGTG | Primer sequence |
| 55 | ACCCTAGTCCTGACTGTTGCTC | Primer sequence |
| 56 | TATAGGAACTTCGCGACACGGACAC | Primer sequence |
| 57 | ACAGGAGGGACCTACCTTCAG | Primer sequence |
| 58 | GCCTGCCTTTCAGAGACCTCAGTC | Primer sequence |
| 59 | CCTCTCCGGCTGCTCATCTTACTC | Primer sequence |
| 60 | GTCTCTCAGCCCTGGCAGAGTTTG | Primer sequence |
| 61 | CGCCTACCCTAGTCCTGACTGTTG | Primer sequence |
| 62 | AAAGCCTACAGCATCCTCACCTC | Primer sequence |
| 63 | GCATCATGGGGTTGTAGATTCCG | Primer sequence |
| 64 | GGGGS | Linker sequence |
| 65 | HHHHHH | 6X-His tag |
| 66 | QVQLQQPGAELVKPGASVKLSCKASGYTFTSYWMHWVKQSPG | V_{H} for anti-TREM2 antibody |
| | RGLEWIGRSDPTTGGTNYNEKFKTKATLTVDKPSSTAYMQLSSL | RS9.F6 |
| | TSDDSAVYYCVRTSGTGDYWGQGTSLTVSSAKTTAPSVYPLAP | |
| | VCGGTTGSSVT | |
| 67 | DVVMTQTPLSLPVSLGDQASISCRSSQSLVHNNGNTFLHWYLQK | VL for anti-TREM2 antibody |
| | PGQSPKLLIYKVSNRFSGVPDRFSGSGSGTDFTLKISRVEAEDLG | RS9.F6 |
| | VYFCSQTTHVPPTFGGGTKLEIKRADAAPTVSIFPPSSEQLTSGGA | |
| | SVVCF | |
| 68 | DIVMTQSPDSLAVSLGERATINCQSSKSLLHSNGKTYLNWYQQK | CL0020307-1 VL |
| | PGQPPKLLIYWMSTRASGVPDRFSGSGSGTDFTLTISSLQAEDVA | |
| | VYYCQQFLEFPFTFGQGTKVEIK | |
| 69 | FPGESES | TREM2 epitope |
| 70 | GEKGPCQRV | TREM2 epitope |
| 71 | TLRNLQPHDAGL | TREM2 epitope |
| 72 | VEVL | TREM2 epitope |
| 73 | DIQMTQSPSSVSASVGDRVTITCRASQGISNWLAWYQQKPGKAP | Reference antibody #1 VL |
| | KLLIYAASSLQVGVPLRFSGSGSGTDFTLTISSLQPEDFATYYCQ | |
| | QADSFPRNFGQGTKLEIK | |
| 74 | EVQLVQSGAEVKKPGESLKISCKGSGHSFTNYWIAWVRQMPGK | Reference antibody #1 V_{H} |
| | GLEWMGIIYPGDSDTRYSPSFQGQVTISADKSISTAYLQWSSLKA | |
| | SDTAVYFCARQRTFYYDSSGYFDYWGQGTLVTVSS | |
| 75 | DIQMTQSPSSVSASVGDRVTITCRASQGISSWLAWYQQKPGKAP | Reference antibody #2 VL |
| | KLLIYAASSLQNGVPSRFSGSGSGTDFTLTISSLQPEDFATYFCQQ | |
| | ADSFPRTFGQGTKLEIK | |
| 76 | EVQLVQSGAEVKKPGESLKISCKGSGYSFTSYWIAWVRQMPGK | Reference antibody #2 V_{H} |
| | GLEWMGIIYPGDSDTRYSPSFQGQVTISADKSISTAYLQWSSLKA | |
| | SDTAMYFCARQRTFYYDSSDYFDYWGQGTLVTVSS | |
| 77 | DVVMTQSPDSLAVSLGERATINCRSSQSLVHSNRYTYLHWYQQ | Reference antibody #3 VL |
| | KPGQSPKLLIYKVSNRFSGVPDRFSGSGSGTDFTLKISRVEAEDV | |
| | GVYYCSQSTRVPYTFGQGTKLEIK | |
| 78 | QVQLVQSGAEVKKPGASVKVSCKASGYAFSSQWMNWVRQAP | Reference antibody #3 V_{H} |
| | GQRLEWIGRIYPGGGDTNYAGKFQGRVTITADTSASTAYMELSS | |
| | LRSEDTAVYYCARLLRNQPGESYAMDYWGQGTLVTVSS | |
| 79 | EVQLVESGGGLVQPGGSLRLSCAGSGFTFTDFYMSWVRQAPGK | CL0020188-4 V_{H} |
| | GPEWLSVIRNKANGYTAGYNPSVKGRFTISRDNSKNTLYLQMN | |
| | SLRAEDTAVYYCARLTYGFDYWGQGTLVTVSS | |
| 80 | NGDS | Table 5 hV_{H} |
| 81 | QGDS | Table 5 hV_{H} |
| 82 | NGES | Table 5 hV_{H} |
| 83 | QGES | Table 5 hV_{H} |

What is claimed is:

1. An isolated antibody or antigen-binding fragment thereof that specifically binds to a human TREM2, wherein the antibody or antigen-binding fragment thereof comprises:
(a) a CDR-H1 sequence comprising the sequence of G-F-T-F-T-α₆-F-Y-M-S(SEQ ID NO:48), wherein α₆ is D or N;
(b) a CDR-H2 sequence comprising the sequence of V-I-R-N-β₅-β₆—N-β₈—Y-T-N-β₁₁-β₁₂-Y-N-P-S-V-K-G (SEQ ID NO:49), wherein β₅ is K or R; β₆ is A or P; β₈ is G or A; β₁₁ is A or T; and β₁₂ is G or D;
(c) a CDR-H3 sequence comprising the sequence of γ₁—R-L-γ₄—Y-G-F-D-Y (SEQ ID NO:50), wherein γ₁ is A or T; and γ₄ is T or S;
(d) a CDR-L1 sequence comprising the sequence of Q-S-S-K-S-L-L-H-S-δ₁₀-G-K-T-Y-L-N(SEQ ID NO:51), wherein δ₁₀ is N or T;
(e) a CDR-L2 sequence comprising the sequence of WMSTRAS (SEQ ID NO:8); and
(f) a CDR-L3 sequence comprising the sequence of Q-Q-F-L-E-φ₆-P-F-T (SEQ ID NO:52), wherein φ₆ is Y or F.

2. The isolated antibody or antigen-binding fragment of claim 1, wherein the CDR-H1 sequence is selected from any one of SEQ ID NOS:4 and 12.

3. The isolated antibody or antigen-binding fragment of claim 1 or 2, wherein the CDR-H2 sequence is selected from any one of SEQ ID NOS:5, 13, and 25.

4. The isolated antibody or antigen-binding fragment of any one of claims 1 to 3, wherein the CDR-H3 sequence is selected from any one of SEQ ID NOS:6, 14, and 17.

5. The isolated antibody or antigen-binding fragment of any one of claims 1 to 4, wherein the CDR-L1 sequence is selected from any one of SEQ ID NOS:7 and 23.

6. The isolated antibody or antigen-binding fragment of any one of claims 1 to 5, wherein the CDR-L3 sequence is selected from any one of SEQ ID NOS:9 and 18.

7. The isolated antibody or antigen-binding fragment of any one of claims 1 to 6, wherein the antibody or antigen-binding fragment comprises:
(a) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO: 17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO: 18; or
(b) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO: 17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO: 18; or
(c) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO: 17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO: 18; or
(d) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO: 17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO: 18; or
(e) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:6, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(f) a CDR-H1 comprising the amino acid sequence of SEQ ID NO: 12, a CDR-H2 comprising the amino acid sequence of SEQ ID NO: 13, a CDR-H3 comprising the amino acid sequence of SEQ ID NO: 14, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(g) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO: 17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9.

8. The isolated antibody or antigen-binding fragment of any one of claims 1 to 7, comprising a V_{H} sequence that has at least 85% sequence identity to any one of SEQ ID NOS:2, 10, 15, 19, 21, 24, 26, and 79.

9. The isolated antibody or antigen-binding fragment of claim 8, wherein the V_{H} sequence has at least 90% sequence identity to SEQ ID NO: 15.

10. The isolated antibody or antigen-binding fragment of claim 9, wherein the V_{H} sequence has at least 95% sequence identity to SEQ ID NO: 15.

11. The isolated antibody or antigen-binding fragment of claim 10, wherein the V_{H} sequence comprises SEQ ID NO: 15.

12. The isolated antibody or antigen-binding fragment of claim 8, wherein the V_{H} sequence has at least 90% sequence identity to SEQ ID NO:24.

13. The isolated antibody or antigen-binding fragment of claim 12, wherein the V_{H} sequence has at least 95% sequence identity to SEQ ID NO:24.

14. The isolated antibody or antigen-binding fragment of claim 13, wherein the V_{H} sequence comprises SEQ ID NO:24.

15. The isolated antibody or antigen-binding fragment of claim 8, wherein the V_{H} sequence has at least 90% sequence identity to SEQ ID NO:79.

16. The isolated antibody or antigen-binding fragment of claim 15, wherein the V_{H} sequence has at least 95% sequence identity to SEQ ID NO:79.

17. The isolated antibody or antigen-binding fragment of claim 16, wherein the V_{H} sequence comprises SEQ ID NO:79.

18. The isolated antibody or antigen-binding fragment of any one of claims 1 to 17, comprising a V_{L} sequence that has at least 85% sequence identity to any one of SEQ ID NOS:3, 11, 16, 20, 22, and 68.

19. The isolated antibody or antigen-binding fragment of claim 18, wherein the VL sequence has at least 90% sequence identity to SEQ ID NO: 16.

20. The isolated antibody or antigen-binding fragment of claim 19, wherein the VL sequence has at least 95% sequence identity to SEQ ID NO: 16.

21. The isolated antibody or antigen-binding fragment of claim 20, wherein the VL sequence comprises SEQ ID NO: 16.

22. The isolated antibody or antigen-binding fragment of claim 18, wherein the V_{L} sequence has at least 90% sequence identity to SEQ ID NO:22.

23. The isolated antibody or antigen-binding fragment of claim 22, wherein the VL sequence has at least 95% sequence identity to SEQ ID NO:22.

24. The isolated antibody or antigen-binding fragment of claim 23, wherein the VL sequence comprises SEQ ID NO:22.

25. The isolated antibody or antigen-binding fragment of claim 18, wherein the V_{L} sequence has at least 90% sequence identity to SEQ ID NO:68.

26. The isolated antibody or antigen-binding fragment of claim 25, wherein the VL sequence has at least 95% sequence identity to SEQ ID NO:68.

27. The isolated antibody or antigen-binding fragment of claim 26, wherein the VL sequence comprises SEQ ID NO:68.

28. The isolated antibody or antigen-binding fragment of any one of claims 1 to 27, wherein the antibody or antigen-binding fragment comprises:
(a) a VH sequence comprising SEQ ID NO: 15 and a VL sequence comprising SEQ ID NO: 16; or
(b) a VH sequence comprising SEQ ID NO: 19 and a VL sequence comprising SEQ ID NO:20; or
(c) a VH sequence comprising SEQ ID NO:21 and a VL sequence comprising SEQ ID NO:20; or
(d) a VH sequence comprising SEQ ID NO: 19 and a VL sequence comprising SEQ ID NO:22; or
(e) a VH sequence comprising SEQ ID NO:79 and a VL sequence comprising SEQ ID NO:22; or
(f) a VH sequence comprising SEQ ID NO:24 and a VL sequence comprising SEQ ID NO:20; or
(g) a VH sequence comprising SEQ ID NO:26 and a VL sequence comprising SEQ ID NO:20; or
(h) a VH sequence comprising SEQ ID NO:24 and a VL sequence comprising SEQ ID NO:22; or
(i) a VH sequence comprising SEQ ID NO:26 and a VL sequence comprising SEQ ID NO:22; or
(j) a VH sequence comprising SEQ ID NO:2 and a VL sequence comprising SEQ ID NO:3; or
(k) a VH sequence comprising SEQ ID NO: 10 and a VL sequence comprising SEQ ID NO: 11; or
(l) a VH sequence comprising SEQ ID NO:24 and a VL sequence comprising SEQ ID NO:68.

29. An isolated antibody or antigen-binding fragment thereof that specifically binds to a human TREM2, wherein the antibody or antigen-binding fragment thereof comprises:
(a) a CDR-H1 sequence comprising the sequence of GFSIEDFYIH (SEQ ID NO:29);
(b) a CDR-H2 sequence comprising the sequence of A-O-R-E-β₆-O-β₈-S-K-Y-A-P-K-F-Q-G (SEQ ID NO:47), wherein β₆ is N or Q and β₈ is D or E;
(c) a CDR-H3 sequence comprising the sequence of HADHGNYGSTMDY (SEQ ID NO:31);
(d) a CDR-L1 sequence comprising the sequence of HASQHINVWLS (SEQ ID NO:32);
(e) a CDR-L2 sequence comprising the sequence of KASNLHT (SEQ ID NO:33); and
(f) a CDR-L3 sequence comprising the sequence of QQGQTYPRT (SEQ ID NO:34).

30. The isolated antibody or antigen-binding fragment of claim 29, wherein the CDR-H2 sequence is selected from SEQ ID NOS:30, 39, 41, and 43.

31. The isolated antibody or antigen-binding fragment of claim 29 or 30, wherein the antibody or antigen-binding fragment comprises:
(a) a CDR-H1 comprising the amino acid sequence of SEQ ID NO:29, a CDR-H2 comprising the amino acid sequence of SEQ ID NO:30, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(b) a CDR-H1 comprising the amino acid sequence of SEQ ID NO:29, a CDR-H2 comprising the amino acid sequence of SEQ ID NO:39, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(c) a CDR-H1 comprising the amino acid sequence of SEQ ID NO:29, a CDR-H2 comprising the amino acid sequence of SEQ ID NO:41, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(d) a CDR-H1 comprising the amino acid sequence of SEQ ID NO:29, a CDR-H2 comprising the amino acid sequence of SEQ ID NO:43, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34.

32. The isolated antibody or antigen-binding fragment of any one of claims 29 to 31, comprising a V_{H} sequence that has at least 85% sequence identity to any one of SEQ ID NOS:27, 35, 37, 38, 40, 42, 44, 45, and 46.

33. The isolated antibody or antigen-binding fragment of claim 32, wherein the V_{H} sequence has at least 90% sequence identity to SEQ ID NO:27.

34. The isolated antibody or antigen-binding fragment of claim 33, wherein the V_{H} sequence has at least 95% sequence identity to SEQ ID NO:27.

35. The isolated antibody or antigen-binding fragment of claim 34, wherein the VH sequence comprises SEQ ID NO:27.

36. The isolated antibody or antigen-binding fragment of any one of claims 29 to 35, comprising a VL sequence that has at least 85% sequence identity to SEQ ID NO:28 or SEQ ID NO:36.

37. The isolated antibody or antigen-binding fragment of claim 36, wherein the V_{L} sequence has at least 90% sequence identity to SEQ ID NO:28.

38. The isolated antibody or antigen-binding fragment of claim 37, wherein the V_{L} sequence has at least 95% sequence identity to SEQ ID NO:28.

39. The isolated antibody or antigen-binding fragment of claim 38, wherein the VL sequence comprises SEQ ID NO:28.

40. The isolated antibody or antigen binding fragment of claim 36, wherein the antibody or antigen-binding fragment comprises:
(a) a VH sequence comprising SEQ ID NO:27 and a VL sequence comprising SEQ ID NO:28; or
(b) a VH sequence comprising SEQ ID NO:35 and a VL sequence comprising SEQ ID NO:36; or
(c) a VH sequence comprising SEQ ID NO:37 and a VL sequence comprising SEQ ID NO:36; or
(d) a VH sequence comprising SEQ ID NO:38 and a VL sequence comprising SEQ ID NO:36; or
(e) a VH sequence comprising SEQ ID NO:40 and a VL sequence comprising SEQ ID NO:36; or
(f) a VH sequence comprising SEQ ID NO:42 and a VL sequence comprising SEQ ID NO:36; or
(g) a VH sequence comprising SEQ ID NO:44 and a VL sequence comprising SEQ ID NO:36; or
(h) a VH sequence comprising SEQ ID NO:45 and a VL sequence comprising SEQ ID NO:36; or
(i) a VH sequence comprising SEQ ID NO:46 and a VL sequence comprising SEQ ID NO:36.

41. An isolated antibody or antigen-binding fragment thereof that specifically binds to a human TREM2, wherein the antibody or antigen-binding fragment thereof comprises:
(a) a CDR-H1 sequence comprising the amino acid sequence of any one of SEQ ID NOS:4, 12, and 29;
(b) a CDR-H2 sequence comprising the amino acid sequence of any one of SEQ ID NOS:5, 13, 25, 30, 39, 41, and 43;
(c) a CDR-H3 sequence comprising the amino acid sequence of any one of SEQ ID NOS:6, 14, 17, and 31;
(d) a CDR-L1 sequence comprising the amino acid sequence of any one of SEQ ID NOS:7, 23, and 32;
(e) a CDR-L2 sequence comprising the amino acid sequence of any one of SEQ ID NOS:8 and 33; and
(f) a CDR-L3 sequence comprising the amino acid sequence of any one of SEQ ID NOS:9, 18, and 34.

42. The isolated antibody or antigen-binding fragment of claim 41, wherein the antibody or antigen-binding fragment comprises:
(a) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:6, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(b) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO: 17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO: 18; or
(c) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO: 17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO: 18; or
(d) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO: 17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO: 18; or
(e) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO: 17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO: 18; or
(f) a CDR-H1 comprising the amino acid sequence of SEQ ID NO: 12, a CDR-H2 comprising the amino acid sequence of SEQ ID NO: 13, a CDR-H3 comprising the amino acid sequence of SEQ ID NO: 14, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(g) a CDR-H1 comprising the amino acid sequence of SEQ ID NO:29, a CDR-H2 comprising the amino acid sequence of SEQ ID NO:30, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(h) a CDR-H1 comprising the amino acid sequence of SEQ ID NO:29, a CDR-H2 comprising the amino acid sequence of SEQ ID NO:39, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(i) a CDR-H1 comprising the amino acid sequence of SEQ ID NO:29, a CDR-H2 comprising the amino acid sequence of SEQ ID NO:41, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(j) a CDR-H1 comprising the amino acid sequence of SEQ ID NO:29, a CDR-H2 comprising the amino acid sequence of SEQ ID NO:43, a CDR-H3 comprising the amino acid sequence of SEQ ID NO:31, a CDR-L1 comprising the amino acid sequence of SEQ ID NO:32, a CDR-L2 comprising the amino acid sequence of SEQ ID NO:33, and a CDR-L3 comprising the amino acid sequence of SEQ ID NO:34; or
(k) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO: 17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9.

43. The isolated antibody or antigen-binding fragment of claim 41 or 42, comprising a heavy chain variable region that has at least 85% sequence identity to any one of SEQ ID NOS:2, 10, 15, 19, 21, 24, 26, 27, 35, 37, 38, 40, 42, 44, 45, 46, and 79.

44. The isolated antibody or antigen-binding fragment of any one of claims 41 to 43, comprising a light chain variable region that has at least 85% sequence identity to any one of SEQ ID NOS:3, 11, 16, 20, 22, 28, and 36.

45. The isolated antibody or antigen-binding fragment of any one of claims 41 to 44, wherein the antibody or antigen-binding fragment comprises:
(a) a VH sequence that has at least 85% sequence identity to SEQ ID NO:2 and a VL sequence that has at least 85% sequence identity to SEQ ID NO:3; or
(b) a VH sequence that has at least 85% sequence identity to SEQ ID NO: 10 and a VL sequence that has at least 85% sequence identity to SEQ ID NO: 11; or
(c) a VH sequence that has at least 85% sequence identity to SEQ ID NO: 15 and a VL sequence that has at least 85% sequence identity to SEQ ID NO: 16; or
(d) a VH sequence that has at least 85% sequence identity to SEQ ID NO: 19 and a VL sequence that has at least 85% sequence identity to SEQ ID NO:20; or
(e) a VH sequence that has at least 85% sequence identity to SEQ ID NO:21 and a VL sequence that has at least 85% sequence identity to SEQ ID NO:20; or
(f) a VH sequence that has at least 85% sequence identity to SEQ ID NO: 19 and a VL sequence that has at least 85% sequence identity to SEQ ID NO:22; or
(g) a VH sequence that has at least 85% sequence identity to SEQ ID NO:79 and a VL sequence that has at least 85% sequence identity to SEQ ID NO:22; or
(h) a VH sequence that has at least 85% sequence identity to SEQ ID NO:24 and a VL sequence that has at least 85% sequence identity to SEQ ID NO:20; or
(i) a VH sequence that has at least 85% sequence identity to SEQ ID NO:26 and a VL sequence that has at least 85% sequence identity to SEQ ID NO:20; or
(j) a VH sequence that has at least 85% sequence identity to SEQ ID NO:24 and a VL sequence that has at least 85% sequence identity to SEQ ID NO:22; or
(k) a VH sequence that has at least 85% sequence identity to SEQ ID NO:26 and a VL sequence that has at least 85% sequence identity to SEQ ID NO:22; or
(l) a VH sequence that has at least 85% sequence identity to SEQ ID NO:27 and a VL sequence that has at least 85% sequence identity to SEQ ID NO:28; or
(m) a VH sequence that has at least 85% sequence identity to SEQ ID NO:35 and a VL sequence that has at least 85% sequence identity to SEQ ID NO:36; or
(n) a VH sequence that has at least 85% sequence identity to SEQ ID NO:37 and a VL sequence that has at least 85% sequence identity to SEQ ID NO:36; or
(o) a VH sequence that has at least 85% sequence identity to SEQ ID NO:38 and a VL sequence that has at least 85% sequence identity to SEQ ID NO:36; or
(p) a VH sequence that has at least 85% sequence identity to SEQ ID NO:40 and a VL sequence that has at least 85% sequence identity to SEQ ID NO:36; or
(q) a VH sequence that has at least 85% sequence identity to SEQ ID NO:42 and a VL sequence that has at least 85% sequence identity to SEQ ID NO:36; or
(r) a VH sequence that has at least 85% sequence identity to SEQ ID NO:44 and a VL sequence that has at least 85% sequence identity to SEQ ID NO:36; or
(s) a VH sequence that has at least 85% sequence identity to SEQ ID NO:45 and a VL sequence that has at least 85% sequence identity to SEQ ID NO:36; or
(t) a VH sequence that has at least 85% sequence identity to SEQ ID NO:46 and a VL sequence that has at least 85% sequence identity to SEQ ID NO:36; or
(u) a VH sequence that has at least 85% sequence identity to SEQ ID NO:24 and a VL sequence that has at least 85% sequence identity to SEQ ID NO:68.

46. An isolated antibody or antigen-binding fragment thereof that specifically binds to a human triggering receptor expressed on myeloid cells 2 (TREM2), wherein the antibody or antigen-binding fragment thereof recognizes an epitope that is the same or substantially the same as the epitope recognized by antibody clone selected from the group consisting of: Clone CL0020306, Clone CL0020188, Clone CL0020188-1, Clone CL0020188-2, Clone CL0020188-3, Clone CL0020188-4, Clone CL0020188-5, Clone CL0020188-6, Clone CL0020188-7, Clone CL0020188-8, Clone CL0020307, Clone CL0020123, Clone CL0020123-1, Clone CL0020123-2, Clone CL0020123-3, Clone CL0020123-4, Clone CL0020123-5, Clone CL0020123-6, Clone CL0020123-7, and Clone CL0020123-8.

47. The isolated antibody or antigen-binding fragment of claim 46, wherein the antibody or antigen-binding fragment recognizes an epitope that is the same or substantially the same as the epitope recognized by an antibody clone selected from the group consisting of: Clone CL0020123, Clone CL0020123-1, Clone CL0020123-2, Clone CL0020123-3, Clone CL0020123-4, Clone CL0020123-5, Clone CL0020123-6, Clone CL0020123-7, and Clone CL0020123-8.

48. The isolated antibody or antigen-binding fragment of claim 47, wherein the antibody or antigen-binding fragment recognizes one or more of the following in SEQ ID NO: 1: (i) amino acid residues 55-63 (GEKGPCQRV (SEQ ID NO:70)), (ii) amino acids 96-107 (TLRNLQPHDAGL (SEQ ID NO:71)), and (iii) amino acid residues 126-129 (VEVL (SEQ ID NO: 72)).

49. The isolated antibody or antigen-binding fragment of claim 46, wherein the antibody or antigen-binding fragment recognizes an epitope that is the same or substantially the same as the epitope recognized by an antibody clone selected from the group consisting of: Clone CL0020188, Clone CL0020188-1, Clone CL0020188-2, Clone CL0020188-3, Clone CL0020188-4, Clone CL0020188-5, Clone CL0020188-6, Clone CL0020188-7, Clone CL0020188-8, Clone CL0020307, and Clone CL0020306.

50. The isolated antibody or antigen-binding fragment of claim 49, wherein the antibody or antigen-binding fragment recognizes amino acid residues 143-149 (FPGESES (SEQ ID NO:69)) in SEQ ID NO: 1.

51. An isolated antibody or antigen-binding fragment thereof that specifically binds to a human TREM2, wherein the antibody or antigen-binding fragment thereof recognizes an epitope comprising or consisting of one or more of the following in SEQ ID NO: 1: (i) amino acid residues 55-63 (GEKGPCQRV (SEQ ID NO:70)), (ii) amino acids 96-107 (TLRNLQPHDAGL (SEQ ID NO:71)), and (iii) amino acid residues 126-129 (VEVL (SEQ ID NO: 72)).

52. An isolated antibody or antigen-binding fragment thereof that specifically binds to a human TREM2, wherein the antibody or antigen-binding fragment thereof recognizes an epitope comprising or consisting of amino acid residues 143-149 (FPGESES (SEQ ID NO:69)) in SEQ ID NO: 1.

53. The isolated antibody or antigen-binding fragment thereof of any one of claims 1 to 52, wherein the antibody or antigen-binding fragment thereof decreases levels of soluble TREM2 protein (sTREM2).

54. The isolated antibody or antigen-binding fragment thereof of any one of claims 1 to 53, wherein the antibody or antigen-binding fragment thereof enhances TREM2 activity.

55. The isolated antibody or antigen-binding fragment thereof of claim 54, wherein the antibody or antigen-binding fragment thereof enhances phagocytosis or enhances the migration, differentiation, function, or survival of myeloid cells, microglia, or macrophages.

56. The isolated antibody or antigen-binding fragment thereof of claim 55, wherein the antibody or antigen-binding fragment thereof enhances microglia function without increasing neuroinflammation.

57. The isolated antibody or antigen-binding fragment thereof of claim 54, wherein the antibody or antigen-binding fragment thereof enhances Syk phosphorylation.

58. The isolated antibody or antigen-binding fragment thereof of claim 57, wherein the antibody or antigen-binding fragment thereof enhances Syk phosphorylation in the presence of a TREM2 ligand.

59. The isolated antibody or antigen-binding fragment thereof of any one of claims 1 to 58, wherein the antibody or antigen-binding fragment thereof exhibits cross reactivity with a cynomolgus TREM2 protein.

60. The isolated antibody or antigen-binding fragment thereof of any one of claims 1 to 59, wherein the antibody is a monoclonal antibody.

61. The isolated antibody or antigen-binding fragment thereof of any one of claims 1 to 59, wherein the antibody is a chimeric antibody.

62. The isolated antibody or antigen-binding fragment thereof of any one of claims 1 to 59, wherein the antibody is a humanized antibody.

63. The isolated antibody or antigen-binding fragment thereof of any one of claims 1 to 59, wherein the antibody is a fully human antibody.

64. The isolated antibody or antigen-binding fragment thereof of any one of claims 1 to 59, wherein the antigen-binding fragment is a Fab, a F(ab′)2, a scFv, or a bivalent scFv.

65. A pharmaceutical composition comprising the isolated antibody or antigen-binding fragment thereof of any one of claims 1 to 64 and a pharmaceutically acceptable carrier.

66. An antibody or antigen-binding fragment thereof that competes with the isolated antibody of any one of claims 1 to 64 for binding to the human TREM2 protein.

67. A kit comprising:
the isolated antibody or antigen-binding fragment thereof of any one of claims 1 to 64 or the pharmaceutical composition of claim 65; and
instructions for use thereof.

68. A method of treating a neurodegenerative disease in a subject, comprising administering to the subject the isolated antibody or antigen-binding fragment thereof of any one of claims 1 to 64 or the pharmaceutical composition of claim 65.

69. The method of claim 68, wherein the neurodegenerative disease is selected from the group consisting of: Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing pan encephalitis, and tangle only dementia.

70. A method of decreasing levels of sTREM2 in a subject having a neurodegenerative disease, comprising administering to the subject the isolated antibody or antigen-binding fragment thereof of any one of claims 1 to 64 or the pharmaceutical composition of claim 65.

71. A method of enhancing TREM2 activity in a subject having a neurodegenerative disease, comprising administering to the subject the isolated antibody or antigen-binding fragment thereof of any one of claims 1 to 64 or the pharmaceutical composition of claim 65.


SEQUENCE LISTING (83 sequences)

SEQ ID NO: 1 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - protein, 118 aa, synthetic construct
  REGION 1..118: note = "Description of Artificial Sequence: Synthetic polypeptide"
EVKLLDSGGGLVQAGGSLRLSCAGSGFTFTDFYMSWIRQPPGKAPEWLGVIRNKANGYTAGYNPSVKGRFTISRDNTQNILYLQMNTLRAEDTAIYYCARLSYGFDYWGQGVMVTVSS

SEQ ID NO: 3 - protein, 112 aa, synthetic construct
  REGION 1..112: note = "Description of Artificial Sequence: Synthetic polypeptide"
DIVMTQGALPNPVPSGESASITCQSSKSLLHSNGKTYLNWYLQRPGQSPQLLIYWMSTRASGVSDRFSGSGSGTDFTLKISSVEAEDVGVYYCQQFLEFPFTFGSGTKLEIK

SEQ ID NO: 4 - protein, 10 aa, synthetic construct
  REGION 1..10: note = Description of Artificial Sequence: Synthetic peptide"
GFTFTDFYMS

SEQ ID NO: 5 - protein, 19 aa, synthetic construct
  REGION 1..19: note = Description of Artificial Sequence: Synthetic peptide"
VIRNKANGYTAGYNPSVKG

SEQ ID NO: 6 - protein, 9 aa, synthetic construct
  REGION 1..9: note = "Description of Artificial Sequence: Synthetic peptide"
ARLSYGFDY

SEQ ID NO: 7 - protein, 16 aa, synthetic construct
  REGION 1..16: note = "Description of Artificial Sequence: Synthetic peptide"
QSSKSLLHSNGKTYLN

SEQ ID NO: 8 - protein, 7 aa, synthetic construct
  REGION 1..7: note = "Description of Artificial Sequence: Synthetic peptide"
WMSTRAS

SEQ ID NO: 9 - protein, 9 aa, synthetic construct
  REGION 1..9: note = "Description of Artificial Sequence: Synthetic peptide"
QQFLEFPFT

SEQ ID NO: 10 - protein, 118 aa, synthetic construct
  REGION 1..118: note = "Description of Artificial Sequence: Synthetic polypeptide"
EVKLLESGGGLVQPGGSLRLSCAASGFTFTNFYMSWIRQPPGRAPEWLGVIRNRPNGYTTDYNPSVKGRFTISRDNTQNILYLQMSTLRADDTAFYYCTRLTYGFDYWGQGVMVTVSS

SEQ ID NO: 11 - protein, 112 aa, synthetic construct
  REGION 1..112: note = "Description of Artificial Sequence: Synthetic polypeptide"
DIVMTQGALPNPVPSGESASITCQSSKSLLHSNGKTYLNWYLQRPGQSPQLLIYWMSTRASGVSDRFSGSGSGTDFTLKISSVEAEVVGVYYCQQFLEFPFTFGSGTKLEIK

SEQ ID NO: 12 - protein, 10 aa, synthetic construct
  REGION 1..10: note = "Description of Artificial Sequence: Synthetic peptide"
GFTFTNFYMS

SEQ ID NO: 13 - protein, 19 aa, synthetic construct
  REGION 1..19: note = "Description of Artificial Sequence: Synthetic peptide"
VIRNRPNGYTTDYNPSVKG

SEQ ID NO: 14 - protein, 9 aa, synthetic construct
  REGION 1..9: note = "Description of Artificial Sequence: Synthetic peptide"
TRLTYGFDY

SEQ ID NO: 15 - protein, 118 aa, synthetic construct
  REGION 1..118: note = "Description of Artificial Sequence: Synthetic polypeptide"
EVKLLDSGGGLVQAGGSLRLSCAGSGFTFTDFYMSWIRQPPGKAPEWLGVIRNKANGYTAGYNPSVKGRFTISRDNTQNILYLQMNTLRAEDTAIYYCARLTYGFDYWGQGVMVTVSS

SEQ ID NO: 16 - protein, 112 aa, synthetic construct
  REGION 1..112: note = "Description of Artificial Sequence: Synthetic polypeptide"
DIVMTQGALPNPVPSGESASITCQSSKSLLHSNGKTYLNWYLQRPGQSPQLLIYWMSTRASGVSDRFSGSGSGTDFTLKISSVEAEDVGVYYCQQFLEYPFTFGSGTKLEIK

SEQ ID NO: 17 - protein, 9 aa, synthetic construct
  REGION 1..9: note = "Description of Artificial Sequence: Synthetic peptide"
ARLTYGFDY

SEQ ID NO: 18 - protein, 9 aa, synthetic construct
  REGION 1..9: note = "Description of Artificial Sequence: Synthetic peptide"
QQFLEYPFT

SEQ ID NO: 19 - protein, 118 aa, synthetic construct
  REGION 1..118: note = "Description of Artificial Sequence: Synthetic polypeptide"
EVQLVESGGGLVQPGGSLRLSCAASGFTFTDFYMSWVRQAPGKGLEWVSVIRNKANGYTAGYNPSVKGRFTISRDNSKNTLYLQMNSLRAEDTAVYYCARLTYGFDYWGQGTLVTVSS

SEQ ID NO: 20 - protein, 112 aa, synthetic construct
  REGION 1..112: note = "Description of Artificial Sequence: Synthetic polypeptide"
DIVMTQTPLSLPVTPGEPASISCQSSKSLLHSNGKTYLNWYLQKPGQSPQLLIYWMSTRASGVPDRFSGSGSGTDFTLKISRVEAEDVGVYYCQQFLEYPFTFGQGTKVEIK

SEQ ID NO: 21 - protein, 118 aa, synthetic construct
  REGION 1..118: note = "Description of Artificial Sequence: Synthetic polypeptide"
EVQLVESGGGLVQPGGSLRLSCAGSGFTFTDFYMSWVRQAPGKGLEWVSVIRNKANGYTAGYNPSVKGRFTISRDNSKNTLYLQMNSLRAEDTAVYYCARLTYGFDYWGQGTLVTVSS

SEQ ID NO: 22 - protein, 112 aa, synthetic construct
  REGION 1..112: note = "Description of Artificial Sequence: Synthetic polypeptide"
DIVMTQTPLSLPVTPGEPASISCQSSKSLLHSTGKTYLNWYLQKPGQSPQLLIYWMSTRASGVPDRFSGSGSGTDFTLKISRVEAEDVGVYYCQQFLEYPFTFGQGTKVEIK

SEQ ID NO: 23 - protein, 16 aa, synthetic construct
  REGION 1..16: note = "Description of Artificial Sequence: Synthetic peptide"
QSSKSLLHSTGKTYLN

SEQ ID NO: 24 - protein, 118 aa, synthetic construct
  REGION 1..118: note = "Description of Artificial Sequence: Synthetic polypeptide"
EVQLVESGGGLVQPGGSLRLSCAASGFTFTDFYMSWVRQAPGKGLEWVSVIRNKANAYTAGYNPSVKGRFTISRDNSKNTLYLQMNSLRAEDTAVYYCARLTYGFDYWGQGTLVTVSS

SEQ ID NO: 25 - protein, 19 aa, synthetic construct
  REGION 1..19: note = "Description of Artificial Sequence: Synthetic peptide"
VIRNKANAYTAGYNPSVKG

SEQ ID NO: 26 - protein, 118 aa, synthetic construct
  REGION 1..118: note = "Description of Artificial Sequence: Synthetic polypeptide"
EVQLVESGGGLVQPGGSLRLSCAGSGFTFTDFYMSWVRQAPGKGPEWLSVIRNKANAYTAGYNPSVKGRFTISRDNSKNTLYLQMNSLRAEDTAVYYCARLTYGFDYWGQGTLVTVSS

SEQ ID NO: 27 - protein, 120 aa, synthetic construct
  REGION 1..120: note = "Description of Artificial Sequence: Synthetic polypeptide"
EVQLQQSGAELVRSGASVKLSCTASGFSIEDFYIHWVKQRPEQGLEWIGWIDPENGDSKYAPKFQGKATMTADTSSNTAYLHLSSLTSEDTAVYYCHADHGNYGSTMDYWGQGTSVTVSS

SEQ ID NO: 28 - protein, 107 aa, synthetic construct
  REGION 1..107: note = "Description of Artificial Sequence: Synthetic polypeptide"
DIQMNQSPSSLSASLGDTVTITCHASQHINVWLSWYQQKPGDHPKLLIYKASNLHTGVPSRFSGSGSGTGFTLTISSLQPEDIATYYCQQGQTYPRTFGGGTKLEIK

SEQ ID NO: 29 - protein, 10 aa, synthetic construct
  REGION 1..10: note = "Description of Artificial Sequence: Synthetic peptide"
GFSIEDFYIH

SEQ ID NO: 30 - protein, 17 aa, synthetic construct
  REGION 1..17: note = "Description of Artificial Sequence: Synthetic peptide"
WIDPENGDSKYAPKFQG

SEQ ID NO: 31 - protein, 13 aa, synthetic construct
  REGION 1..13: note = "Description of Artificial Sequence: Synthetic peptide"
HADHGNYGSTMDY

SEQ ID NO: 32 - protein, 11 aa, synthetic construct
  REGION 1..11: note = "Description of Artificial Sequence: Synthetic peptide"
HASQHINVWLS

SEQ ID NO: 33 - protein, 7 aa, synthetic construct
  REGION 1..7: note = "Description of Artificial Sequence: Synthetic peptide"
KASNLHT

SEQ ID NO: 34 - protein, 9 aa, synthetic construct
  REGION 1..9: note = "Description of Artificial Sequence: Synthetic peptide"
QQGQTYPRT

SEQ ID NO: 35 - protein, 120 aa, synthetic construct
  REGION 1..120: note = "Description of Artificial Sequence: Synthetic polypeptide"
QVQLVQSGAEVKKPGASVKVSCKASGFSIEDFYIHWVRQAPGQGLEWMGWIDPENGDSKYAPKFQGRATITADTSTSTAYMELSSLRSEDTAVYYCHADHGNYGSTMDYWGQGTLVTVSS

SEQ ID NO: 36 - protein, 107 aa, synthetic construct
  REGION 1..107: note = "Description of Artificial Sequence: Synthetic polypeptide"
DIQMTQSPSSLSASVGDRVTITCHASQHINVWLSWYQQKPGKAPKLLIYKASNLHTGVPSRFSGSGSGTDFTLTISSLQPEDFATYYCQQGQTYPRTFGQGTKVEIK

SEQ ID NO: 37 - protein, 120 aa, synthetic construct
  REGION 1..120: note = "Description of Artificial Sequence: Synthetic polypeptide"
QVQLVQSGAEVKKPGASVKVSCKASGFSIEDFYIHWVRQAPGQGLEWMGWIDPENGDSKYAPKFQGRVTITADTSTSTAYMELSSLRSEDTAVYYCHADHGNYGSTMDYWGQGTLVTVSS

SEQ ID NO: 38 - protein, 120 aa, synthetic construct
  REGION 1..120: note = "Description of Artificial Sequence: Synthetic polypeptide"
QVQLVQSGAEVKKPGASVKVSCKASGFSIEDFYIHWVRQAPGQGLEWMGWIDPEQGDSKYAPKFQGRATITADTSTSTAYMELSSLRSEDTAVYYCHADHGNYGSTMDYWGQGTLVTVSS

SEQ ID NO: 39 - protein, 17 aa, synthetic construct
  REGION 1..17: note = "Description of Artificial Sequence: Synthetic peptide"
WIDPEQGDSKYAPKFQG

SEQ ID NO: 40 - protein, 120 aa, synthetic construct
  REGION 1..120: note = "Description of Artificial Sequence: Synthetic polypeptide"
QVQLVQSGAEVKKPGASVKVSCKASGFSIEDFYIHWVRQAPGQGLEWMGWIDPENGESKYAPKFQGRATITADTSTSTAYMELSSLRSEDTAVYYCHADHGNYGSTMDYWGQGTLVTVSS

SEQ ID NO: 41 - protein, 17 aa, synthetic construct
  REGION 1..17: note = "Description of Artificial Sequence: Synthetic peptide"
WIDPENGESKYAPKFQG

SEQ ID NO: 42 - protein, 120 aa, synthetic construct
  REGION 1..120: note = "Description of Artificial Sequence: Synthetic polypeptide"
QVQLVQSGAEVKKPGASVKVSCKASGFSIEDFYIHWVRQAPGQGLEWMGWIDPEQGESKYAPKFQGRATITADTSTSTAYMELSSLRSEDTAVYYCHADHGNYGSTMDYWGQGTLVTVSS

SEQ ID NO: 43 - protein, 17 aa, synthetic construct
  REGION 1..17: note = "Description of Artificial Sequence: Synthetic peptide"
WIDPEQGESKYAPKFQG

SEQ ID NO: 44 - protein, 120 aa, synthetic construct
  REGION 1..120: note = "Description of Artificial Sequence: Synthetic polypeptide"
QVQLVQSGAEVKKPGASVKVSCKASGFSIEDFYIHWVRQAPGQGLEWMGWIDPEQGDSKYAPKFQGRVTITADTSTSTAYMELSSLRSEDTAVYYCHADHGNYGSTMDYWGQGTLVTVSS

SEQ ID NO: 45 - protein, 120 aa, synthetic construct
  REGION 1..120: note = "Description of Artificial Sequence: Synthetic polypeptide"
QVQLVQSGAEVKKPGASVKVSCKASGFSIEDFYIHWVRQAPGQGLEWMGWIDPENGESKYAPKFQGRVTITADTSTSTAYMELSSLRSEDTAVYYCHADHGNYGSTMDYWGQGTLVTVSS

SEQ ID NO: 46 - protein, 120 aa, synthetic construct
  REGION 1..120: note = "Description of Artificial Sequence: Synthetic polypeptide"
QVQLVQSGAEVKKPGASVKVSCKASGFSIEDFYIHWVRQAPGQGLEWMGWIDPEQGESKYAPKFQGRVTITADTSTSTAYMELSSLRSEDTAVYYCHADHGNYGSTMDYWGQGTLVTVSS

SEQ ID NO: 47 - protein, 17 aa, synthetic construct
  REGION 1..17: note = "Description of Artificial Sequence: Synthetic peptide"
  VARIANT 6: note = X can be N or Q
  VARIANT 8: note = X can be D or E
WIDPEXGXSKYAPKFQG

SEQ ID NO: 48 - protein, 10 aa, synthetic construct
  REGION 1..10: note = "Description of Artificial Sequence: Synthetic peptide"
GFTFTBFYMS

SEQ ID NO: 49 - protein, 19 aa, synthetic construct
  REGION 1..19: note = "Description of Artificial Sequence: Synthetic peptide"
  VARIANT 5: note = X can be K or R
  VARIANT 6: note = X can be A or P
  VARIANT 8: note = X can be G or A
  VARIANT 11: note = X can be A or T
  VARIANT 12: note = X can be G or D
VIRNXXNXYTXXYNPSVKG

SEQ ID NO: 50 - protein, 9 aa, synthetic construct
  REGION 1..9: note = "Description of Artificial Sequence: Synthetic peptide"
  VARIANT 1: note = X can be A or T
  VARIANT 4: note = X can be T or S
XRLXYGFDY

SEQ ID NO: 51 - protein, 16 aa, synthetic construct
  REGION 1..16: note = "Description of Artificial Sequence: Synthetic peptide"
  VARIANT 10: note = X can be N or T
QSSKSLLHSXGKTYLN

SEQ ID NO: 52 - protein, 9 aa, synthetic construct
  REGION 1..9: note = "Description of Artificial Sequence: Synthetic peptide"
  VARIANT 6: note = X can be Y or F
QQFLEXPFT

SEQ ID NO: 53 - DNA, 21 bp, synthetic construct
  misc_feature 1..21: note = "Description of Artificial Sequence: Synthetic primer"
  source 1..21: mol_type = other DNA
aggaatgtggggagcacggag

SEQ ID NO: 54 - DNA, 24 bp, synthetic construct
  misc_feature 1..24: note = "Description of Artificial Sequence: Synthetic primer"
  source 1..24: mol_type = other DNA
tgcatcgcattgtctgagtaggtg

SEQ ID NO: 55 - DNA, 22 bp, synthetic construct
  misc_feature 1..22: note = "Description of Artificial Sequence: Synthetic primer"
  source 1..22: mol_type = other DNA
accctagtcctgactgttgctc

SEQ ID NO: 56 - DNA, 25 bp, synthetic construct
  misc_feature 1..25: note = "Description of Artificial Sequence: Synthetic primer"
  source 1..25: mol_type = other DNA
tataggaacttcgcgacacggacac

SEQ ID NO: 57 - DNA, 21 bp, synthetic construct
  misc_feature 1..21: note = "Description of Artificial Sequence: Synthetic primer"
  source 1..21: mol_type = other DNA
acaggagggacctaccttcag

SEQ ID NO: 58 - DNA, 24 bp, synthetic construct
  misc_feature 1..24: note = "Description of Artificial Sequence: Synthetic primer"
  source 1..24: mol_type = other DNA
gcctgcctttcagagacctcagtc

SEQ ID NO: 59 - DNA, 24 bp, synthetic construct
  misc_feature 1..24: note = "Description of Artificial Sequence: Synthetic primer"
  source 1..24: mol_type = other DNA
cctctccggctgctcatcttactc

SEQ ID NO: 60 - DNA, 24 bp, synthetic construct
  misc_feature 1..24: note = "Description of Artificial Sequence: Synthetic primer"
  source 1..24: mol_type = other DNA
gtctctcagccctggcagagtttg

SEQ ID NO: 61 - DNA, 24 bp, synthetic construct
  misc_feature 1..24: note = "Description of Artificial Sequence: Synthetic primer"
  source 1..24: mol_type = other DNA
cgcctaccctagtcctgactgttg

SEQ ID NO: 62 - DNA, 23 bp, synthetic construct
  misc_feature 1..23: note = "Description of Artificial Sequence: Synthetic primer"
  source 1..23: mol_type = other DNA
aaagcctacagcatcctcacctc

SEQ ID NO: 63 - DNA, 23 bp, synthetic construct
  misc_feature 1..23: note = "Description of Artificial Sequence: Synthetic primer"
  source 1..23: mol_type = other DNA
gcatcatggggttgtagattccg

SEQ ID NO: 64 - protein, 5 aa, synthetic construct
  REGION 1..5: note = "Description of Artificial Sequence: Synthetic peptide"
GGGGS

SEQ ID NO: 65 - protein, 6 aa, synthetic construct
  REGION 1..6: note = "Description of Artificial Sequence: Synthetic 6xHis tag"
HHHHHH

SEQ ID NO: 66 - protein, 140 aa, synthetic construct
  REGION 1..140: note = "Description of Artificial Sequence: Synthetic polypeptide"
QVQLQQPGAELVKPGASVKLSCKASGYTFTSYWMHWVKQSPGRGLEWIGRSDPTTGGTNYNEKFKTKATLTVDKPSSTAYMQLSSLTSDDSAVYYCVRTSGTGDYWGQGTSLTVSSAKTTAPSVYPLAPVCGGTTGSSVT

SEQ ID NO: 67 - protein, 140 aa, synthetic construct
  REGION 1..140: note = "Description of Artificial Sequence: Synthetic polypeptide"
DVVMTQTPLSLPVSLGDQASISCRSSQSLVHNNGNTFLHWYLQKPGQSPKLLIYKVSNRFSGVPDRFSGSGSGTDFTLKISRVEAEDLGVYFCSQTTHVPPTFGGGTKLEIKRADAAPTVSIFPPSSEQLTSGGASVVCF

SEQ ID NO: 68 - protein, 112 aa, synthetic construct
  REGION 1..112: note = "Description of Artificial Sequence: Synthetic polypeptide"
DIVMTQSPDSLAVSLGERATINCQSSKSLLHSNGKTYLNWYQQKPGQPPKLLIYWMSTRASGVPDRFSGSGSGTDFTLTISSLQAEDVAVYYCQQFLEFPFTFGQGTKVEIK

SEQ ID NO: 69 - protein, 7 aa, synthetic construct
  REGION 1..7: note = "Description of Artificial Sequence: Synthetic peptide"
FPGESES

SEQ ID NO: 70 - protein, 9 aa, synthetic construct
  REGION 1..9: note = "Description of Artificial Sequence: Synthetic peptide"
GEKGPCQRV

SEQ ID NO: 71 - protein, 12 aa, synthetic construct
  REGION 1..12: note = "Description of Artificial Sequence: Synthetic polypeptide"
TLRNLQPHDAGL

SEQ ID NO: 72 - protein, 4 aa, synthetic construct
  REGION 1..4: note = "Description of Artificial Sequence: Synthetic peptide"
VEVL

SEQ ID NO: 73 - protein, 107 aa, synthetic construct
  REGION 1..107: note = "Description of Artificial Sequence: Synthetic polypeptide"
DIQMTQSPSSVSASVGDRVTITCRASQGISNWLAWYQQKPGKAPKLLIYAASSLQVGVPLRFSGSGSGTDFTLTISSLQPEDFATYYCQQADSFPRNFGQGTKLEIK

SEQ ID NO: 74 - protein, 123 aa, synthetic construct
  REGION 1..123: note = "Description of Artificial Sequence: Synthetic polypeptide"
EVQLVQSGAEVKKPGESLKISCKGSGHSFTNYWIAWVRQMPGKGLEWMGIIYPGDSDTRYSPSFQGQVTISADKSISTAYLQWSSLKASDTAVYFCARQRTFYYDSSGYFDYWGQGTLVTVSS

SEQ ID NO: 75 - protein, 107 aa, synthetic construct
  REGION 1..107: note = "Description of Artificial Sequence: Synthetic polypeptide"
DIQMTQSPSSVSASVGDRVTITCRASQGISSWLAWYQQKPGKAPKLLIYAASSLQNGVPSRFSGSGSGTDFTLTISSLQPEDFATYFCQQADSFPRTFGQGTKLEIK

SEQ ID NO: 76 - protein, 123 aa, synthetic construct
  REGION 1..123: note = "Description of Artificial Sequence: Synthetic polypeptide"
EVQLVQSGAEVKKPGESLKISCKGSGYSFTSYWIAWVRQMPGKGLEWMGIIYPGDSDTRYSPSFQGQVTISADKSISTAYLQWSSLKASDTAMYFCARQRTFYYDSSDYFDYWGQGTLVTVSS

SEQ ID NO: 77 - protein, 112 aa, synthetic construct
  REGION 1..112: note = "Description of Artificial Sequence: Synthetic polypeptide"
DVVMTQSPDSLAVSLGERATINCRSSQSLVHSNRYTYLHWYQQKPGQSPKLLIYKVSNRFSGVPDRFSGSGSGTDFTLKISRVEAEDVGVYYCSQSTRVPYTFGQGTKLEIK

SEQ ID NO: 78 - protein, 123 aa, synthetic construct
  REGION 1..123: note = "Description of Artificial Sequence: Synthetic polypeptide"
QVQLVQSGAEVKKPGASVKVSCKASGYAFSSQWMNWVRQAPGQRLEWIGRIYPGGGDTNYAGKFQGRVTITADTSASTAYMELSSLRSEDTAVYYCARLLRNQPGESYAMDYWGQGTLVTVSS

SEQ ID NO: 79 - protein, 118 aa, synthetic construct
  REGION 1..118: note = "Description of Artificial Sequence: Synthetic polypeptide"
EVQLVESGGGLVQPGGSLRLSCAGSGFTFTDFYMSWVRQAPGKGPEWLSVIRNKANGYTAGYNPSVKGRFTISRDNSKNTLYLQMNSLRAEDTAVYYCARLTYGFDYWGQGTLVTVSS

SEQ ID NO: 80 - protein, 4 aa, synthetic construct
  REGION 1..4: note = "Description of Artificial Sequence: Synthetic peptide"
NGDS

SEQ ID NO: 81 - protein, 4 aa, synthetic construct
  REGION 1..4: note = "Description of Artificial Sequence: Synthetic peptide"
QGDS

SEQ ID NO: 82 - protein, 4 aa, synthetic construct
  REGION 1..4: note = "Description of Artificial Sequence: Synthetic peptide"
NGES

SEQ ID NO: 83 - protein, 4 aa, synthetic construct
  REGION 1..4: note = "Description of Artificial Sequence: Synthetic peptide"
QGES